(19) United States
(12) Patent Application Publication
(10) Pub. No.: US 2024/0410900 A1
(43) Pub. Date: Dec. 12, 2024

(54) MULTIVALENT ANTI-VARIANT FC-REGION ANTIBODIES AND METHODS OF USE

(71) Applicant: Hoffmann-La Roche Inc., Little Falls, NJ (US)

(72) Inventors: Christian KUENZEL, Penzberg (DE); Achim LUTZ; Uwe WESSELS, Penzberg (DE)

(73) Assignee: Hoffmann-La Roche Inc., Little Falls, NJ (US)

(21) Appl. No.: 18/442,986

(22) Filed: Feb. 15, 2024

Related U.S. Application Data
(63) Continuation of application No. PCT/EP2022/072894, filed on Aug. 17, 2022.

(30) Foreign Application Priority Data
Aug. 19, 2021 (EP) 21192040.0

(51) Int. Cl. G01N 33/68; C07K 16/42; G01N 33/535; G01N 33/543; G01N 33/96

(52) U.S. Cl. CPC G01N 33/6854; C07K 16/42; G01N 33/535; G01N 33/54386; G01N 33/96; C07K 2317/24; C07K 2317/35; C07K 2319/61; G01N 2470/04

(57) ABSTRACT

The current invention is directed to an antibody comprising at least four binding sites specifically binding to an immunoglobulin Fc-region of the human IgG1 subclass comprising one, two, three or four amino acid changes compared to a wild-type Fc-region of the human IgG1 subclass for use as positive control and calibration standard in an immunoassay for the detection and quantification of anti-drug antibodies against said one, two, three or four amino acid changes in the Fc-region of the drug antibody.

### CROSS-REFERENCE TO RELATED APPLICATIONS

This application is a continuation of International Application No. PCT/EP2022/072894 having an international filing date of Aug. 17, 2022, which claims benefit of priority to European Patent Application No. 21192040.0, filed Aug. 19, 2021, each of which is incorporated herein by reference in its entirety.

### SEQUENCE LISTING

This application contains a Sequence Listing which has been submitted electronically in XML format and is hereby incorporated by reference in its entirety. Said XML copy, created on May 31, 2024, is named P37030-US_SL.xml and is 42,640 bytes in size.

### FIELD OF THE INVENTION

The present invention relates to multivalent antibodies, especially valency-enhanced and multimers of antibodies specifically binding to variant Fc-regions (multivalent anti-variant Fc-region antibodies), which can be used as positive control as well as calibration standard in bridging immunoassays and in domain-detection assays. The multivalent antibodies according to the current invention specifically bind to variant Fc-regions while not binding to the corresponding wild-type Fc-region and, thus, can specifically bridge two of said variant Fc-regions in an immunoassay, e.g. as positive control or calibration standard. Also reported herein are methods for their production and uses thereof.

### BACKGROUND

Since the development of the first monoclonal antibodies by Koehler and Milstein in 1974, many efforts have been dedicated to the development of antibodies that are appropriate for therapy in humans. The first monoclonal antibodies that became available had been developed in mice and rats. These antibodies when used for therapy of a human being caused unwanted side effects due to the induced immune response, i.e. the formation of anti-rodent antibodies. Many efforts have been dedicated to the reduction or even elimination of such unwanted side effects.

A quite significant number of human or humanized monoclonal antibodies is currently under investigation and needs to be studied in experimental animals, before entry into humans can be considered for the first trial purposes.

Beside standard antibodies having a human wild-type Fc-region more and more antibodies with a variant Fc-region are being developed. These variations in general can also induce an immune response as being non-natural.

Important criteria like bio-availability and immunogenicity just to mention two of them have to be studied by the aid of experimental animals. These studies require, amongst other things, the quantification of anti-drug antibodies in the background of the host's own antibodies. In most cases, mammals are used as experimental animals. Toxicology often is first assessed in rodents like mice or rats. In the more advanced stages of drug development, especially before entry of the drug into human beings, even monkeys have to be included into such pre-clinical studies.

Presently, the enzyme linked immunosorbent sandwich assay (ELISA) in a bridging format represents the state of the art assay format for immunogenicity testing due to its high throughput and sensitivity and its easy applicability to different projects (Mikulskis, A., et al., J. Immunol. Meth. 365 (2011) 38-49).

Standard solid-phase anti-drug antibody immunoassays with monoclonal antibodies involve the formation of a complex between the drug antibody adsorbed on or bound to a solid phase (capture antibody), the anti-drug antibody, and the drug antibody conjugated to a detectable label, e.g. an enzyme (tracer antibody). Thus, a sandwich is formed: solid phase-capture antibody-anti-drug antibody-tracer antibody. In the reaction catalyzed by the sandwich, the activity of the antibody-conjugated enzyme is proportional to the anti-drug antibody concentration. The standard sandwich method is also called bridging immunoassay because the anti-drug antibody bridges between the capture and tracer antibodies, i.e. the drug antibody. Immunoassays such as the bridging ELISA are common assay types in the investigation of an immunogenic answer of a patient to an antibody drug.

Mire-Sluis, A. R., et al., J. Immunol. Methods 289 (2004) 1-16, summarized the recommendations for the design and optimization of immunoassays using detection of host antibodies against biotechnology products.

Wadhwa, M., et al., J. Immunol. Methods 278 (2003) 1-17, reported strategies for the detection, measurement and characterization of unwanted antibodies induced by therapeutic biologicals.

The principles of different immunoassays are described, for example, by Hage, D. S., Anal. Chem. 71 (1999) 294R-304R.

Lu, B., et al., Analyst. 121 (1996) 29R-32R, reported the orientated immobilization of antibodies for the use in immunoassays.

Avidin-biotin-mediated immunoassays are reported, for example, by Wilchek, M., and Bayer, E. A., Methods Enzymol. 184 (1990) 467-469.

In WO 2017/072210 anti-variant Fc-region antibodies and methods of use thereof have been reported.

Wessels, U., et al., Bioanal. 9 (2017) 849-859, reported novel drug and soluble target tolerant antidrug antibody assay for therapeutic antibodies bearing the P329G mutation.

In US 2008/0118939 a conjugate and its use as a standard in an immunoassay has been reported.

### SUMMARY OF THE INVENTION

Reliability of bridging anti-drug antibody (ADA) assays depends on the availability of at least one functional positive control resulting in a sufficient assay signal above background as well as calibration standards in case the ADA assay is to be used to quantify the ADA. In particular, the detection of ADAs to a modification in the Fc-region, especially if the modification is present in both chains of the Fc-region of the drug antibody (therapeutic antibody), is not trivial. In addition, domain-detection-assays also require a positive control with suitable sensitivity.

Antibodies specifically binding to a variant Fc-region and not substantially binding to a wild-type Fc-region are termed anti-variant Fc-region antibodies.

Without being bound by this theory, a standard, bivalent Y-shaped anti-variant Fc-region antibody can bind with both its binding specificities simultaneously to a single Fc-region. Thereby both binding sites are blocked and the formation of a bridging complex is no longer possible. Likewise, the anti-variant Fc-region antibody can bind in a sterically non-favored orientation so that bridging, i.e. simultaneous binding to the capture and tracer drug antibody, is prevented. Thus, there is the need for functional positive controls as well as calibration standards in anti-drug antibody bridging ELISAs.

The current invention is based at least in part on the finding that a monomer of a standard Y-shaped bivalent antibody specifically binding to an immunoglobulin Fc-region of the human IgG1 subclass comprising one, two, three or four amino acid changes (variant human IgG1 Fc-region) compared to a wild-type Fc-region of the human IgG1 subclass cannot bind to such an extent to two variant Fc-region simultaneously, i.e. from a bridge between two variant Fc-regions, that it is suitable as positive control or calibration standard in a bridging ADA assay. It has been found that by increasing the valency above two in an antibody specifically binding to an immunoglobulin Fc-region of the human IgG1 subclass comprising one, two, three or four amino acid changes compared to a wild-type Fc-region of the human IgG1 subclass, e.g. by the addition of further binding sites or by the formation of multimers, in contrast to a bivalent antibody can bind to two variant Fc-regions simultaneously, i.e. can form a bridge and link two variant Fc-regions.

The current invention provides for drug antibodies lacking, e.g. ADCC, Fc-region effector functions, e.g. by introduction of a Pro329Gly (PG) substitution within the Fc-region, a functional positive control as well as calibration standard for use in ADA assays. The functional positive control and calibration standard according to the current invention is either a valency-enhanced or a multimer of an antibody specific for the substitution within the Fc-region of a drug antibody, e.g. a tetravalent or multimeric anti-PG antibody.

The multivalent antibody according to the current invention in combination with a bridging ADA assay now allows for a detailed ADA characterization of clinical samples, as on the one hand the proper function of the assay can be determined and on the other hand the calibration of the assay is possible. With the multivalent antibody according to the invention ADA immunoassays in bridging format are complemented for in-depth characterization of individual ADA-responses against Fc-region-modified drug antibodies.

One aspect according to the invention is an antibody comprising at least three binding sites specifically binding to an immunoglobulin Fc-region of the human IgG1 subclass comprising one, two, three or four amino acid changes compared to a wild-type Fc-region of the human IgG1 subclass.

One aspect according to the invention is a multimer of an antibody specifically binding to an immunoglobulin Fc-region of the human IgG1 subclass comprising one, two, three or four amino acid changes compared to a wild-type Fc-region of the human IgG1 subclass.

One aspect according to the invention is an antibody comprising at least three binding sites specifically binding to an immunoglobulin Fc-region of the human IgG1 subclass comprising at position 329 the amino acid residue glycine (numbering according to Kabat EU index) or a multimer of a (divalent) (Fab′)₂ fragment of an antibody specifically binding to an immunoglobulin Fc-region of the human IgG1 subclass comprising at position 329 the amino acid residue glycine (numbering according to Kabat EU index), wherein the binding sites comprise
(1)
   (a) a HVR-H1 comprising the amino acid sequence of SEQ ID NO: 09;
   (b) a HVR-H2 comprising the amino acid sequence of SEQ ID NO: 12;
   (c) a HVR-H3 comprising the amino acid sequence of SEQ ID NO: 16;
   (d) a HVR-L1 comprising the amino acid sequence of SEQ ID NO: 23;
   (e) a HVR-L2 comprising the amino acid sequence of SEQ ID NO: 26; and
   (f) a HVR-L3 comprising the amino acid sequence of SEQ ID NO: 28; or
(2)
   (a) a HVR-H1 comprising the amino acid sequence of SEQ ID NO: 10;
   (b) a HVR-H2 comprising the amino acid sequence of SEQ ID NO: 13;
   (c) a HVR-H3 comprising the amino acid sequence of SEQ ID NO: 17;
   (d) a HVR-L1 comprising the amino acid sequence of SEQ ID NO: 23 or 24;
   (e) a HVR-L2 comprising the amino acid sequence of SEQ ID NO: 26; and
   (f) a HVR-L3 comprising the amino acid sequence of SEQ ID NO: 29; or
(3)
   (a) a HVR-H1 comprising the amino acid sequence of SEQ ID NO: 10;
   (b) a HVR-H2 comprising the amino acid sequence of SEQ ID NO: 14;
   (c) a HVR-H3 comprising the amino acid sequence of SEQ ID NO: 18;
   (d) a HVR-L1 comprising the amino acid sequence of SEQ ID NO: 23 or 24;
   (e) a HVR-L2 comprising the amino acid sequence of SEQ ID NO: 26; and
   (f) a HVR-L3 comprising the amino acid sequence of SEQ ID NO: 30; or
(4)
   a combination of any one of (1) to (3);
with the HVRs determined according to Kabat.

In certain embodiments of all aspects and embodiments of the invention, the at least trivalent antibody (antibody comprising at least three binding sites) is a trivalent, a tetravalent, a hexavalent, an octavalent, or a decavalent antibody. In one preferred embodiment, the at least trivalent antibody is a tetravalent antibody.

In certain embodiments of all aspects and embodiments of the invention, the at least trivalent antibody (antibody comprising at least three binding sites) is an IgA or an IgM antibody.

One aspect according to the invention is a multimer of an antibody specifically binding to an immunoglobulin Fc-region of the human IgG1 subclass comprising at position 329 the amino acid residue glycine (numbering according to Kabat EU index) or a multimer of a (divalent) (Fab′)₂ fragment of an antibody specifically binding to an immunoglobulin Fc-region of the human IgG1 subclass comprising at position 329 the amino acid residue glycine (numbering according to Kabat EU index), wherein the antibody or the (Fab′)₂ fragment comprises
(1)
   (a) a HVR-H1 comprising the amino acid sequence of SEQ ID NO: 09;
   (b) a HVR-H2 comprising the amino acid sequence of SEQ ID NO: 12;
   (c) a HVR-H3 comprising the amino acid sequence of SEQ ID NO: 16;
   (d) a HVR-L1 comprising the amino acid sequence of SEQ ID NO: 23;
   (e) a HVR-L2 comprising the amino acid sequence of SEQ ID NO: 26; and
   (f) a HVR-L3 comprising the amino acid sequence of SEQ ID NO: 28; or
(2)
   (a) a HVR-H1 comprising the amino acid sequence of SEQ ID NO: 10;
   (b) a HVR-H2 comprising the amino acid sequence of SEQ ID NO: 13;
   (c) a HVR-H3 comprising the amino acid sequence of SEQ ID NO: 17;
   (d) a HVR-L1 comprising the amino acid sequence of SEQ ID NO: 23 or 24;
   (e) a HVR-L2 comprising the amino acid sequence of SEQ ID NO: 26; and
   (f) a HVR-L3 comprising the amino acid sequence of SEQ ID NO: 29; or
(3)
   (a) a HVR-H1 comprising the amino acid sequence of SEQ ID NO: 10;
   (b) a HVR-H2 comprising the amino acid sequence of SEQ ID NO: 14;
   (c) a HVR-H3 comprising the amino acid sequence of SEQ ID NO: 18;
   (d) a HVR-L1 comprising the amino acid sequence of SEQ ID NO: 23 or 24;
   (e) a HVR-L2 comprising the amino acid sequence of SEQ ID NO: 26; and
   (f) a HVR-L3 comprising the amino acid sequence of SEQ ID NO: 30;
   with the HVRs determined according to Kabat.

In one preferred embodiment of all aspects and embodiments of the invention, the antibody or (Fab′)₂ fragment specifically binds to an immunoglobulin Fc-region of the human IgG1 subclass comprising at position 329 the amino acid residue glycine and at positions 234 and 235 the amino acid residue alanine (numbering according to Kabat EU index).

In certain embodiments of all aspects and embodiments of the invention, the multimer is a dimer, a trimer, a tetramer, a pentamer, a hexamer, a heptamer, an octamer, a nonamer, or a decamer.

One aspect according to the invention is the use of an at least trivalent antibody according to the invention as positive control in an in vitro (bridging) immunoassay.

One aspect according to the invention is the use of a multimer according to the invention as positive control in an in vitro (bridging) immunoassay.

One aspect according to the current invention is the use of an at least trivalent antibody according to the invention as standard in an in vitro (bridging) immunoassay. In certain embodiments, the use is for the generation of a calibration function. In one preferred embodiment, the calibration function is for quantitative determination of anti-drug antibodies against a drug antibody, wherein the anti-drug antibodies bind to one or more amino acid residue(s) in the Fc-region of the drug antibody that is altered compared to a wild-type Fc-region.

One aspect according to the current invention is the use of a multimer according to the invention as standard in an in vitro (bridging) immunoassay. In certain embodiments, the use is for the generation of a calibration function. In one preferred embodiment, the calibration function is for quantitative determination of anti-drug antibodies against a drug antibody, wherein the anti-drug antibodies bind to one or more amino acid residue(s) in the Fc-region of the drug antibody that is altered compared to a wild-type Fc-region.

In certain embodiments of all aspects and embodiments of the invention, the in vitro (bridging) immunoassay is for the determination of anti-drug antibodies against a drug antibody, wherein the anti-drug antibodies bind to the Fc-region of the drug antibody. In certain embodiments, the anti-drug antibody binds to one or more amino acid residue(s) in the Fc-region of the drug antibody that is altered compared to a wild-type Fc-region.

In certain embodiments of all aspects and embodiments of the invention, the in vitro immunoassay is an in vitro bridging ELISA.

In certain embodiments of all aspects and embodiments of the invention, the drug antibody comprises an immunoglobulin Fc-region of the human IgG1 subclass comprising at position 329 the amino acid residue glycine (numbering according to Kabat EU index).

In one preferred embodiment of all aspects and embodiments of the invention, the drug antibody comprises an immunoglobulin Fc-region of the human IgG1 subclass comprising at position 329 the amino acid residue glycine and at positions 234 and 235 the amino acid residue alanine (numbering according to Kabat EU index).

In one preferred embodiment of all aspects and embodiments of the invention, the in vitro immunoassay is an in vitro bridging immunoassay for the determination of anti-drug antibodies comprising as capture and as tracer antibody the drug antibody.

One aspect according to the invention is an in vitro immunoassay for the determination of the presence and/or amount of anti-drug antibodies in a (serum containing) sample,
wherein the anti-drug antibodies bind to at least one amino acid residue in the Fc-region of the drug antibody that is altered compared to a wild-type Fc-region,
wherein the immunoassay comprises as capture and as tracer antibody the drug antibody, characterized in that
the at least trivalent antibody according to the invention or the multimer according to the invention is used as positive control or as calibration standard in the immunoassay.

In certain embodiments of all aspects and embodiments of the invention, the use as calibration standard is for the generation of a calibration function. In one preferred embodiment, the calibration function is for quantitative determination of anti-drug antibodies against a drug antibody, wherein the anti-drug antibodies bind to at least one amino acid residue in the Fc-region of the drug antibody that is altered compared to a wild-type Fc-region

In certain embodiments of all aspects and embodiments of the invention, the antibody specifically binding to the variant immunoglobulin Fc-region of the human IgG1 subclass is a monoclonal antibody.

In certain embodiments of all aspects and embodiments of the invention, the drug antibody is a human, humanized, or chimeric antibody.

One aspect is a method of producing a multimer according to the invention comprising chemically cross-linking a full-length antibody specifically binding to an immunoglobulin Fc-region of the human IgG1 subclass comprising at position 329 the amino acid residue glycine (numbering according to Kabat EU index), wherein the antibody comprises
(1)
   (a) a HVR-H1 comprising the amino acid sequence of SEQ ID NO: 09;
   (b) a HVR-H2 comprising the amino acid sequence of SEQ ID NO: 12;
   (c) a HVR-H3 comprising the amino acid sequence of SEQ ID NO: 16;
   (d) a HVR-L1 comprising the amino acid sequence of SEQ ID NO: 23;
   (e) a HVR-L2 comprising the amino acid sequence of SEQ ID NO: 26; and
   (f) a HVR-L3 comprising the amino acid sequence of SEQ ID NO: 28; or
(2)
   (a) a HVR-H1 comprising the amino acid sequence of SEQ ID NO: 10;
   (b) a HVR-H2 comprising the amino acid sequence of SEQ ID NO: 13;
   (c) a HVR-H3 comprising the amino acid sequence of SEQ ID NO: 17;
   (d) a HVR-L1 comprising the amino acid sequence of SEQ ID NO: 23 or 24;
   (e) a HVR-L2 comprising the amino acid sequence of SEQ ID NO: 26; and
   (f) a HVR-L3 comprising the amino acid sequence of SEQ ID NO: 29; or
(3)
   (a) a HVR-H1 comprising the amino acid sequence of SEQ ID NO: 10;
   (b) a HVR-H2 comprising the amino acid sequence of SEQ ID NO: 14;
   (c) a HVR-H3 comprising the amino acid sequence of SEQ ID NO: 18;
   (d) a HVR-L1 comprising the amino acid sequence of SEQ ID NO: 23 or 24;
   (e) a HVR-L2 comprising the amino acid sequence of SEQ ID NO: 26; and
   (f) a HVR-L3 comprising the amino acid sequence of SEQ ID NO: 30;
   with the HVRs determined according to Kabat
   using N-succinimidyl-3-acetylthiopropionate (SATP) and maleimidohexanoyl-N-hydroxysuccinimide (MHS).

### BRIEF DESCRIPTION OF THE FIGURES

FIG. 1 Scheme of an immunoassay using the multimer according to the invention as positive control or calibration standard.

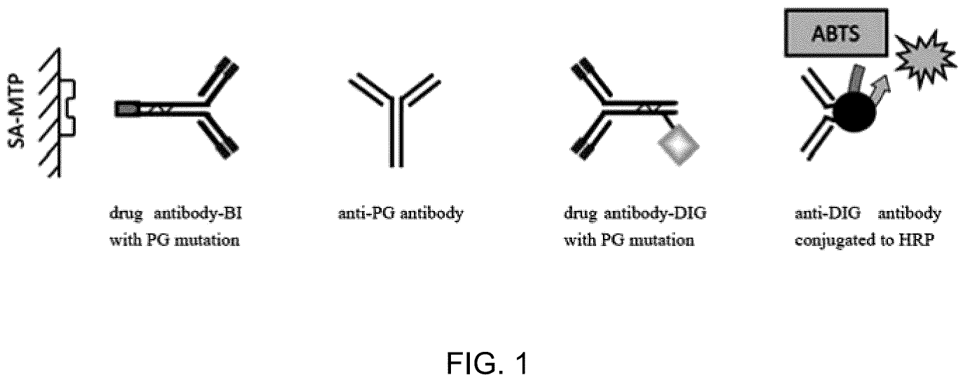

FIG. 2 Signal obtained with monomeric anti-PG antibody clone 1.7.24 in a bridging immunoassay, with the same but differently derivatized drug antibodies as capture and tracer antibody

FIG. 3 Different modes of binding of anti-variant Fc-region antibody to an Fc-region; (A): binding to a single Fc-region with both binding sites at the same time; (B) steric hindrance.

FIG. 4 Modes of multimerizing an anti-variant Fc-region antibody; (A) recombinant expression; (B) chemical cross-linking.

FIG. 5 SEC-chromatogram of pools of cross-linked anti-PG antibody with different degrees of cross-linking, i.e. molecule size.

FIG. 6 Signal obtained with multimeric anti-PG antibody clone 1.7.24 in a bridging immunoassay, with the same but differently derivatized drug antibodies as capture and tracer antibody.

FIG. 7 Signal of monomeric anti-PG antibody and multimeric anti-PG antibody according to the current invention shown as direct 1:1 comparison in a bridging immunoassay, with the same but differently derivatized drug antibodies as capture and tracer antibody.

FIG. 8 Signal-to-noise ratio of monomeric anti-PG antibody and multimeric anti-PG antibody according to the current invention shown as direct 1:1 comparison in a bridging immunoassay, with the same but differently derivatized drug antibodies as capture and tracer antibody.

FIG. 9 Multimeric anti-PG antibody according to the current invention in a bridging immunoassay with different formats of drug antibodies used as capture and tracer antibody (both same format).

FIG. 10 Signal of tetravalent anti-PG antibody used as calibration standard in a bridging immunoassay, with the same but differently derivatized drug antibodies Fc-regions as capture and tracer antibody.

FIG. 11 Signal of the anti-PG antibody in IgM format used as calibration standard in a bridging immunoassay, with the variant Fc-region as capture agent and the drug antibody in TCB format as tracer antibody.

### DETAILED DESCRIPTION OF EMBODIMENTS OF THE INVENTION

### I. Definitions

As used herein, the amino acid positions of all constant regions and domains of the heavy and light chain are numbered according to the Kabat numbering system described in Kabat, et al., Sequences of Proteins of Immunological Interest, 5th ed., Public Health Service, National Institutes of Health, Bethesda, MD (1991) and is referred to as “numbering according to Kabat” herein. Specifically the Kabat numbering system (see pages 647-660) of Kabat, et al., Sequences of Proteins of Immunological Interest, 5th ed., Public Health Service, National Institutes of Health, Bethesda, MD (1991) is used for the light chain constant domain CL of kappa and lambda isotype and the Kabat EU index numbering system (see pages 661-723) is used for the constant heavy chain domains (CH1, Hinge, CH2 and CH3).

“Affinity” refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity that reflects a 1:1 interaction between members of a binding pair (e.g., antibody and antigen). The affinity of a molecule X for its partner Y can generally be represented by the dissociation constant (k_{d}). Affinity can be measured by common methods known in the art, including those described herein.

The term “(amino acid) alteration” denotes the replacement of at least one amino acid residue in a predetermined parent amino acid sequence with a different “replacement” amino acid residue to generate a variant amino acid sequence. The replacement residue or residues may be a “naturally occurring amino acid residue” (i.e. encoded by the genetic code) and selected from the group consisting of: alanine (Ala); arginine (Arg); asparagine (Asn); aspartic acid (Asp); cysteine (Cys); glutamine (Gln); glutamic acid (Glu); glycine (Gly); histidine (His); isoleucine (Ile); leucine (Leu); lysine (Lys); methionine (Met); phenylalanine (Phe); proline (Pro); serine (Ser); threonine (Thr); tryptophan (Trp); tyrosine (Tyr); and valine (Val). In certain embodiments, the replacement residue is not cysteine. Substitution with non-naturally occurring amino acid residues is also encompassed by the definition of an amino acid alteration herein. A “non-naturally occurring amino acid residue” denotes a residue, other than those naturally occurring amino acid residues listed above, which is able to covalently bind adjacent amino acid residues(s) in a polypeptide chain. Examples of non-naturally occurring amino acid residues include norleucine, ornithine, norvaline, homoserine, aib and other amino acid residue analogues such as those described in Ellman, et al., Meth. Enzym. 202 (1991) 301-336. To generate such non-naturally occurring amino acid residues, the procedures of Noren, et al. (Science 244 (1989) 182) and/or Ellman, et al. (supra) can be used. Briefly, these procedures involve chemically activating a suppressor tRNA with a non-naturally occurring amino acid residue followed by in vitro transcription and translation of the RNA. Non-naturally occurring amino acids can also be incorporated into peptides via chemical peptide synthesis and subsequent fusion of these peptides with recombinantly produced polypeptides, such as antibodies or antibody fragments.

Within this application, whenever an amino acid alteration is mentioned it is a deliberated amino acid alteration and not a random amino acid modification.

The terms “anti-variant (human) Fc-region antibody” and “an antibody that specifically binds to a variant (human) Fc-region” refer to an antibody that is capable of binding a variant (human) Fc-region with sufficient affinity such that the antibody is useful as a diagnostic agent in targeting a variant (human) Fc-region. In certain embodiments, the extent of binding of an anti-variant (human) Fc-region antibody to the corresponding wild-type (human) Fc-region is less than about 10% of the binding of the antibody to the variant (human) Fc-region. This can be determined e.g. using Surface Plasmon Resonance. In certain embodiments, an antibody that specifically binds to a variant (human) Fc-region has a dissociation constant (K_{D}) of 10⁻⁸ M or less, e.g. from 10⁻⁸ M to 10⁻¹² M).

The term “drug antibody” herein is used in the broadest sense and encompasses various antibody structures, including but not limited to monoclonal antibodies, polyclonal antibodies, as well as multispecific antibodies (e.g., bispecific antibodies), so long as they exhibit the desired antigen-binding activity.

The term “binding to” denotes the binding of a first entity to a second entity, such as e.g. of an antibody to its antigen. This binding can be determined using, for example, a BIAcore® assay (GE Healthcare, Uppsala, Sweden).

For example, in one possible embodiment of the BIAcore® assay, the antigen is bound to a surface and binding of the antibody is measured by surface plasmon resonance (SPR).

The affinity of the binding is defined by the terms kₐ (association constant: rate constant for the association to form a complex), k_{d} (dissociation constant; rate constant for the dissociation of the complex), and K_{D} (k_{d}/kₐ). Alternatively, the binding signal of a SPR sensorgram can be compared directly to the response signal of a reference, with respect to the resonance signal height and the dissociation behaviors.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

“Effector functions” refer to those biological activities attributable to the Fc-region of an antibody, which vary with the antibody class. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

Fc receptor binding dependent effector functions can be mediated by the interaction of the Fc-region of an antibody with Fc receptors (FcRs), which are specialized cell surface receptors on hematopoietic cells. Fc receptors belong to the immunoglobulin superfamily, and have been shown to mediate both the removal of antibody-coated pathogens by phagocytosis of immune complexes, and the lysis of erythrocytes and various other cellular targets (e.g. tumor cells) coated with the corresponding antibody, via antibody dependent cell mediated cytotoxicity (ADCC) (see e.g. Van de Winkel, J. G. and Anderson, C. L., J. Leukoc. Biol. 49 (1991) 511-524). FcRs are defined by their specificity for immunoglobulin isotypes: Fc receptors for IgG antibodies are referred to as FcγR. Fc receptor binding is described e.g. in Ravetch, J. V. and Kinet, J. P., Annu. Rev. Immunol. 9 (1991) 457-492; Capel, P. J., et al., Immunomethods 4 (1994) 25-34; de Haas, M., et al., J. Lab. Clin. Med. 126 (1995) 330-341; Gessner, J. E., et al., Ann. Hematol. 76 (1998) 231-248.

Cross-linking of receptors for the Fc-region of IgG antibodies (FcγR) triggers a wide variety of effector functions including phagocytosis, antibody-dependent cellular cytotoxicity, and release of inflammatory mediators, as well as immune complex clearance and regulation of antibody production. In humans, three classes of FcγR have been characterized, which are:
FcγRI (CD64) binds monomeric IgG with high affinity and is expressed on macrophages, monocytes, neutrophils and eosinophils. Modification in the Fc-region IgG at least at one of the amino acid residues E233-G236, P238, D265, N297, A327 and P329 (numbering according to EU index of Kabat) reduce binding to FcγRI. IgG2 residues at positions 233-236, substituted into IgG1 and IgG4, reduced binding to FcγRI by 103-fold and eliminated the human monocyte response to antibody-sensitized red blood cells (Armour, K. L., et al., Eur. J. Immunol. 29 (1999) 2613-2624).
FcγRII (CD32) binds complexed IgG with medium to low affinity and is widely expressed. This receptor can be divided into two sub-types, FcγRIIA and FcγRIIB. FcγRIIA is found on many cells involved in killing (e.g. macrophages, monocytes, neutrophils) and seems able to activate the killing process. FcγRIIB seems to play a role in inhibitory processes and is found on B-cells, macrophages and on mast cells and eosinophils. On B-cells it seems to function to suppress further immunoglobulin production and isotype switching to, for example, the IgE class. On macrophages, FcγRIIB acts to inhibit phagocytosis as mediated through FcγRIIA. On eosinophils and mast cells the B-form may help to suppress activation of these cells through IgE binding to its separate receptor. Reduced binding for FcγRIIA is found e.g. for antibodies comprising an IgG Fc-region with mutations at least at one of the amino acid residues E233-G236, P238, D265, N297, A327, P329, D270, Q295, A327, R292, and K414 (numbering according to EU index of Kabat).
FcγRIII (CD16) binds IgG with medium to low affinity and exists as two types. FcγRIIIA is found on NK cells, macrophages, eosinophils and some monocytes and T cells and mediates ADCC. FcγRIIIB is highly expressed on neutrophils. Reduced binding to FcγRIIIA is found e.g. for antibodies comprising an IgG Fc-region with mutation at least at one of the amino acid residues E233-G236, P238, D265, N297, A327, P329, D270, Q295, A327, S239, E269, E293, Y296, V303, A327, K338 and D376 (numbering according to EU index of Kabat).

Mapping of the binding sites on human IgG1 for Fc receptors, the above mentioned mutation sites and methods for measuring binding to FcγRI and FcγRIIA are described in Shields, R. L., et al. J. Biol. Chem. 276 (2001) 6591-6604.

The term “Fc receptor” as used herein refers to activation receptors characterized by the presence of a cytoplasmic ITAM sequence associated with the receptor (see e.g. Ravetch, J. V. and Bolland, S., Annu. Rev. Immunol. 19 (2001) 275-290). Such receptors are FcγRI, FcγRIIA and FcγRIIIA. The term “no binding of FcγR” denotes that at an antibody concentration of 10 μg/mL the binding of an antibody as reported herein to NK cells is 10% or less of the binding found for anti-OX40L antibody LC.001 as reported in WO 2006/029879.

While IgG4 shows reduced FcR binding, antibodies of other IgG subclasses show strong binding. However Pro238, Asp265, Asp270, Asn297 (loss of Fc carbohydrate), Pro329 and 234, 235, 236 and 237 Ile253, Ser254, Lys288, Thr307, Gln311, Asn434, and His435 are residues which provide if altered also reduce FcR binding (Shields, R. L., et al. J. Biol. Chem. 276 (2001) 6591-6604; Lund, J., et al., FASEB J. 9 (1995) 115-119; Morgan, A., et al., Immunology 86 (1995) 319-324; and EP 0 307 434).

The term “Fc-region” herein is used to define a C-terminal region of an immunoglobulin heavy chain that contains at least a portion of the constant region. The term includes native sequence Fc-regions and variant Fc-regions. In certain embodiments, a human IgG heavy chain Fc-region extends from Cys226, or from Pro230, to the carboxyl-terminus of the heavy chain. However, the C-terminal lysine (Lys447) of the Fc-region may or may not be present. Unless otherwise specified herein, numbering of amino acid residues in the Fc-region or constant region is according to the EU numbering system, also called the EU index, as described in Kabat, E. A. et al., Sequences of Proteins of Immunological Interest, 5th ed., Public Health Service, National Institutes of Health, Bethesda, MD (1991), NIH Publication 91-3242.

The Fc-region of an antibody is directly involved in complement activation, C1q binding, C3 activation and Fc receptor binding. While the influence of an antibody on the complement system is dependent on certain conditions, binding to C1q is caused by defined binding sites in the Fc-region. Such binding sites are known in the state of the art and described e.g. by Lukas, T. J., et al., J. Immunol. 127 (1981) 2555-2560; Brunhouse, R., and Cebra, J. J., Mol. Immunol. 16 (1979) 907-917; Burton, D. R., et al., Nature 288 (1980) 338-344; Thommesen, J. E., et al., Mol. Immunol. 37 (2000) 995-1004; Idusogie, E. E., et al., J. Immunol. 164 (2000) 4178-4184; Hezareh, M., et al., J. Virol. 75 (2001) 12161-12168; Morgan, A., et al., Immunology 86 (1995) 319-324; and EP 0 307 434. Such binding sites are e.g. L234, L235, D270, N297, E318, K320, K322, P331 and P329 (numbering according to EU index of Kabat; Unless otherwise specified herein, numbering of amino acid residues in the Fc-region or constant region is according to the EU numbering system, also called the EU index, as described in Kabat, E. A. et al., Sequences of Proteins of Immunological Interest, 5th ed., Public Health Service, National Institutes of Health, Bethesda, MD (1991), NIH Publication 91-3242). Antibodies of subclass IgG1, IgG2 and IgG3 usually show complement activation, C1q binding and C3 activation, whereas IgG4 do not activate the complement system, do not bind C1q and do not activate C3. An “Fc-region of an antibody” is a term well known to the skilled artisan and defined on the basis of papain cleavage of antibodies. In certain embodiments, the Fc-region is a human Fc-region. In certain embodiments, the Fc-region of the drug antibody is of the human IgG1 subclass comprising the mutations L234A and L235A (numbering according to EU index of Kabat).

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full length antibody”, “intact antibody”, and “whole antibody” are used herein interchangeably to refer to an antibody having a structure substantially similar to a native antibody structure or having heavy chains that contain an Fc-region as defined herein.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR”, as used herein, refers to each of the regions of an antibody variable domain comprising the amino acid residue stretches which are hypervariable in sequence (“complementarity determining regions” or “CDRs”) and/or form structurally defined loops (“hypervariable loops”), and/or contain the antigen-contacting residues (“antigen contacts”). Generally, antibodies comprise six HVRs; three in the VH (H1, H2, H3), and three in the VL (L1, L2, L3).

HVRs include
(a) hypervariable loops occurring at amino acid residues 26-32 (L1), 50-52 (L2), 91-96 (L3), 26-32 (H1), 53-55 (H2), and 96-101 (H3) (Chothia, C. and Lesk, A. M., J. Mol. Biol. 196 (1987) 901-917);
(b) CDRs occurring at amino acid residues 24-34 (L1), 50-56 (L2), 89-97 (L3), 31-35b (H1), 50-65 (H2), and 95-102 (H3) (Kabat, E. A. et al., Sequences of Proteins of Immunological Interest, 5th ed. Public Health Service, National Institutes of Health, Bethesda, MD (1991), NIH Publication 91-3242);
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262:732-745 (1996)); and
(d) combinations of (a), (b), and/or (c), including amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al . . . supra.

An “isolated” multimer is one that has been separated from a component of its natural environment. In certain embodiments, the multimer is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman, S. et al., J. Chromatogr. B 848 (2007) 79-87.

The term “monoclonal antibody” as used herein refers to an antibody obtained from a population of substantially homogeneous antibodies, i.e., the individual antibodies comprising the population are identical and/or bind the same epitope, except for a minor fraction of antibodies, e.g., containing naturally occurring mutations or arising during production of a monoclonal antibody preparation. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, monoclonal antibodies may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

“Native antibodies” refer to naturally occurring immunoglobulin molecules with varying structures. For example, native IgG antibodies are heterotetrameric glycoproteins of about 150,000 Daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3), whereby between the first and the second constant domain a hinge region is located. Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

The term “variant (human) Fc-region” denotes an amino acid sequence that differs from that of a “wild-type” (human) Fc-region amino acid sequence by virtue of at least one “amino acid alteration”. In certain embodiments, the variant Fc-region has at least one amino acid alteration compared to a native Fc-region, e.g. from about one to about ten amino acid alterations, and in certain embodiments from about one to about five amino acid alterations with respect to a native Fc-region. In certain embodiments, the (variant) Fc-region has at least about 80% homology with a wild-type Fc-region, and in certain embodiments, the variant Fc-region has least about 90% homology, in one preferred embodiment, the variant Fc-region has at least about 95% homology.

The variant Fc-regions are defined by the amino acid alterations that are contained. Thus, for example, the term P329G denotes a variant Fc-region with the mutation of proline to glycine at amino acid position 329 relative to the parent (wild-type) Fc-region. The identity of the wild-type amino acid may be unspecified, in which case the aforementioned variant is referred to as 329G. The term “alteration” denotes a change to naturally occurring amino acids as well as a change to non-naturally occurring amino acids (see e.g. U.S. Pat. No. 6,586,207, WO 98/48032, WO 03/073238, US 2004/0214988, WO 2005/35727, WO 2005/74524, Chin, J. W., et al., J. Am. Chem. Soc. 124 (2002) 9026-9027; Chin, J. W. and Schultz, P. G., ChemBioChem 11 (2002) 1135-1137; Chin, J. W., et al., PICAS United States of America 99 (2002) 11020-11024; Wang, L. and Schultz, P. G., Chem. (2002) 1-10).

The term “wild-type Fc-region” denotes an amino acid sequence identical to the amino acid sequence of an Fc-region found in nature. Wild-type human Fc-regions include a native human IgG1 Fc-region (non-A and A allotypes), native human IgG2 Fc-region, native human IgG3 Fc-region, and native human IgG4 Fc-region as well as naturally occurring variants thereof.

The term “drug antibody” relates to any antibody preparation that is intended for use in a human being as therapeutic. Preferably, such a drug antibody will be a monoclonal antibody. Further preferred such monoclonal antibody will be obtained from a great ape or be a human monoclonal antibody. Preferably, it will be a human monoclonal antibody. Also preferred such drug monoclonal antibody will be a humanized monoclonal antibody.

The term “valent” as used within the current application denotes the presence of a specified number of binding sites in a (antibody) molecule. As such, the terms “bivalent”, “tetravalent”, and “hexavalent” denote the presence of two binding sites, four binding sites, and six binding sites, respectively, in a (antibody) molecule. The at least trivalent antibody according to the invention is in one preferred embodiment “tetravalent”. A “binding site” is formed by a cognate pair of an antibody light chain variable domain (VL) and an antibody heavy chain variable domain (VH).

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt, T. J. et al. Kuby Immunology, 6th ed., W.H. Freeman and Co., N.Y. (2007), page 91) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively (see, e.g., Portolano, S., et al., J. Immunol. 150 (1993) 880-887; Clackson, T., et al., Nature 352 (1991) 624-628).

### II. Compositions and Methods

The invention is based, at least in part, on the finding that a monomeric, bivalent antibody specifically binding to an immunoglobulin Fc-region of the human IgG1 subclass comprising one, two, three or four amino acid changes compared to a wild-type Fc-region of the human IgG1 subclass cannot bind to two variant Fc-region in sufficient sensitivity/amount, i.e. to from a (detectable) bridge between two variant Fc-regions, and cannot be used as positive control or calibration standard in an ADA assay.

It has been found that a multivalent format of an antibody specifically binding to an immunoglobulin Fc-region of the human IgG1 subclass comprising one, two, three or four amino acid changes compared to a wild-type Fc-region of the human IgG1 subclass in contrast to a bivalent form can bind to two variant Fc-regions in sufficient sensitivity/amount, i.e. can link two variant Fc-regions in sufficient amount/sensitivity, so that it is suitable as positive control and/or calibration standard in an ADA assay.

For drug antibodies lacking binding to Fc receptors and/or Fc effector functions, e.g. by introduction of a Pro329Gly (PG) substitution within the Fc-region, a functional positive control as well as a calibration standard for use in ADA assays is reported herein. The functional positive control and the calibration standard according to the current invention is a multivalent form of an antibody specific for an amino acid alteration within the Fc-region of a drug (therapeutic) antibody, e.g. an at least trivalent form or a multimeric form of an anti-PG antibody.

The multivalent antibody according to the current invention in combination with a bridging assay now allows for a detailed ADA characterization of clinical samples, as the proper function of the assay can be determined. With the multivalent antibody according to the invention bridging anti-drug antibody immunoassays are complemented for in-depth characterization of individual ADA-responses against Fc-region-modified drug antibodies.

One aspect according to the invention is a multivalent form of an antibody specifically binding to an immunoglobulin Fc-region of the human IgG1 subclass comprising one, two, three or four amino acid changes compared to a wild-type Fc-region of the human IgG1 subclass.

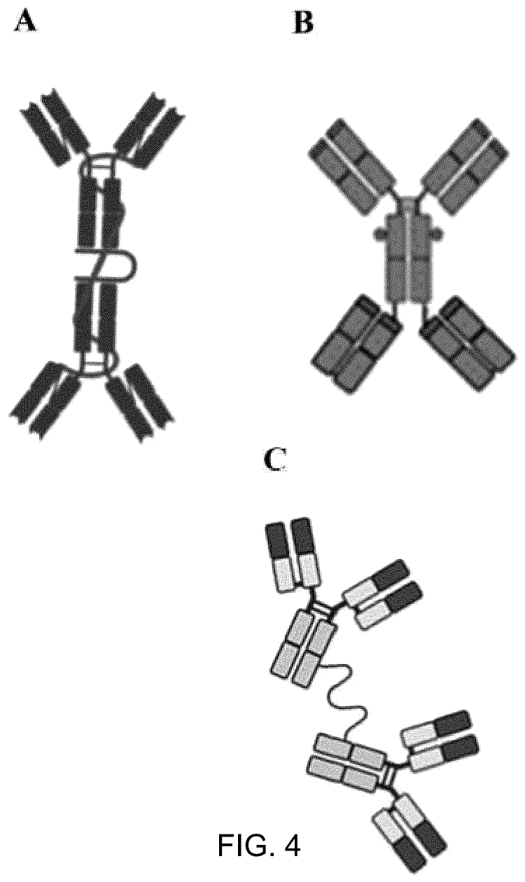

In a first example, the invention is exemplified in the following with a chemically conjugated multimer according to FIG. 4C of an anti-PG antibody, i.e. an antibody specifically binding to a human Fc-region of the IgG subclass with the P329G alteration. This is presented solely to exemplify the invention and shall not be construed as limitation. The true scope is set forth in the appended claims.

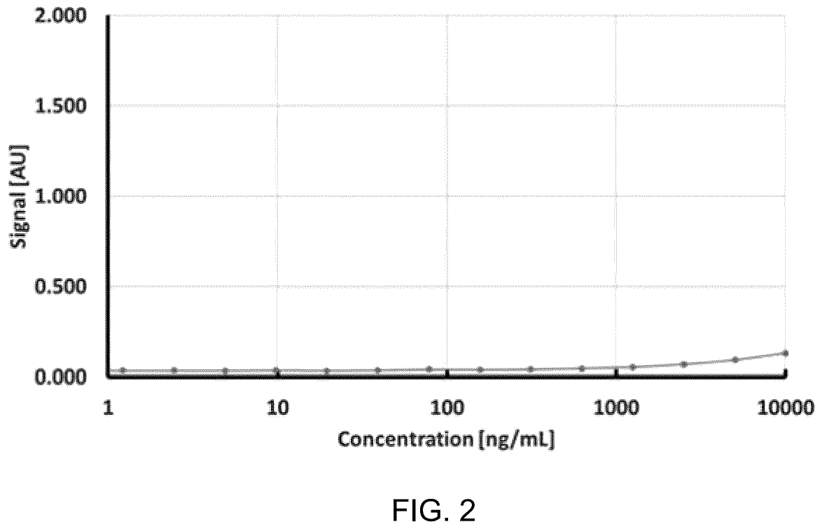

In more detail, monomeric anti-PG antibody clone 1.7.24 was tested for use as positive control in a bridging immunoassay, with the same but differently derivatized drug antibodies as capture and tracer antibody. The results are shown in FIG. 2.

It has been found that up to a concentration of 1,000 ng/mL of the bivalent, Y-shaped (=monomeric) anti-PG antibody clone 1.7.24 in 100% matrix no signal could be detected. Furthermore, at a concentration of 10,000 ng/ml a signal of only 0.13 AU was obtained. The signal-to-noise ratio was >2 AU at a concentration of 2,500 ng/ml of the anti-PG antibody clone 1.7.24 (see Example 4A).

Regulatory authorities require an at least 100 ng/mL threshold. This means the sensitivity of an ADA positive control should reach 100 ng/ml in 100% matrix (see, e.g., a FDA guidance from 2019).

Generally, a signal is deemed to be sufficient for use if it is at least at the double value of a blank sample, i.e. a sample not containing the analyte.

Thus, monomeric anti-PG antibody clone 1.7.24 is not suitable as a positive control or calibration standard in an anti-drug antibody assay due to its low sensitivity.

The same behavior was seen with a different clone, i.e. anti-PG antibody clone 1.3.17. With this monomeric antibody a signal of only 1.85 times the blank value was obtained at 100 ng/ml (see Example 4B). Thus, also this clone is in monomeric form not sensitive enough for use as positive control or calibration standard in an ADA assay.

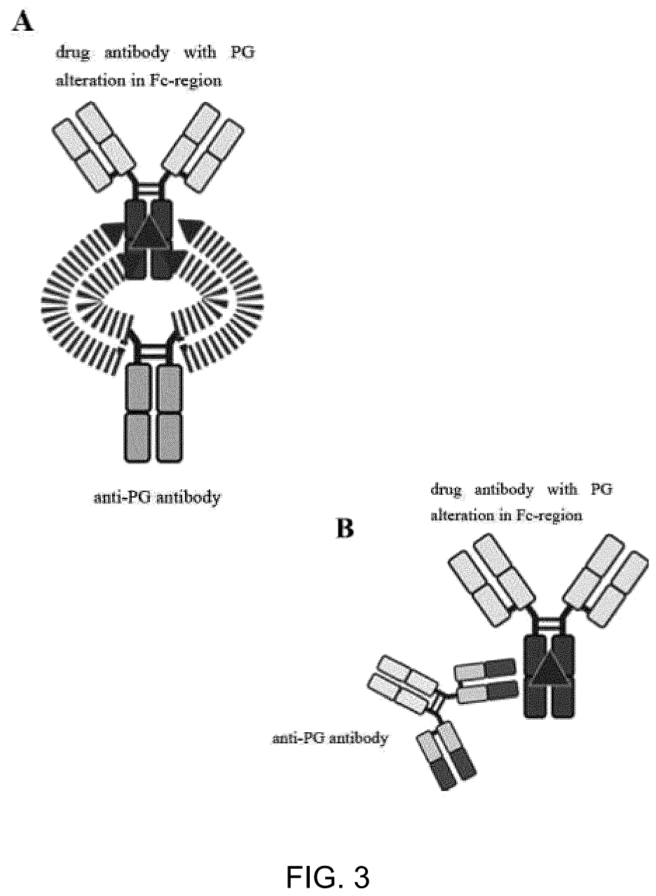

Why can these monomeric anti-PG antibodies not form a bridge in the ADA assay? Without being bound by this theory, it is assumed that either one anti-PG antibody binds one drug antibody molecule with both paratopes (see FIG. 3A) or only one binding site of the anti-PG antibody can bind to the Fc-region while the other becomes sterically blocked (see FIG. 3B).

It has been found that in order to address this problem, the use of a multivalent anti-PG antibody is advantageous.

Any kind of valency enhancement technique can be applied, e.g. recombinant production as dimer or fusion molecule, change to a different antibody format, such as, e.g., IgA (see FIG. 4A) or IgM, addition of binding sites (see FIG. 4B), or chemical conjugation using, e.g. N-succinimidyl-3-acetylthiopropionate (SATP) and maleimidohexanoyl-N-hydroxysuccinimide (MHS) (see FIG. 4C).

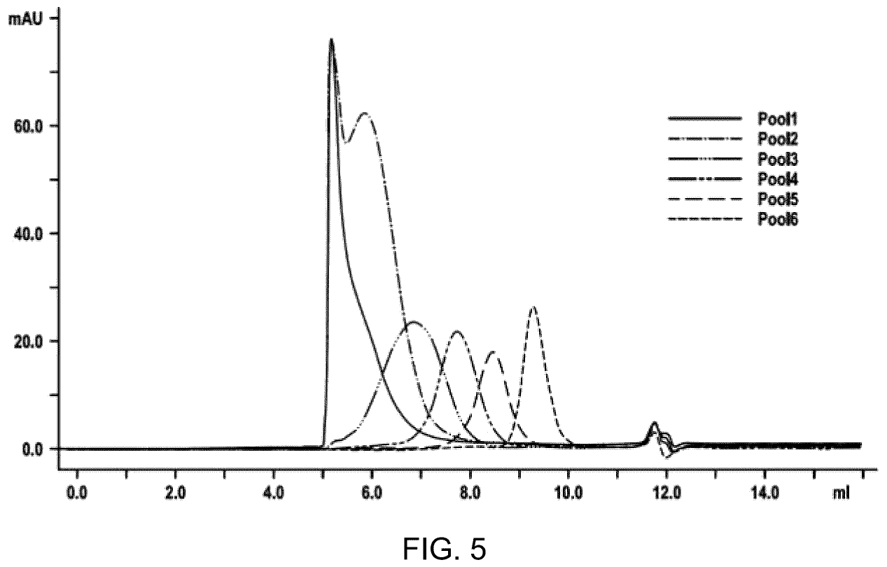

Exemplary chemical conjugation of the anti-PG antibody clone 1.7.24 using N-succinimidyl-3-acetylthiopropionate (SATP) and maleimidohexanoyl-N-hydroxysuccinimide (MHS) has been done. The reaction worked smoothly. The reaction product has been analyzed using SEC and different fractions have been collected. As can be seen from FIG. 5 multimers with different degrees of multimerization can be obtained, i.e. from highly cross-linked multimers (pool 2) to di- and trimers only.

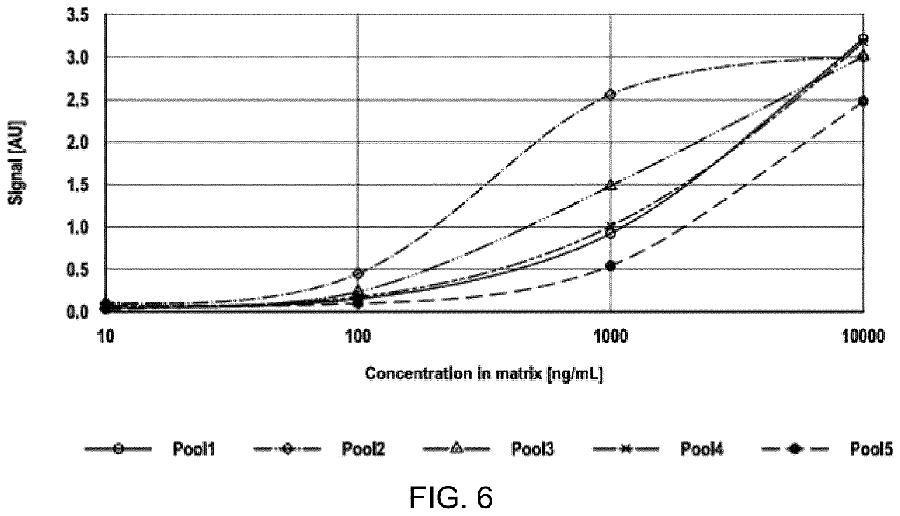

All pools were tested in a bridging ADA assay. Pool 2, 3 and 4 were mixed. To mix pools together is a common procedure using chemically bound proteins. Before mixing them, an ADA ELISA assay was performed and the pools with the highest signals were combined. This leads to a higher yield. The results are shown in FIG. 6. It can be seen that with all pools compared to the monomeric antibody an increased signal can be obtained. This allows for the first time the use as positive control and calibration standard in a bridging ADA-determining immunoassay.

It can further be seen that the most prominent improvement can be obtained with high degrees of cross-linking, i.e. Pools 2 and 3.

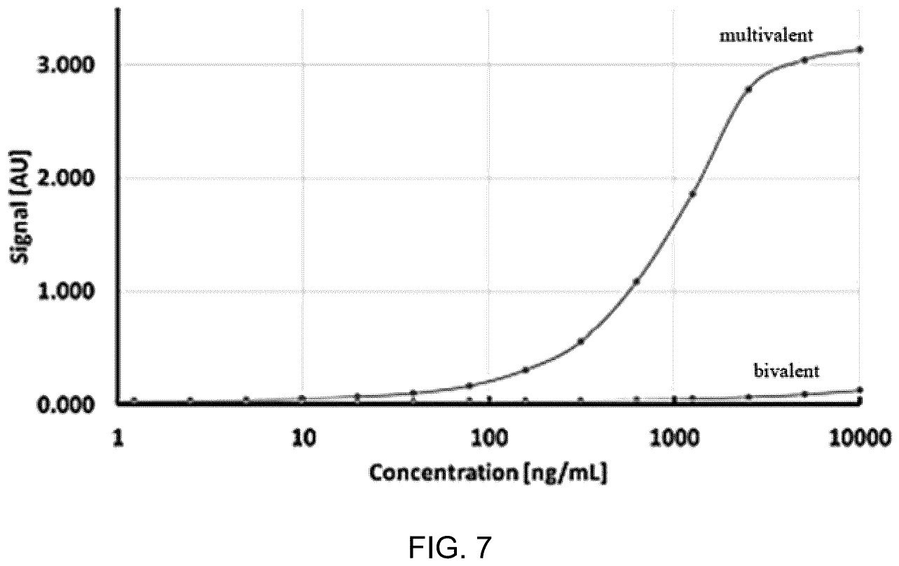

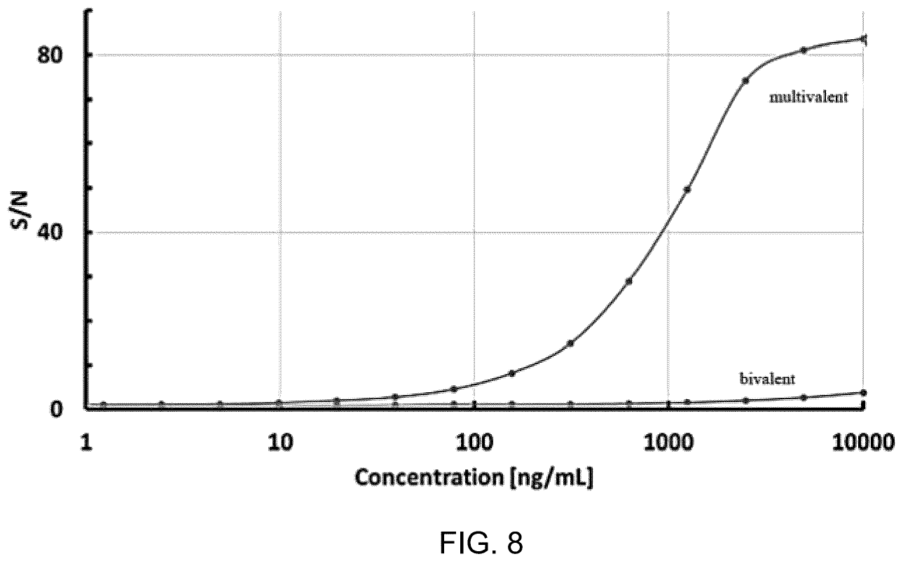

In FIGS. 7 and 8 the monomeric, bivalent anti-PG antibody clone 1.7.24 and the multivalent anti-PG antibody clone 1.7.24 according to the current invention are shown as direct 1:1 comparison with respect to signal and signal-to-noise ratio. In both cases a substantial improvement can be seen. For example, the signal-to-noise ratio (S/N) for the multivalent anti-PG antibody clone 1.7.24 is 4.5 at a concentration of 80 ng/ml and 83.6 at a concentration of 1000 ng/ml. The bivalent anti-PG antibody clone 1.7.24 has a S/N of only 3.8 at a concentration of 1000 ng/mL.

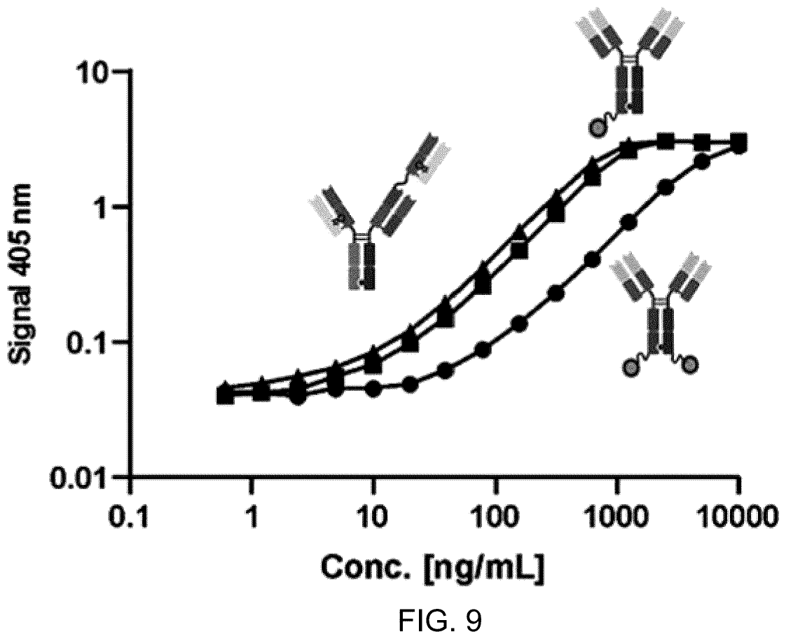

Further, the multivalent anti-PG antibody has been tested with different drug antibodies in different formats. Independent of the format the signal improvement can be seen (see FIG. 9).

In a second example, the invention is exemplified in the following according to FIG. 4B with a recombinantly produced tetravalent form of an anti-PG antibody, i.e. an antibody specifically binding to a human Fc-region of the IgG subclass with the P329G alteration. This is presented solely to exemplify the invention and shall not be construed as limitation. The true scope is set forth in the appended claims.

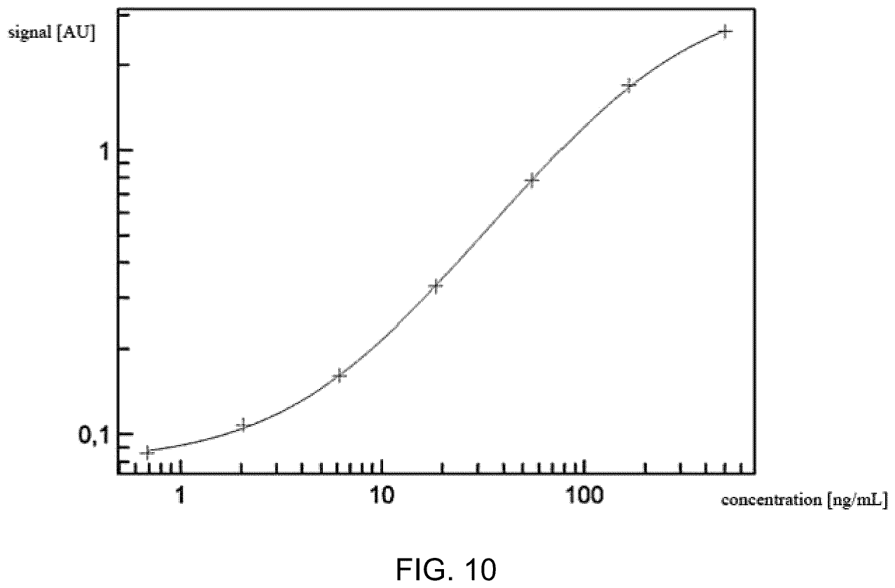

In more detail, the tetravalent anti-PG antibody was used as calibration standard in a bridging immunoassay, with the same but differently derivatized drug antibodies Fc-regions as capture and tracer antibody. The results are shown in FIG. 10.

In a third example, the invention is exemplified in the following with a recombinantly produced IgM variant of an anti-PG antibody, i.e. an antibody specifically binding to a human Fc-region of the IgG subclass with the P329G alteration. This is presented solely to exemplify the invention and shall not be construed as limitation. The true scope is set forth in the appended claims.

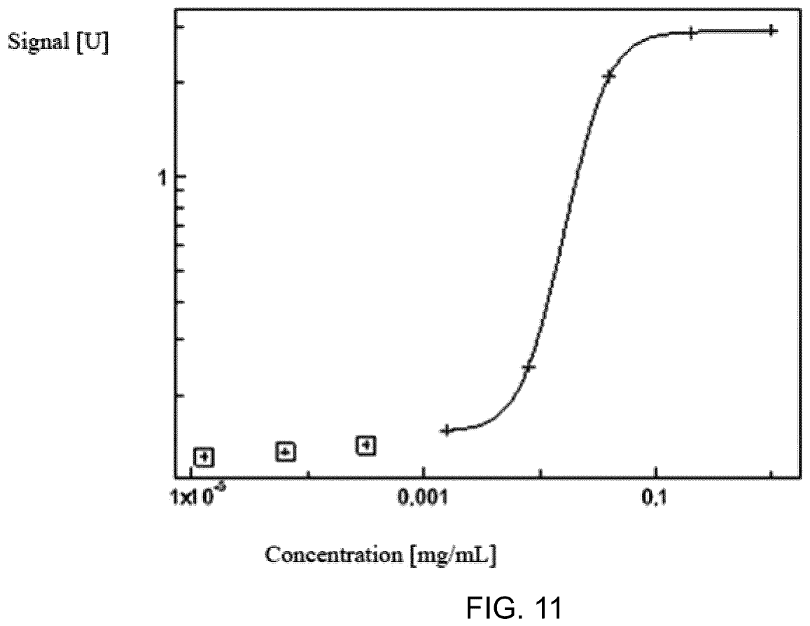

In more detail, the IgM variant anti-PG antibody was used as calibration standard in a bridging immunoassay, with the Fc-region of as capture reagent and the complete drug in TCB format as tracer antibody. The results are shown in FIG. 11.

### A. Exemplary Anti-Variant Fc-Region Antibodies

In the bridging assay, ADAs are both captured and detected by the differentially labelled drug antibodies. However, the bridging assay is able to detect ADA of various Ig-subtypes including IgM and is applicable to all kinds of therapeutic antibodies (Mire-Sluis, A. R., et al., J. Immunol. Meth. 289 (2004) 1-16 (2004): Geng, D., et al., J. Pharm. Biomed. Anal. 39 (2005) 364-375). In the bridging assay, complexes of ADA and drug antibody are detected, independent of the binding region of the therapeutic antibody. In addition, the assay according to the invention is especially suited for drug antibodies bearing the P329G modification within the Fc-region. For this group of drug antibodies, the assay represents a generic approach and can easily be applied.

To summarize, the assay as reported herein offers the possibility for robust and sensitive detection of ADA against Fc-region modified drug antibodies. In combination with the standard bridging assay, it can be used to characterize an immune response in more detail.

The assay as reported herein is a generic approach and is applicable for all drug antibodies, e.g. those with a Pro329Gly substitution, i.e. with prevented/abolished FcγR binding. The assay according to the invention detects ADA and is based on two differently labelled drug antibodies, (i) a bi-labelled drug antibody and (ii) dig-labelled drug antibody.

Moreover, several other Fc modifications than the PG-substitution have been identified that affect the affinity of drug antibodies to both Fc receptors and complement and, as consequence, alter their functional profile (Moore, G. L., et al., MAbs 2 (2010) 181-189; Richards, J. O., et al., Mol. Cancer. Ther. 7 (2008) 2517-2527: Lazar, G. A., et al., Proc. Natl. Acad. Sci. USA 103 (2010) 4005-4010; Schlothauer, T., et al., Prot. Eng. Des. Sel. 29 (2016) 457-466).

The principle of the assay as reported herein can be transferred to a wide range of Fc-region alterations provided that these allow the generation of specific antibodies.

To summarize, the combination of the conventional bridging assay with the multivalent antibody according to the current invention helps to characterize the immunogenicity profile of drug antibodies with suppressed or altered Fc effector function.

One aspect according to the invention is a multivalent an antibody specifically binding to an immunoglobulin Fc-region of the human IgG1 subclass comprising one, two, three or four amino acid alterations compared to a wild-type Fc-region of the human IgG1 subclass.

Specifically binding denotes that the antibody binds to a wild-type immunoglobulin Fc-region of the human IgG1 subclass with a K_{D}-value of 10⁻⁸ mol/l or more.

One aspect according to the invention is a multivalent antibody specifically binding to an immunoglobulin Fc-region of the human IgG1 subclass comprising at position 329 the amino acid residue glycine (numbering according to Kabat EU index), wherein the multivalent antibody comprises at least three binding site comprising
(1)
   (a) a HVR-H1 comprising the amino acid sequence of SEQ ID NO: 09;
   (b) a HVR-H2 comprising the amino acid sequence of SEQ ID NO: 12;
   (c) a HVR-H3 comprising the amino acid sequence of SEQ ID NO: 16;
   (d) a HVR-L1 comprising the amino acid sequence of SEQ ID NO: 23;
   (e) a HVR-L2 comprising the amino acid sequence of SEQ ID NO: 26; and
   (f) a HVR-L3 comprising the amino acid sequence of SEQ ID NO: 28; or
(2)
   (a) a HVR-H1 comprising the amino acid sequence of SEQ ID NO: 10;
   (b) a HVR-H2 comprising the amino acid sequence of SEQ ID NO: 13;
   (c) a HVR-H3 comprising the amino acid sequence of SEQ ID NO: 17;
   (d) a HVR-L1 comprising the amino acid sequence of SEQ ID NO: 23 or 24;
   (e) a HVR-L2 comprising the amino acid sequence of SEQ ID NO: 26; and
   (f) a HVR-L3 comprising the amino acid sequence of SEQ ID NO: 29; or
(3)
   (a) a HVR-H1 comprising the amino acid sequence of SEQ ID NO: 10;
   (b) a HVR-H2 comprising the amino acid sequence of SEQ ID NO: 14;
   (c) a HVR-H3 comprising the amino acid sequence of SEQ ID NO: 18;
   (d) a HVR-L1 comprising the amino acid sequence of SEQ ID NO: 23 or 24;
   (e) a HVR-L2 comprising the amino acid sequence of SEQ ID NO: 26; and
   (f) a HVR-L3 comprising the amino acid sequence of SEQ ID NO: 30; or
(4)
   (a) a HVR-H1 comprising the amino acid sequence of SEQ ID NO: 20;
   (b) a HVR-H2 comprising the amino acid sequence of SEQ ID NO: 21;
   (c) a HVR-H3 comprising the amino acid sequence of SEQ ID NO: 22;
   (d) a HVR-L1 comprising the amino acid sequence of SEQ ID NO: 32;
   (e) a HVR-L2 comprising the amino acid sequence of SEQ ID NO: 34; and
   (f) a HVR-L3 comprising the amino acid sequence of SEQ ID NO: 35; or
(5)
   a mixture or combination of any one of (1) and/or (2) and/or (3) and/or (4).

In one preferred embodiment of all aspects and embodiments of the invention, the multivalent antibody specifically binds to an immunoglobulin Fc-region of the human IgG1 subclass comprising at position 329 the amino acid residue glycine and at positions 234 and 235 the amino acid residue alanine (numbering according to Kabat EU index). In one preferred embodiment, the multivalent antibody is a tetravalent antibody or a multimeric form of the bivalent antibody or a multimeric (Fab′)₂ fragment of the bivalent antibody.

Divalent (Fab′)₂ fragments have two antigen-binding sites that are linked to each other by disulfide bonds. Two individual Fab fragments are produced when a full-length, Y-shaped antibody is digested with papain. A (Fab′)₂ fragment, which retains a portion of the hinge region, is produced by pepsin digestion of IgG or IgM antibodies.

In certain embodiments of all aspects and embodiments of the invention, the multimer is a dimer, a trimer, a tetramer, a pentamer, a hexamer, a heptamer, an octamer, a nonamer, or a decamer.

One aspect according to the invention is the use of a multivalent antibody according to the invention as positive control in a (bridging) immunoassay.

One aspect according to the current invention is the use of a multivalent antibody according to the invention as calibration standard in a (bridging) immunoassay. In certain embodiments, the use is for the generation of a calibration function. In one preferred embodiment, the calibration function is for quantitative determination of anti-drug antibodies against a drug antibody, wherein the anti-drug antibodies bind to at least one amino acid residue in the Fc-region of the drug antibody that is altered compared to the wild-type Fc-region.

In certain embodiments of all aspects and embodiments of the invention, the (bridging) immunoassay is for the determination of anti-drug antibodies against a drug antibody, wherein the anti-drug antibodies bind to the Fc-region of the drug antibody. In certain embodiments, the anti-drug antibody binds to at least one amino acid residue in the Fc-region of the drug antibody that is altered compared to the wild-type Fc-region.

In certain embodiments of all aspects and embodiments of the invention, the immunoassay is a bridging ELISA.

In certain embodiments of all aspects and embodiments of the invention, the drug antibody comprises an immunoglobulin Fc-region of the human IgG1 subclass comprising at position 329 the amino acid residue glycine (numbering according to Kabat EU index).

In one preferred embodiment of all aspects and embodiments of the invention, the drug antibody comprises an immunoglobulin Fc-region of the human IgG1 subclass comprising at position 329 the amino acid residue glycine and at positions 234 and 235 the amino acid residue alanine (numbering according to Kabat EU index).

In certain embodiments of all aspects and embodiments of the invention, the drug antibody comprises an immunoglobulin Fc-region of the human IgG1 subclass comprising at position 253, 310 and 435 the amino acid residue alanine (numbering according to Kabat EU index).

In one preferred embodiment of all aspects and embodiments of the invention, the immunoassay is a bridging immunoassay for the determination of anti-drug antibodies comprising as capture and as tracer antibody the drug antibody.

One aspect according to the invention is an immunoassay for the determination of the presence and/or amount of anti-drug antibodies in a (serum containing) sample,
wherein the anti-drug antibodies bind to at least one amino acid residue in the Fc-region of the drug antibody that is altered compared to the wild-type Fc-region,
wherein the immunoassay comprises as capture and as tracer antibody the drug antibody,
characterized in that
the multivalent antibody according to the invention is used as positive control or as calibration standard in the immunoassay.

In certain embodiments of all aspects and embodiments of the invention, the use as calibration standard is for the generation of a calibration function. In one preferred embodiment, the calibration function is for quantitative determination of anti-drug antibodies against a drug antibody, wherein the anti-drug antibodies bind to at least one amino acid residue in the Fc-region of the drug antibody that is altered compared to the wild-type Fc-region.

In certain embodiments of all aspects and embodiments of the invention, the antibody specifically binding to a variant immunoglobulin Fc-region of the human IgG1 subclass is a monoclonal antibody.

In certain embodiments of all aspects and embodiments of the invention, the drug antibody is a human, humanized, or chimeric antibody.

One aspect is a method of producing a multimer according to the invention comprising chemically cross-linking a full-length antibody specifically binding to a variant Fc-region using N-succinimidyl-3-acetylthiopropionate (SATP) and maleimidohexanoyl-N-hydroxysuccinimide (MHS).

One aspect is a method of producing a multimer according to the invention comprising chemically cross-linking a full-length antibody specifically binding to an immunoglobulin Fc-region of the human IgG1 subclass comprising at position 329 the amino acid residue glycine (numbering according to Kabat EU index), wherein the antibody comprises
(1)
   (a) a HVR-H1 comprising the amino acid sequence of SEQ ID NO: 09;
   (b) a HVR-H2 comprising the amino acid sequence of SEQ ID NO: 12;
   (c) a HVR-H3 comprising the amino acid sequence of SEQ ID NO: 16;
   (d) a HVR-L1 comprising the amino acid sequence of SEQ ID NO: 23;
   (e) a HVR-L2 comprising the amino acid sequence of SEQ ID NO: 26; and
   (f) a HVR-L3 comprising the amino acid sequence of SEQ ID NO: 28; or
(2)
   (a) a HVR-H1 comprising the amino acid sequence of SEQ ID NO: 10;
   (b) a HVR-H2 comprising the amino acid sequence of SEQ ID NO: 13;
   (c) a HVR-H3 comprising the amino acid sequence of SEQ ID NO: 17;
   (d) a HVR-L1 comprising the amino acid sequence of SEQ ID NO: 23 or 24;
   (e) a HVR-L2 comprising the amino acid sequence of SEQ ID NO: 26; and
   (f) a HVR-L3 comprising the amino acid sequence of SEQ ID NO: 29; or
(3)
   (a) a HVR-H1 comprising the amino acid sequence of SEQ ID NO: 10;
   (b) a HVR-H2 comprising the amino acid sequence of SEQ ID NO: 14;
   (c) a HVR-H3 comprising the amino acid sequence of SEQ ID NO: 18;
   (d) a HVR-L1 comprising the amino acid sequence of SEQ ID NO: 23 or 24;
   (e) a HVR-L2 comprising the amino acid sequence of SEQ ID NO: 26; and
   (f) a HVR-L3 comprising the amino acid sequence of SEQ ID NO: 30; or
(4)
   (a) a HVR-H1 comprising the amino acid sequence of SEQ ID NO: 20;
   (b) a HVR-H2 comprising the amino acid sequence of SEQ ID NO: 21;
   (c) a HVR-H3 comprising the amino acid sequence of SEQ ID NO: 22;
   (d) a HVR-L1 comprising the amino acid sequence of SEQ ID NO: 32;
   (e) a HVR-L2 comprising the amino acid sequence of SEQ ID NO: 34; and
   (f) a HVR-L3 comprising the amino acid sequence of SEQ ID NO: 35; or
(5)
   a mixture or combination of any one of (1) to (4);
   using N-succinimidyl-3-acetylthiopropionate (SATP) and maleimidohexanoyl-N-hydroxysuccinimide (MHS).

In one preferred embodiment of all aspects and embodiments of the invention, the anti-variant Fc-region antibody comprises
(1)
   (a) a HVR-H1 comprising the amino acid sequence of SEQ ID NO: 09;
   (b) a HVR-H2 comprising the amino acid sequence of SEQ ID NO: 12;
   (c) a HVR-H3 comprising the amino acid sequence of SEQ ID NO: 16;
   (d) a HVR-L1 comprising the amino acid sequence of SEQ ID NO: 23;
   (e) a HVR-L2 comprising the amino acid sequence of SEQ ID NO: 26; and
   (f) a HVR-L3 comprising the amino acid sequence of SEQ ID NO: 28; or
(2)
   (a) a HVR-H1 comprising the amino acid sequence of SEQ ID NO: 10;
   (b) a HVR-H2 comprising the amino acid sequence of SEQ ID NO: 13;
   (c) a HVR-H3 comprising the amino acid sequence of SEQ ID NO: 17;
   (d) a HVR-L1 comprising the amino acid sequence of SEQ ID NO: 23 or 24;
   (e) a HVR-L2 comprising the amino acid sequence of SEQ ID NO: 26; and
   (f) a HVR-L3 comprising the amino acid sequence of SEQ ID NO: 29; or
(3)
   (a) a HVR-H1 comprising the amino acid sequence of SEQ ID NO: 10;
   (b) a HVR-H2 comprising the amino acid sequence of SEQ ID NO: 14;
   (c) a HVR-H3 comprising the amino acid sequence of SEQ ID NO: 18;
   (d) a HVR-L1 comprising the amino acid sequence of SEQ ID NO: 23 or 24;
   (e) a HVR-L2 comprising the amino acid sequence of SEQ ID NO: 26; and
   (f) a HVR-L3 comprising the amino acid sequence of SEQ ID NO: 30; or
(4)
   (a) a HVR-H1 comprising the amino acid sequence of SEQ ID NO: 20;
   (b) a HVR-H2 comprising the amino acid sequence of SEQ ID NO: 21;
   (c) a HVR-H3 comprising the amino acid sequence of SEQ ID NO: 22;
   (d) a HVR-L1 comprising the amino acid sequence of SEQ ID NO: 32;
   (e) a HVR-L2 comprising the amino acid sequence of SEQ ID NO: 34; and
   (f) a HVR-L3 comprising the amino acid sequence of SEQ ID NO: 35; or
(5)
   a mixture of any one of (1) to (4).

The antibodies used in the current invention have the following sequences (HVRs determined according to Kabat):

| | | | HVR- | HVR- | HVR- | HVR- | HVR- | HVR- |
|---|---|---|---|---|---|---|---|---|
| antibody | VH | VL | H1 | H2 | H3 | L1 | L2 | L3 |
| 1.1.3 | 01 | 02 | 09 | 12 | 16 | 23 | 26 | 28 |
| 1.3.17 | 03 | 04 | 10 | 13 | 17 | 24 | 26 | 29 |
| 1.7.24 | 37 | 08 | 10 | 14 | 18 | 23 | 26 | 30 |
| 1.6.22 | 36 | 06 | 20 | 21 | 22 | 32 | 34 | 35 |

SEQ ID NO: 36 (SEQ ID NO: 05 without signal
sequence):
E VQLVESGGDL VKPGGSLKLS CAASGFTFSS YGMSWVRQTP
DKRLEWVATI SSGGSYIYYP DSVKGRFTIS RDNAKNTLYL
QMSSLKSEDT AMYYCARLGM ITTGYAMDYW GQGTSVTVSS
SEQ ID NO: 06:
DVLMTQTPLS LPVSLGDQAS ISCRSSQTIV HSTGHTYLEW
FLQKPGQSPK LLIYKVSNRF SGVPDRFSGS GSGTDFTLKI
SRVEAEDLGV YYCFQGSHVP YTFGGGTKLE IK
SEQ ID NO: 37 (SEQ ID NO: 07 without signal
sequence):
EV KLLESGGGLV QPGGSLKLSC AASGFDFSRY WMNWVRQAPG
KGLEWIGEIT PDSSTINYTP SLKDKFIISR DNAKNTLYLQ
MIKVRSEDTA LYYCVRPYDY GAWFASWGQG TLVTVSA
SEQ ID NO: 08:
QAVVTQESAL TTSPGETVTL TCRSSTGAVT TSNYANWVQE
KPDHLFTGLI GGTNKRAPGV PARFSGSLIG DKAALTITGA
QTEDEAIYFC ALWYSNHWVF GGGTKLTVL

In certain embodiments of all aspects and embodiments of the invention, the anti-variant (human) Fc-region antibody used for the preparation of the multivalent antibody according to the invention (anti-AAA antibody).
specifically binds to an epitope on the variant (human) Fc-region of the IgG1 subclass comprising the amino acid residue (A) 253, (A) 310 and (A) 435 (numbering according to Kabat EU index),
specifically binds to a variant (human) Fc-region of the IgG1 subclass that has an alanine amino acid residue at positions 253, 310 and 435 (numbering according to Kabat EU index),
does not (specifically) bind to the wild-type (human) Fc-region of the IgG1 subclass that has an isoleucine amino acid residue at position 253 and a histidine amino acid residue at position 310 and a histidine amino acid residue at position 435 (numbering according to Kabat EU index),
does not (specifically) bind to the (human) Fc-region of the IgG1 subclass that has an isoleucine amino acid residue at position 253 and a histidine amino acid residue at position 310 and a histidine amino acid residue at position 435 and a glycine amino acid residue at position 329 and an alanine amino acid residue at position 234 and an alanine amino acid residue at position 235 (numbering according to Kabat), and
does not (specifically) bind to the variant (human) Fc-region of the IgG1 subclass that has an isoleucine amino acid residue at position 253 and a histidine amino acid residue at position 310 and a histidine amino acid residue at position 435 and a proline amino acid residue at position 329 and a leucine amino acid residue at position 234 and a leucine amino acid residue at position 235 (numbering according to Kabat).

The term “does not (specifically) bind to” denotes that in an assay in which the binding is determined the results obtained is not significantly different from the result obtained with a sample not comprising the antibody in question, i.e. a blank sample or a buffer sample.

In certain embodiments of all aspects and embodiments of the invention, the variant (human) Fc-region is an Fc-region of the human IgG1 or IgG4 subclass with the mutations I253A, H310A and H435A (numbering according to Kabat EU index).

In certain embodiments of all aspects and embodiments of the invention, the anti-Fc-region antibody that specifically binds to an Fc-region of the IgG1 subclass comprising at positions 253, 310 and 435 (numbering according to Kabat EU index) each the amino acid residue alanine used for the generation of the multimer according to the invention comprises at least one, two, three, four, five, or six HVRs selected from (a) a HVR-H1 comprising the amino acid sequence of SEQ ID NO: 09 or 10; (b) a HVR-H2 comprising the amino acid sequence of SEQ ID NO: 12, 13 or 14: (c) a HVR-H3 comprising the amino acid sequence of SEQ ID NO: 16, 17 or 18: (d) a HVR-L1 comprising the amino acid sequence of SEQ ID NO: 23 or 24: (e) a HVR-L2 comprising the amino acid sequence of SEQ ID NO: 26; and (f) a HVR-L3 comprising the amino acid sequence of SEQ ID NO: 28, 29 or 30.

In certain embodiments of all aspects and embodiments of the invention, the antibody used to generate the multivalent antibody of the invention comprises (a) a VH domain comprising (i) a HVR-H1 comprising the amino acid sequence of SEQ ID NO: 09 or 10, (ii) a HVR-H2 comprising the amino acid sequence of SEQ ID NO: 12 or 13 or 14, and (iii) a HVR-H3 comprising an amino acid sequence selected from SEQ ID NO: 16, 17 or 18; and (b) a VL domain comprising (i) a HVR-L1 comprising the amino acid sequence of SEQ ID NO: 23 or 24, (ii) HVR-L2 comprising the amino acid sequence of SEQ ID NO: 26, and (c) HVR-L3 comprising the amino acid sequence of SEQ ID NO: 28, 29 or 30.

In certain embodiments of all aspects and embodiments of the invention, the antibody used to generate the multivalent antibody according to the invention comprises (a) a HVR-H1 comprising the amino acid sequence of SEQ ID NO: 09; (b) a HVR-H2 comprising the amino acid sequence of SEQ ID NO: 12: (c) a HVR-H3 comprising the amino acid sequence of SEQ ID NO 16: (d) a HVR-L1 comprising the amino acid sequence of SEQ ID NO: 23: (e) a HVR-L2 comprising the amino acid sequence of SEQ ID NO: 26; and (f) HVR-L3 comprising an amino acid sequence selected from SEQ ID NO: 28.

In certain embodiments of all aspects and embodiments of the invention, the antibody used to generate the multivalent antibody according to the invention comprises (a) a HVR-H1 comprising the amino acid sequence of SEQ ID NO: 10; (b) a HVR-H2 comprising the amino acid sequence of SEQ ID NO: 13: (c) a HVR-H3 comprising the amino acid sequence of SEQ ID NO 17: (d) a HVR-L1 comprising the amino acid sequence of SEQ ID NO: 23: (e) a HVR-L2 comprising the amino acid sequence of SEQ ID NO: 26; and (f) HVR-L3 comprising an amino acid sequence selected from SEQ ID NO: 29.

In certain embodiments of all aspects and embodiments of the invention, the antibody used to generate the multivalent antibody according to the invention comprises (a) a HVR-H1 comprising the amino acid sequence of SEQ ID NO: 10; (b) a HVR-H2 comprising the amino acid sequence of SEQ ID NO: 14: (c) a HVR-H3 comprising the amino acid sequence of SEQ ID NO 18: (d) a HVR-L1 comprising the amino acid sequence of SEQ ID NO: 23: (e) a HVR-L2 comprising the amino acid sequence of SEQ ID NO: 26; and (f) HVR-L3 comprising an amino acid sequence selected from SEQ ID NO: 30.

In certain embodiments of all aspects and embodiments of the invention, the antibody used to generate the multivalent antibody according to the invention comprises any one or more amino acid are substituted at the following HVR positions:
in HVR-H1 (SEQ ID NO: 11): position 5;
in HVR-H2 (SEQ ID NO: 15): positions 3, 7, 8, 11, 12;
in HVR-H3 (SEQ ID NO: 19): positions 2, 10;
in HVR-L1 (SEQ ID NO: 25): positions 3, 14;
in HVR-L2 (SEQ ID NO: 27): positions 4; and
in HVR-L3 (SEQ ID NO: 31): positions 1, 6.

In certain embodiments, the substitutions are conservative substitutions, as provided herein. In certain embodiments, any one or more of the following amino acid residues (any one either alone or in combination independently of each other) may be present in any combination:
in HVR-H1 (SEQ ID NO: 11): at position 5 a neutral hydrophilic amino acid residue selected from the group of amino acid residues consisting of S, T, N, and Q;
in HVR-H2 (SEQ ID NO: 15): at position 3 a neutral hydrophilic or acidic amino acid residue selected from the group of amino acid residues consisting of S, T, N, Q, D and E, at position 7 a neutral hydrophilic or basic amino acid residue selected from the group of amino acid residues consisting of S, T, N, Q, H, K, and R, at position 8 a neutral hydrophilic amino acid residue or a residue that influence chain orientation selected from the group of amino acid residues consisting of S, T, N, Q, G, and P, at position 11 a neutral hydrophilic or aromatic amino acid residue or a residue that influence chain orientation selected from the group of amino acid residues consisting of S, T, N, Q, G, P, W, Y, and F, at position 12 a neutral hydrophilic amino acid residue or a residue that influence chain orientation selected from the group of amino acid residues consisting of S, T, N, Q, G, and P;
in HVR-H3 (SEQ ID NO: 19): at position 2 a hydrophobic or aromatic amino acid residue selected from the group of amino acid residues consisting of M, A, V, L, I, W, Y, and F, at position 10 a neutral hydrophilic or aromatic amino acid residue selected from the group of amino acid residues consisting of S, T, N, Q, W, Y, and F;
in HVR-L1 (SEQ ID NO: 25): at position 3 a neutral hydrophilic amino acid residue selected from the group of amino acid residues consisting of S, T, N, and Q, at position 14 a neutral hydrophilic or acidic amino acid residue selected from the group of amino acid residues consisting of S, T, N, Q, D, and E;
in HVR-L2 (SEQ ID NO: 27): at position 4 an acidic or basic amino acid residue selected from the group of amino acid residues consisting of E, D, H, K, and R; and
in HVR-L3 (SEQ ID NO: 31): at position 1 a hydrophobic amino acid residue selected from the group of amino acid residues consisting of M, A, V, L, and I, at position 6 a neutral hydrophilic or acidic amino acid residue selected from the group of amino acid residues consisting of S, T, N, Q, D, and E.

All possible combinations of the above substitutions are encompassed by the consensus sequences of SEQ ID NO: 11, 15, 19, 25, 27, and 31.

SEQ ID NO: 07 is the murine sequence of the heavy chain variable domain including the signal peptide of 18 amino acid residues at the N-terminus. SEQ ID NO: 37 is derived from SEQ ID NO: 07 by deleting the signal sequence.

SEQ ID NO: 02 and SEQ ID NO: 04, respectively, are murine sequences of the light chain variable domains each including the signal peptide of 19 amino acid residues at the N-terminus.

In certain embodiments of all aspects and embodiments of the invention, the antibody used to generate the multivalent antibody according to the invention comprises a heavy chain variable domain amino acid sequence derived from SEQ ID NO: 01 and a light chain variable domain amino acid sequence derived from SEQ ID NO: 02, and the humanized antibody has the same binding specificity as a chimeric or murine antibody that contains as heavy chain variable domain the amino acid sequence of SEQ ID NO: 01 and as light chain variable domain the amino acid sequence of SEQ ID NO: 02.

In certain embodiments of all aspects and embodiments of the invention, the antibody used to generate the multivalent antibody according to the invention comprises a heavy chain variable domain amino acid sequence derived from SEQ ID NO: 03 and a light chain variable domain amino acid sequence derived from SEQ ID NO: 04, and the humanized antibody has the same binding specificity as a chimeric or murine antibody that contains as heavy chain variable domain the amino acid sequence of SEQ ID NO: 03 and as light chain variable domain the amino acid sequence of SEQ ID NO: 04.

In certain embodiments of all aspects and embodiments of the invention, the antibody used to generate the multivalent antibody according to the invention comprises a heavy chain variable domain amino acid sequence derived from SEQ ID NO: 07 and a light chain variable domain amino acid sequence derived from SEQ ID NO: 08, and the humanized antibody has the same binding specificity as a chimeric or murine antibody that contains as heavy chain variable domain the amino acid sequence of SEQ ID NO: 07 and as light chain variable domain the amino acid sequence of SEQ ID NO: 08.

In certain embodiments of all aspects and embodiments of the invention, the antibody used to generate the multivalent antibody according to the invention comprises a heavy chain variable domain (VH) sequence having at least 90%, 91%, 92%, 93%, 94%, 95%, 96%, 97%, 98%, 99%, or 100% sequence identity to the amino acid sequence of any one of SEQ ID NO: 01, 03 and 37. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-variant (human) Fc-region antibody comprising that sequence retains the ability to bind to the variant (human) Fc-region. In certain embodiments, a total of 1 to 10 amino acids have been substituted, inserted and/or deleted in any one of SEQ ID NO: 01, 03 and 37. In certain embodiments, substitutions, insertions, or deletions occur in regions outside the HVRs (i.e., in the FRs). Optionally, the anti-variant (human) Fc-region antibody comprises the VH sequence as in any one of SEQ ID NO: 01, 03 and 37, including post-translational modifications of that sequence.

In certain embodiments of all aspects and embodiments of the invention, the antibody used to generate the multivalent antibody according to the invention comprises a light chain variable domain (VL) having at least 90%, 91%, 92%, 93%, 94%, 95%, 96%, 97%, 98%, 99%, or 100% sequence identity to the amino acid sequence of any one of SEQ ID NO: 02, 04 or 08. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-variant (human) Fc-region antibody comprising that sequence retains the ability to bind to the variant (human) Fc-region. In certain embodiments, a total of 1 to 10 amino acids have been substituted, inserted and/or deleted in SEQ ID NO: 02, 04 or 08. In certain embodiments, the substitutions, insertions, or deletions occur in regions outside the HVRs (i.e., in the FRs). Optionally, the anti-variant (human) Fc-region antibody comprises the VL sequence of SEQ ID NO: 02, 04 or 08, including post-translational modifications of that sequence.

In certain embodiments of all aspects and embodiments of the invention, the antibody used to generate the multivalent antibody according to the invention comprises a VH as in any of the embodiments provided above, and a VL as in any of the embodiments provided above. In certain embodiments, the antibody comprises (i) the VH and VL sequences in SEQ ID NO: 01 and SEQ ID NO: 02, or (ii) the VH and VL sequences in SEQ ID NO: 03 and SEQ ID NO: 04, respectively, or (iii) the VH and VL sequences in SEQ ID NO: 37 and SEQ ID NO: 08, including post-translational modifications of those sequences.

In certain embodiments of all aspects and embodiments of the invention, the anti-variant (human) Fc-region antibody used for the preparation of the multivalent antibody according to the invention as reported herein (anti-PG antibody)
specifically binds to an epitope on the variant (human) Fc-region of the IgG1 subclass comprising the amino acid residue (s (A) 234, (A) 235 and) (G) 329 (numbering according to Kabat EU index),
specifically binds to a variant (human) Fc-region of the IgG1 subclass that has (an alanine amino acid residue at position 234 and 235, and) a glycine amino acid residue at position 329 (numbering according to Kabat EU index),
specifically binds to a variant (human) Fc-region of the IgG1 subclass that has an alanine amino acid residue at position 234 and 235, a glycine amino acid residue at position 329 and an isoleucine amino acid residue at position 253 and a histidine amino acid residue at position 310 and a histidine amino acid residue at position 435 (numbering according to Kabat),
specifically binds to a variant (human) Fc-region of the IgG1 subclass that has an alanine amino acid residue at position 234, 235, 253, 310 and 435, and a glycine amino acid residue at position 329 (numbering according to Kabat),
does not (specifically) bind to the wild-type (human) Fc-region of the IgG1 subclass that has (a leucine amino acid residue at position 234 and 235, and) a proline amino acid residue at position 329 (numbering according to Kabat),
does not (specifically) bind to the wild-type (human) Fc-region of the IgG1 subclass that has a leucine amino acid residue at position 234 and 235, and a proline amino acid residue at position 329 and an isoleucine amino acid residue at position 253 and a histidine amino acid residue at position 310 and a histidine amino acid residue at position 435 (numbering according to Kabat), and
does not (specifically) bind to the variant (human) Fc-region of the IgG1 subclass that has a leucine amino acid residue at position 234 and 235, and a proline amino acid residue at position 329 and an alanine amino acid residue at position 253 and an alanine amino acid residue at position 310 and an alanine amino acid residue at position 435 (numbering according to Kabat).

As the immunization performed for the generation of the anti-PG antibody was performed with human IgG1 bearing the P329G, L234A and L235A Fc-region substitutions, it was expected to obtain an antibody specifically binding to these amino acid residues. Surprisingly, the antibody obtained specifically binds to human IgG1 and Fc-region fragments only having the P329G mutation independently of the presence or absence of the L234A and L235A mutation, whereas human wt-IgG1 and human IgG1 with the mutations L234A and L235A were not bound. Thus, the anti-PG antibody used to generate the multimer according to the invention is specific for the single P329G-substitution in the Fc-region of human IgG1.

In certain embodiments of all aspects and embodiments of the invention, the variant (human) Fc-region is an Fc-region of the human IgG1 or IgG4 subclass with the mutation P329G (numbering according to Kabat EU index).

In certain embodiments of all aspects and embodiments of the invention, the antibody used to generate the multivalent antibody according to the invention is an anti-Fc-region antibody that specifically binds to an Fc-region of the IgG1 subclass comprising at position 329 the amino acid residue glycine (and optionally at positions 234 and 235 the amino acid residue alanine) (numbering according to Kabat EU index) comprising at least one, two, three, four, five, or six HVRs selected from (a) a HVR-H1 comprising the amino acid sequence of SEQ ID NO: 20; (b) a HVR-H2 comprising the amino acid sequence of SEQ ID NO: 21: (c) a HVR-H3 comprising the amino acid sequence of SEQ ID NO: 22: (d) a HVR-L1 comprising the amino acid sequence of SEQ ID NO: 32: (e) a HVR-L2 comprising the amino acid sequence of SEQ ID NO: 34; and (f) a HVR-L3 comprising the amino acid sequence of SEQ ID NO: 35.

In certain embodiments of all aspects and embodiments of the invention, the antibody used to generate the multivalent antibody according to the invention comprises (a) a VH domain comprising (i) a HVR-H1 comprising the amino acid sequence of SEQ ID NO: 20, (ii) a HVR-H2 comprising the amino acid sequence of SEQ ID NO: 21, and (iii) a HVR-H3 comprising an amino acid sequence selected from SEQ ID NO: 22; and (b) a VL domain comprising (i) a HVR-L1 comprising the amino acid sequence of SEQ ID NO: 32, (ii) HVR-L2 comprising the amino acid sequence of SEQ ID NO: 34, and (c) HVR-L3 comprising the amino acid sequence of SEQ ID NO: 35.

In certain embodiments of all aspects and embodiments of the invention, the antibody used to generate the multivalent antibody according to the invention comprises (a) a HVR-H1 comprising the amino acid sequence of SEQ ID NO: 20; (b) a HVR-H2 comprising the amino acid sequence of SEQ ID NO: 21: (c) a HVR-H3 comprising the amino acid sequence of SEQ ID NO 22: (d) a HVR-L1 comprising the amino acid sequence of SEQ ID NO: 32: (e) a HVR-L2 comprising the amino acid sequence of SEQ ID NO: 34; and (f) HVR-L3 comprising an amino acid sequence selected from SEQ ID NO: 35.

In certain embodiments of all aspects and embodiments of the invention, in the antibody used to generate the multivalent antibody according to the invention the amino acid of an anti-variant (human) Fc-region antibody is substituted at the following HVR position:
in HVR-L1 (SEQ ID NO: 33): position 9.

In certain embodiments, the substitutions are conservative substitutions, as provided herein. In certain embodiments, any one or more of the following amino acid residues (any one either alone or in combination independently of each other) may be present in any combination:
in HVR-L1 (SEQ ID NO: 33): at position 9 a neutral hydrophilic amino acid residue or a residue that influences chain orientation selected from the group of amino acid residues consisting of S, T, N, Q, G and P.

All possible combinations of the above substitutions are encompassed by the consensus sequence of SEQ ID NO: 33.

SEQ ID NO: 05 is the murine sequence of the heavy chain variable domain including the signal peptide of 19 amino acid residues at the N-terminus. SEQ ID NO: 36 is derived from SEQ ID NO: 05 by deleting the signal sequence.

In certain embodiments of all aspects and embodiments of the invention, the antibody used to generate the multivalent antibody according to the invention comprises a heavy chain variable domain amino acid sequence derived from SEQ ID NO: 05 and a light chain variable domain amino acid sequence derived from SEQ ID NO: 06, and the humanized antibody has the same binding specificity as a chimeric or murine antibody that contains as heavy chain variable domain the amino acid sequence of SEQ ID NO: 05 and as light chain variable domain the amino acid sequence of SEQ ID NO: 06.

In certain embodiments of all aspects and embodiments of the invention, the antibody used to generate the multivalent antibody according to the invention comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 36. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-variant (human) Fc-region antibody comprising that sequence retains the ability to bind to the variant (human) Fc-region. In certain embodiments, a total of 1 to 10 amino acids have been substituted, inserted and/or deleted in SEQ ID NO: 36. In certain embodiments, substitutions, insertions, or deletions occur in regions outside the HVRs (i.e., in the FRs). Optionally, the anti-variant (human) Fc-region antibody comprises the VH sequence as in SEQ ID NO: 36, including post-translational modifications of that sequence.

In certain embodiments of all aspects and embodiments of the invention, the antibody used to generate the multivalent antibody according to the invention comprises a light chain variable domain (VL) having at least 90%, 91%, 92%, 93%, 94%, 95%, 96%, 97%, 98%, 99%, or 100% sequence identity to the amino acid sequence of SEQ ID NO: 06. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-variant (human) Fc-region antibody comprising that sequence retains the ability to bind to the variant (human) Fc-region. In certain embodiments, a total of 1 to 10 amino acids have been substituted, inserted and/or deleted in SEQ ID NO: 06. In certain embodiments, the substitutions, insertions, or deletions occur in regions outside the HVRs (i.e., in the FRs). Optionally, the anti-variant (human) Fc-region antibody comprises the VL sequence of SEQ ID NO: 06, including post-translational modifications of that sequence.)

In certain embodiments of all aspects and embodiments of the invention, the antibody used to generate the multivalent antibody according to the invention comprises a VH as in any of the embodiments provided above, and a VL as in any of the embodiments provided above. In certain embodiments, the antibody comprises the VH and VL sequences in SEQ ID NO: 36 and SEQ ID NO: 06, including post-translational modifications of those sequences.

In certain embodiments of all aspects and embodiments of the invention, the anti-variant (human) Fc-region antibody according to any of the above embodiments is a monoclonal antibody, including a chimeric, humanized or human antibody. In certain embodiments, the anti-variant (human) Fc-region antibody is an antibody fragment, e.g., a diabody, or F(ab′)₂ fragment. In certain embodiments, the antibody is a full-length antibody, e.g., an intact antibody of the human IgG1 subclass or other antibody class or isotype as defined herein.

One aspect of the current invention is an antibody comprising four or six binding sites specifically binding to an immunoglobulin Fc-region of the human IgG1 subclass comprising one, two, three or four amino acid changes compared to a wild-type Fc-region of the human IgG1 subclass.

One aspect of the current invention is an antibody multimer comprising at least two covalently linked
i) bivalent, full length antibodies each comprising two binding sites specifically binding to an immunoglobulin Fc-region of the human IgG1 subclass comprising one, two, three or four amino acid changes compared to a wild-type Fc-region of the human IgG1 subclass, or
ii) (Fab′)2 fragments of a bivalent, full-length antibody each comprising two binding sites specifically binding to an immunoglobulin Fc-region of the human IgG1 subclass comprising one, two, three or four amino acid changes compared to a wild-type Fc-region of the human IgG1 subclass.

One aspect of the current invention is an antibody or antibody multimer according to the invention, wherein the binding sites specifically binding to an immunoglobulin Fc-region of the human IgG1 subclass comprising one, two, three or four amino acid changes compared to a wild-type Fc-region of the human IgG1 subclass are binding sites specifically binding to an immunoglobulin Fc-region of the human IgG1 subclass comprising at position 329 the amino acid residue glycine (numbering according to Kabat EU index).

One aspect of the current invention is an antibody or antibody multimer according to the invention, wherein each of the binding sites comprises independently of each other either
(1)
   (a) a HVR-H1 comprising the amino acid sequence of SEQ ID NO: 09;
   (b) a HVR-H2 comprising the amino acid sequence of SEQ ID NO: 12;
   (c) a HVR-H3 comprising the amino acid sequence of SEQ ID NO: 16;
   (d) a HVR-L1 comprising the amino acid sequence of SEQ ID NO: 23;
   (e) a HVR-L2 comprising the amino acid sequence of SEQ ID NO: 26; and
   (f) a HVR-L3 comprising the amino acid sequence of SEQ ID NO: 28; or
(2)
   (a) a HVR-H1 comprising the amino acid sequence of SEQ ID NO: 10;
   (b) a HVR-H2 comprising the amino acid sequence of SEQ ID NO: 13;
   (c) a HVR-H3 comprising the amino acid sequence of SEQ ID NO: 17;
   (d) a HVR-L1 comprising the amino acid sequence of SEQ ID NO: 23;
   (e) a HVR-L2 comprising the amino acid sequence of SEQ ID NO: 26; and
   (f) a HVR-L3 comprising the amino acid sequence of SEQ ID NO: 29; or
(3)
   (a) a HVR-H1 comprising the amino acid sequence of SEQ ID NO: 10;
   (b) a HVR-H2 comprising the amino acid sequence of SEQ ID NO: 14;
   (c) a HVR-H3 comprising the amino acid sequence of SEQ ID NO: 18;
   (d) a HVR-L1 comprising the amino acid sequence of SEQ ID NO: 23;
   (e) a HVR-L2 comprising the amino acid sequence of SEQ ID NO: 26; and
   (f) a HVR-L3 comprising the amino acid sequence of SEQ ID NO: 30; or
(4)
   (a) a HVR-H1 comprising the amino acid sequence of SEQ ID NO: 20;
   (b) a HVR-H2 comprising the amino acid sequence of SEQ ID NO: 21;
   (c) a HVR-H3 comprising the amino acid sequence of SEQ ID NO: 22;
   (d) a HVR-L1 comprising the amino acid sequence of SEQ ID NO: 32;
   (e) a HVR-L2 comprising the amino acid sequence of SEQ ID NO: 34; and
   (f) a HVR-L3 comprising the amino acid sequence of SEQ ID NO: 35; or
(5)
   a mixture of any one of (1) to (4).

One aspect of the current invention is an antibody or antibody multimer according to the invention, wherein the binding sites specifically binding to an immunoglobulin Fc-region of the human IgG1 subclass comprising one, two, three or four amino acid changes compared to a wild-type Fc-region of the human IgG1 subclass are binding sites specifically binding to an immunoglobulin Fc-region of the human IgG1 subclass comprising at position 329 the amino acid residue glycine and at positions 234 and 235 the amino acid residue alanine (numbering according to Kabat EU index).

One aspect of the current invention is an antibody multimer according to the invention, wherein the multimer is a dimer, a trimer, a tetramer, a pentamer, a hexamer, a heptamer, an octamer, a nonamer, or a decamer.

One aspect of the current invention is the use of an antibody or antibody multimer according to the invention as positive control in an in vitro (bridging) immunoassay.

One aspect of the current invention is the use of an antibody or antibody multimer according to the invention as a standard in an in vitro (bridging) immunoassay.

One aspect of the current invention is the use of an antibody or antibody multimer according to the invention, wherein the use is for the generation of a calibration function for quantitative determination of anti-drug antibodies against a drug antibody, wherein the anti-drug antibodies bind to one or more amino acid residue(s) in the Fc-region of the drug antibody that is altered compared to a wild-type Fc-region.

One aspect of the invention is an immunoassay for the determination of the presence and/or amount of anti-drug antibodies in a (serum containing) sample,
wherein the anti-drug antibodies bind to at least one amino acid residue in the Fc-region of the drug antibody that is altered compared to a wild-type Fc-region,
wherein the immunoassay comprises as capture and as tracer antibody the drug antibody,
characterized in that
the antibody or antibody multimer according to the invention is used as positive control or as calibration standard in the immunoassay.

One aspect of the current invention is an immunoassay using the antibody or antibody multimer according to the invention, wherein the immunoassay is a bridging ELISA.

One aspect of the current invention is an immunoassay, wherein using the antibody or antibody multimer according to the invention is used as calibration standard and is used for the generation of a calibration function, which is for quantitative determination of anti-drug antibodies against a drug antibody.

One aspect of the invention is a method of producing an antibody multimer according to the invention by chemical conjugation of bivalent, full length antibodies each comprising two binding sites specifically binding to an immunoglobulin Fc-region of the human IgG1 subclass comprising one, two, three or four amino acid changes compared to a wild-type Fc-region of the human IgG1 subclass using N-succinimidyl-3-acetylthiopropionate (SATP) and maleimidohexanoyl-N-hydroxysuccinimide (MHS).

One aspect of the current invention is a method for producing an antibody multimer according to the invention, wherein the multimer is a multimer of full-length antibodies.

One aspect of the invention is a method of producing an antibody multimer comprising
chemically cross-linking a full-length antibody specifically binding to an immunoglobulin Fc-region of the human IgG1 subclass comprising at position 329 the amino acid residue glycine (numbering according to Kabat EU index),
   wherein the antibody comprises
(1)
   (a) a HVR-H1 comprising the amino acid sequence of SEQ ID NO: 09;
   (b) a HVR-H2 comprising the amino acid sequence of SEQ ID NO: 12;
   (c) a HVR-H3 comprising the amino acid sequence of SEQ ID NO: 16;
   (d) a HVR-L1 comprising the amino acid sequence of SEQ ID NO: 23;
   (e) a HVR-L2 comprising the amino acid sequence of SEQ ID NO: 26; and
   (f) a HVR-L3 comprising the amino acid sequence of SEQ ID NO: 28; or
(2)
   (a) a HVR-H1 comprising the amino acid sequence of SEQ ID NO: 10;
   (b) a HVR-H2 comprising the amino acid sequence of SEQ ID NO: 13;
   (c) a HVR-H3 comprising the amino acid sequence of SEQ ID NO: 17:
   (d) a HVR-L1 comprising the amino acid sequence of SEQ ID NO: 23;
   (e) a HVR-L2 comprising the amino acid sequence of SEQ ID NO: 26; and
   (f) a HVR-L3 comprising the amino acid sequence of SEQ ID NO: 29; or
(3)
   (a) a HVR-H1 comprising the amino acid sequence of SEQ ID NO: 10;
   (b) a HVR-H2 comprising the amino acid sequence of SEQ ID NO: 14;
   (c) a HVR-H3 comprising the amino acid sequence of SEQ ID NO: 18;
   (d) a HVR-L1 comprising the amino acid sequence of SEQ ID NO: 23;
   (e) a HVR-L2 comprising the amino acid sequence of SEQ ID NO: 26; and
   (f) a HVR-L3 comprising the amino acid sequence of SEQ ID NO: 30;
   using N-succinimidyl-3-acetylthiopropionate (SATP) and maleimidohexanoyl-N-hydroxysuccinimide (MHS).

An antibody comprising binding sites specifically binding to an immunoglobulin Fc-region of the human IgG1 subclass comprising one, two, three or four amino acid changes compared to a wild-type Fc-region of the human IgG1 subclass can be generated using any method known in the art.

For example, antibodies may be prepared by administering an immunogene comprising at least the respective variant part of the Fc-region to an experimental animal. A suitable construct for presenting the variant Fc-region is reported, e.g., in WO 2012/150320.

Antibodies can also be made by hybridoma-based methods. For example, human myeloma and mouse-human heteromyeloma cell lines for the production of human monoclonal antibodies have been described. (See, e.g., Kozbor J. Immunol., 133:3001 (1984); Brodeur et al., Monoclonal Antibody Production Techniques and Applications, pp. 51-63 (Marcel Dekker, Inc., New York, 1987); and Boerner et al., J. Immunol., 147:86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26 (4): 265-268 (2006) (describing human-human hybridomas). Human hybridoma technology (Trioma technology) is also described in Vollmers and Brandlein, Histology and Histopathology, 20 (3): 927-937 (2005) and Vollmers and Brandlein, Methods and Findings in Experimental and Clinical Pharmacology, 27 (3): 185-91 (2005).

Antibodies may also be generated by isolating variable domain sequences selected from human-derived phage display libraries. Such variable domain sequences may then be combined with a desired human constant domain.

Techniques for selecting antibodies from antibody libraries are known in the art.

In certain embodiments, the anti-variant (human) Fc-region antibody according to any of the above embodiments may incorporate any of the features, singly or in combination, as described in Sections 1-3 below:

### 1. Antibody Fragments

In certain embodiments, an antibody used to generate the multivalent antibody according to the invention is a bivalent antibody fragment. Bivalent antibody fragments include, but are not limited to, F(ab′)₂, and other fragments described below as long as these are bivalent. For a review of certain antibody fragments, see Hudson, P. J. et al., Nat. Med. 9 (2003) 129-134. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 0 404 097: WO 1993/01161; Hudson, P. J. et al., Nat. Med. 9 (2003) 129-134; and Holliger, P. et al., Proc. Natl. Acad. Sci. USA 90 (1993) 6444-6448. Triabodies and tetrabodies are also described in Hudson, P. J., et al., Nat. Med. 9 (20039 129-134).

Antibody fragments can be made by various techniques, including but not limited to proteolytic digestion of an intact antibody as well as production by recombinant host cells (e.g. E. coli or phage), as described herein.

### 2. Chimeric and Humanized Antibodies

In certain embodiments, an antibody used to generate the multivalent antibody according to the invention is a chimeric antibody. Certain chimeric antibodies are described, e.g., in U.S. Pat. No. 4,816,567; and Morrison, S. L. et al., Proc. Natl. Acad. Sci. USA 81 (1984) 6851-6855). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro, J. C. and Fransson, J., Front. Biosci. 13 (2008) 1619-1633, and are further described, e.g., in Riechmann, I. et al., Nature 332 (1988) 323-329; Queen, C. et al., Proc. Natl. Acad. Sci. USA 86 (1989) 10029-10033: U.S. Pat. Nos. 5,821,337, 7,527,791, 6,982,321, and 7,087,409; Kashmiri, S. V. et al., Methods 36 (2005) 25-34 (describing specificity determining region (SDR) grafting); Padlan, E. A., Mol. Immunol. 28 (1991) 489-498 (describing “resurfacing”); Dall'Acqua, W. F. et al., Methods 36 (2005) 43-60 (describing “FR shuffling”); and Osbourn, J. et al., Methods 36 (2005) 61-68 and Klimka, A. et al., Br. J. Cancer 83 (2000) 252-260 (describing the “guided selection” approach to FR shuffling).

Human framework regions that may be used for humanization include but are not limited to: framework regions selected using the “best-fit” method (see, e.g., Sims, M. J. et al., J. Immunol. 151 (1993) 2296-2308; framework regions derived from the consensus sequence of human antibodies of a particular subgroup of light or heavy chain variable regions (see, e.g., Carter, P. et al., Proc. Natl. Acad. Sci. USA 89 (1992) 4285-4289; and Presta, L. G. et al., J. Immunol. 151 (1993) 2623-2632); human mature (somatically mutated) framework regions or human germline framework regions (see, e.g., Almagro, J. C. and Fransson, J., Front. Biosci. 13 (2008) 1619-1633); and framework regions derived from screening FR libraries (see, e.g., Baca, M. et al., J. Biol. Chem. 272 (1997) 10678-10684 and Rosok, M. J. et al., J. Biol. Chem. 271 (19969 22611-22618).

### 3. Antibody Variants

In certain embodiments, amino acid sequence variants of the antibodies used to generate the multivalent antibody according to the invention provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen binding.

### a) Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table 1 under the heading of “preferred substitutions”. More changes that are substantial are provided in the following Table under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity-matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, P. S., Methods Mol. Biol. 207 (2008) 179-196), and/or residues that contact antigen, with the resulting variant VH or VL being tested for binding affinity. Affinity maturation by constructing and reselecting from secondary libraries has been described, e.g., in Hoogenboom, H. R. et al. in Methods in Molecular Biology 178 (2002) 1-37. In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham, B. C. and Wells, J. A., Science 244 (1989) 1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

### b) Glycosylation Variants

In certain embodiments, an antibody used to generate the multivalent antibody according to the invention is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc-region, the carbohydrate attached thereto may be altered. Native antibodies produced by mammalian cells typically comprise a branched, biantennary oligosaccharide that is generally attached by an N-linkage to Asn297 of the CH2 domain of the Fc-region. See, e.g., Wright, A. and Morrison, S. L., TIBTECH 15 (1997) 26-32. The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In certain embodiments, the antibody used to generate the multivalent antibody according to the invention is an antibody variant having a carbohydrate structure that lacks fucose attached (directly or indirectly) to an Fc-region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc-region (EU numbering of Fc-region residues); however, Asn297 may also be located about +3 amino acids upstream or downstream of position 297, i.e., between positions 294 and 300, due to minor sequence variations in antibodies. Such fucosylation variants may have improved ADCC function. See, e.g., US 2003/0157108; US 2004/0093621. Examples of publications related to “defucosylated” or “fucose-deficient” antibody variants include: US 2003/0157108: WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO 2005/053742; WO 2002/031140; Okazaki, A. et al., J. Mol. Biol. 336 (2004) 1239-1249; Yamane-Ohnuki, N. et al., Biotech. Bioeng. 87 (2004) 614-622. Examples of cell lines capable of producing defucosylated antibodies include Lec13 CHO cells deficient in protein fucosylation (Ripka, J. et al., Arch. Biochem. Biophys. 249 (1986) 533-545; US 2003/0157108; and WO 2004/056312, especially at Example 11), and knockout cell lines, such as alpha-1,6-fucosyltransferase gene, FUT8, knockout CHO cells (see, e.g., Yamane-Ohnuki, N. et al., Biotech. Bioeng. 87 (2004) 614-622; Kanda, Y. et al., Biotechnol. Bioeng. 94 (2006) 680-688; WO 2003/085107).

Antibodies variants are further encompassed with bisected oligosaccharides, e.g., in which a biantennary oligosaccharide attached to the Fc-region of the antibody is bisected by GlcNAc. Such antibody variants may have reduced fucosylation and/or improved ADCC function. Examples of such antibody variants are described, e.g., in WO 2003/011878: U.S. Pat. No. 6,602,684; and US 2005/0123546. Antibody variants with at least one galactose residue in the oligosaccharide attached to the Fc-region are also provided. Such antibody variants may have improved CDC function. Such antibody variants are described, e.g., in WO 1997/30087; WO 1998/58964; and WO 1999/22764.

### c) Fc-Region Variants

In certain embodiments, one or more amino acid modifications may be introduced into the Fc-region of an antibody used to generate the multivalent antibody according to the invention, thereby generating an Fc-region variant. The Fc-region variant may comprise a human Fc-region sequence (e.g., a human IgG1, IgG2, IgG3 or IgG4 Fc-region) comprising an amino acid modification (e.g. a substitution) at one or more amino acid positions.

In certain embodiments, the antibody used to generate the multivalent antibody according to the invention is an antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch, J. V. and Kinet, J. P., Annu. Rev. Immunol. 9 (1991) 457-492. Non-limiting examples of in vitro assays to assess ADCC activity of a molecule of interest is described in U.S. Pat. No. 5,500,362 (see, e.g. Hellstrom, I. et al., Proc. Natl. Acad. Sci. USA 83 (1986) 7059-7063; and Hellstrom, I. et al., Proc. Natl. Acad. Sci. USA 82 (1985) 1499-1502): U.S. Pat. No. 5,821,337 (see Bruggemann, M. et al., J. Exp. Med. 166 (1987) 1351-1361). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes, R. et al., Proc. Natl. Acad. Sci. USA 95 (1998) 652-656. C1q binding assays may also be carried out to confirm that the antibody is unable to bind C1q and hence lacks CDC activity (see, e.g., C1q and C3c binding ELISA in WO 2006/029879 and WO 2005/100402). To assess complement activation, a CDC assay may be performed (see, for example, Gazzano-Santoro, H. et al., J. Immunol. Methods 202 (1996) 163-171; Cragg, M. S. et al., Blood 101 (2003) 1045-1052; and Cragg, M. S. and M. J. Glennie, Blood 103 (2004) 2738-2743). FcRn binding and in vivo clearance/half-life determinations can also be performed using methods known in the art (see, e.g., Petkova, S. B. et al., Int. Immunol. 18 (2006:1759-1769).

Antibodies with reduced effector function include those with substitution of one or more of Fc-region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields, R. L. et al., J. Biol. Chem. 276 (2001) 6591-6604)

In certain embodiments, an antibody variant comprises an Fc-region with one or more amino acid substitutions that improve ADCC, e.g., substitutions at positions 298, 333, and/or 334 of the Fc-region (EU numbering of residues).

In certain embodiments, alterations are made in the Fc-region that result in altered (i.e., either improved or diminished) C1q binding and/or Complement Dependent Cytotoxicity (CDC), e.g., as described in U.S. Pat. No. 6,194,551, WO 99/51642, and Idusogie, E. E. et al., J. Immunol. 164 (2000) 4178-4184.

Antibodies with increased half-lives and improved binding to the neonatal Fc receptor (FcRn), which is responsible for the transfer of maternal IgGs to the fetus (Guyer, R. L. et al., J. Immunol. 117 (1976) 587-593, and Kim, J. K. et al., J. Immunol. 24 (1994) 2429-2434), are described in US 2005/0014934. Those antibodies comprise an Fc-region with one or more substitutions therein which improve binding of the Fc-region to FcRn. Such Fc variants include those with substitutions at one or more of Fc-region residues: 238, 256, 265, 272, 286, 303, 305, 307, 311, 312, 317, 340, 356, 360, 362, 376, 378, 380, 382, 413, 424 or 434, e.g., substitution of Fc-region residue 434 (U.S. Pat. No. 7,371,826). See also Duncan, A. R. and Winter, G., Nature 322 (1988) 738-740;

U.S. Pat. Nos. 5,648,260; 5,624,821; and WO 94/29351 concerning other examples of Fc-region variants.

The C-terminus of the heavy chain of the antibody used to generate the multimer according to the invention can be a complete C-terminus ending with the amino acid residues PGK. The C-terminus of the heavy chain can be a shortened C-terminus in which one or two of the C-terminal amino acid residues have been removed. In one preferred embodiment the C-terminus of the heavy chain is a shortened C-terminus ending PG.

### d) Cysteine Engineered Antibody Variants

In certain embodiments, it may be desirable to create cysteine-engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc-region. Cysteine engineered antibodies may be generated as described, e.g., in U.S. Pat. No. 7,521,541.

### B. Recombinant Methods and Compositions

Antibodies may be produced using recombinant methods and compositions, e.g., as described in U.S. Pat. No. 4,816,567. In certain embodiments, isolated nucleic acid encoding an anti-variant (human) Fc-region antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In certain embodiments, the host cell is eukaryotic, e.g. a Chinese Hamster Ovary (CHO) cell or lymphoid cell (e.g., Y0, NS0, PS20 cell). In certain embodiments, a method of making an anti-variant (human) Fc-region antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-variant (human) Fc-region antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, K. A., In: Methods in Molecular Biology, Vol. 248, Lo, B. K. C. (ed.), Humana Press, Totowa, NJ (2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, T. U., Nat. Biotech. 22 (2004) 1409-1414; and Li, H. et al., Nat. Biotech. 24 (2006) 210-215.

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham, F. L. et al., J. Gen Virol. 36 (1977) 59-74); baby hamster kidney cells (BHK); mouse sertoli cells (TM4 cells as described, e.g., in Mather, J. P., Biol. Reprod. 23 (1980) 243-252); monkey kidney cells (CV1); African green monkey kidney cells (VERO-76); human cervical carcinoma cells (HELA); canine kidney cells (MDCK: buffalo rat liver cells (BRL 3A); human lung cells (W138); human liver cells (Hep G2): mouse mammary tumor (MMT 060562): TRI cells, as described, e.g., in Mather, J. P. et al., Annals N.Y. Acad. Sci. 383 (1982) 44-68; MRC 5 cells; and FS4 cells. Other useful mammalian host cell lines include Chinese hamster ovary (CHO) cells, including DHFR-CHO cells (Urlaub, G. et al., Proc. Natl. Acad. Sci. USA 77 (1980) 4216-4220); and myeloma cell lines such as Y0, NS0 and Sp2/0. For a review of certain mammalian host cell lines suitable for antibody production, see, e.g., Yazaki, P. and Wu, A. M., Methods in Molecular Biology, Vol. 248, Lo, B. K. C. (ed.), Humana Press, Totowa, NJ (2004), pp. 255-268.

### C. Assays

Multivalent anti-variant (human) Fc-region antibodies according to the invention can be used in various assays known in the art.

The multivalent antibodies according to the invention are especially useful if a therapeutic antibody comprising the respective mutations in the Fc-region or an anti-drug antibody against such a therapeutic antibody has to be detected, e.g. in a sample.

In certain embodiment of all aspects and embodiments according to the invention, the drug antibody comprises
i) the mutations P329G or P329G, L234A and L235A, and/or
ii) the mutations I253A, H310A and H435A.

In certain embodiment of all aspects and embodiments according to the invention, the antibody comprising the respective mutations is an antibody comprising
i) the mutations P329G or P329G, L234A and L235A, and/or
ii) the mutations I253A, H310A and H435A.

One aspect according to the invention is the use of a multivalent antibody according to the invention in an (antigen bridging) immunoassay either as positive control or as (calibration) standard for the determination of anti-drug antibodies against/specifically binding to a therapeutic antibody comprising the respective mutations in the Fc-region (i.e. an antibody comprising the respective mutations in the Fc-region) (in a sample). The respective other reagent required for detection and for capture is the therapeutic antibody, which has been derivatized, immobilized or labelled accordingly.

This assay is applicable to any non-human serum. In certain embodiments, the use is for the determination in a serum sample of a non-human experimental animal.

Such an assay has a lower limit of quantification (threshold) of below 100 μg/mL, e.g. of 40-80 pg/mL in 10% cynomolgus serum (assay concentration).

One aspect according to the invention is the use of the multivalent antibody according to the invention in an immunoassay for the determination of an anti-drug antibody against a therapeutic antibody whereby the therapeutic antibody comprises the respective mutations in the Fc-region (in a sample).

In certain embodiments of all aspects and embodiments of the invention, the drug antibody is used as a capture antibody. The capture antibody is in certain embodiments immobilized to a solid surface. This solid surface is in one preferred embodiment (the wall or the bottom or both of) a well of a multi-well plate.

In certain embodiments of all aspects and embodiments of the invention, the drug antibody is used as a tracer antibody. For detecting the tracer antibody is conjugated to a suitable label.

In certain embodiments of all aspects and embodiments of the invention, the sample is obtained from an experimental animal selected from the members of the families of marmosets and tamarins, old world monkeys, dwarf and mouse lemurs, gibbons and lesser apes, true lemurs, as well as crossings thereof or from a human. In certain embodiments, the sample is obtained from a rhesus monkey, or a marmoset monkey, or a baboon monkey, or a cynomolgus monkey, or a human. In certain embodiments, the experimental animal is a macaca or macaque monkey. In certain embodiments, the sample is obtained from a cynomolgus monkey or a rhesus monkey or a human.

In certain embodiments of all aspects and embodiments of the invention, the immunoassay is a sandwich immunoassay.

In certain embodiments of all aspects and embodiment of the invention, the conjugation of the drug (therapeutic) antibody to its conjugation partner is performed by chemically binding via N-terminal and/or ε-amino groups (lysine), ε-amino groups of different lysins, carboxy-, sulfhydryl-, hydroxyl- and/or phenolic functional groups of the amino acid backbone of the antibody and/or sugar alcohol groups of the carbohydrate structure of the antibody. In certain embodiments, the capture antibody is immobilized via a specific binding pair. In one preferred embodiment, the capture antibody is conjugated to biotin and immobilization is performed via immobilized avidin or streptavidin. In certain embodiments, the tracer antibody is conjugated to the detectable label via a specific binding pair. In one preferred embodiment, the tracer antibody is conjugated to digoxygenin and linking to the detectable label is performed via an antibody against digoxygenin. In certain embodiments, the drug antibody is a human or a humanized antibody. In certain embodiments, the human or humanized antibody is a monoclonal antibody.

One aspect according to the invention is a method for determining the correct/proper function of a sandwich/bridging immunoassay for the determination of anti-drug antibodies against a therapeutic antibody, which has a modified (effector function silent) Fc-region, or an Fc-region thereof (in a sample) comprising the steps of:
a) incubating a multivalent antibody according to the invention with the drug (therapeutic) antibody or an Fc-region (fragment) thereof that has been immobilized on a solid surface to form an dimeric complex,
b) incubating said dimeric complex with the drug (therapeutic) antibody conjugated to a detectable label to form a ternary complex, and
c) determining the correct/proper function of the sandwich/bridging immunoassay if the ternary complex is formed in step b)/the ternary complex formed in step b) can be detected.

One aspect according to the invention is a method for calibrating a sandwich/bridging immunoassay for the determination of anti-drug antibodies against a therapeutic antibody, which has a modified (effector function silent) Fc-region, or an Fc-region thereof (in a sample) comprising the steps of:
a) incubating a multivalent antibody according to the invention separately at at least two different concentrations with the drug (therapeutic) antibody or an Fc-region (fragment) thereof that has been immobilized on a solid surface to a dimeric complex,
b) incubating each of said dimeric complexes separately with the drug (therapeutic) antibody conjugated to a detectable label to form ternary complexes,
c) determining the amount of each of the ternary complexes formed in step c) by determining the amount of the detectable label, and
d) calculating a calibration curve based on the amounts determined in step c) and thereby calibrating the sandwich/bridging immunoassay.

One aspect according to the current invention is an anti-drug antibody immunoassay for the determination of the presence of an anti-drug antibody against an Fc-receptor binding suppressed human or humanized drug antibody (i.e. an antibody comprising the respective mutations in the Fc-region) (in a sample)/the Fc-region of an Fc-receptor binding suppressed human or humanized drug antibody (i.e. an antibody comprising the respective mutations in the Fc-region) (in a sample), wherein the method comprises the following steps in the following order:
a) incubating a solid phase on which the Fc-receptor binding suppressed human or humanized drug antibody or an Fc-region fragment thereof has been immobilized with a sample comprising mammalian blood serum (so that a solid-phase-bound drug antibody-anti-drug antibody complex is formed),
b) incubating the solid phase (to which the drug antibody-anti-drug antibody complex formed in step a) is bound) with the drug antibody or an Fc-region fragment thereof conjugated to a detectable label, and
c) determining the formation of a solid-phase-bound complex in step b) by determining the presence of the detectable label and thereby determining the presence of an anti-drug antibody against an Fc-receptor binding suppressed human or humanized drug antibody in the sample,
whereby the correct function of the immunoassay has been determined using a multivalent antibody according to the current invention.

One aspect according to the current invention is an anti-drug antibody immunoassay for the determination of the presence of an anti-drug antibody against an Fc-receptor binding suppressed human or humanized drug antibody (i.e. an antibody comprising the respective mutations in the Fc-region) (in a sample)/the Fc-region of an Fc-receptor binding suppressed human or humanized drug antibody (i.e. an antibody comprising the respective mutations in the Fc-region) (in a sample), wherein the method comprises the following steps in the following order:
a) incubating a solid phase on which the Fc-receptor binding suppressed human or humanized drug antibody has been immobilized with a sample comprising mammalian blood serum (so that a solid-phase-bound drug antibody-anti-drug antibody complex is formed),
b) incubating the solid phase (to which the drug antibody-anti-drug antibody complex formed in step a) is bound) with the drug antibody conjugated to a detectable label, and
c) determining the formation of a solid-phase-bound complex in step b) by determining the presence of the detectable label and thereby determining the presence of an anti-drug antibody against an Fc-receptor binding suppressed human or humanized drug antibody in the sample,
whereby a standard/calibration curve for the immunoassay has been determined using a multivalent antibody according to the current invention.

In certain embodiments of all aspects and embodiments according to the invention, each incubating step is followed by the following step:
washing the solid phase to remove unbound compounds.

In certain embodiments of all aspects and embodiments according to the invention, the determination of the presence or the amount of the detectable label is done by:
determining the formation of a solid-phase-bound complex in the previous step by determining the presence of the detectable label and determining the amount of the complex by determining the amount of the determined label.

In certain embodiments of all aspects and embodiments of the invention, the Fc-receptor binding suppressed human or humanized drug antibody is of the human IgG1 or IgG4 subclass.

In certain embodiments of all aspects and embodiments of the invention, the Fc-receptor binding suppressed human or humanized drug antibody is of the human IgG1 subclass and has the mutations L234A, L235A and P329G in both Fc-region polypeptides, or the Fc-receptor binding suppressed human or humanized drug antibody is of the human IgG4 subclass and has the mutations S228P, L235E and P329G in both Fc-region polypeptides (numbering according to the EU numbering system according to Kabat).

In certain embodiments of all aspects and embodiments of the invention, the Fc-receptor binding suppressed human or humanized drug antibody is of the human IgG1 subclass and has the mutations I253A, H310A and H435A in both Fc-region polypeptides (numbering according to the EU numbering system according to Kabat).

In certain embodiments of all aspects and embodiments of the invention, the Fc-receptor binding suppressed human or humanized drug antibody is a bispecific antibody, or a trispecific antibody, or a tetraspecific antibody, or a pentaspecific antibody, or a hexaspecific antibody. In one preferred embodiment, the Fc-receptor binding suppressed human or humanized drug antibody is a bispecific antibody.

In certain embodiments of all aspects and embodiments of the invention, the mammalian blood serum is human blood serum or cynomolgus blood serum or mouse blood serum.

In certain embodiments of all aspects and embodiments of the invention, the presence and/or amount of the label is determined using an enzyme linked color reaction, surface plasmon resonance, electrochemiluminescense, or radioimmunoassay.

In certain embodiments of all aspects and embodiments of the invention, the solid phase is conjugated to a first member of a binding pair and the compound to be immobilized on the solid phase is conjugated to the second member of a binding pair. Such a binding pair (first member/second member) is, in certain embodiments, selected from streptavidin or avidin/biotin, antibody/antigen (see, for example, Hermanson, G. T., et al., Bioconjugate Techniques, Academic Press (1996)), lectin/polysaccharide, steroid/steroid binding protein, hormone/hormone receptor, enzyme/substrate, IgG/Protein A and/or G, etc. In certain embodiments, the compound to be immobilized on the solid phase is conjugated to the second member of the binding pair by chemically binding via N-terminal and/or ε-amino groups (lysine), ε-amino groups of different lysins, carboxy-, sulfhydryl-, hydroxyl- and/or phenolic functional groups of the amino acid backbone of the polypeptide and/or sugar alcohol groups of the carbohydrate structure of the polypeptide.

Such conjugation via different amino groups can be performed by acylation of a part of the ε-amino groups with chemical protecting agents, e.g. by citraconylation, in a first step. In a second step, conjugation is performed via the remaining amino groups. Subsequently citraconylation is removed and the binding partner is immobilized on the solid phase via remaining free amino groups, i.e. the binding partner obtained is immobilized on the solid phase via amino groups that have not been protected by citraconylation. Suitable chemical protecting agents form bonds at unprotected side chain amines and are less stable than and different from those bonds at the N-terminus. Many such chemical protecting agents are known (see for example EP 0 651 761). In certain embodiments, the chemical protecting agents include cyclic dicarboxylic acid anhydrides like maleic or citraconylic acid anhydride.

In one preferred embodiment, the first member of a binding pair is streptavidin and the second member of a binding pair is biotin. In certain embodiments, the solid phase is conjugated to streptavidin and the compound to be immobilized on the solid phase is biotinylated. In certain embodiments, the solid phase is a streptavidin coated paramagnetic bead or a streptavidin coated Sepharose bead or a streptavidin coated well of a multi-well-plate.

In certain embodiments of all aspects and embodiments of the invention, the compound to be conjugated to the solid phase is a mixture comprising at least two compounds that differ in the site at which they are conjugated to biotin and thereby immobilized on the solid phase.

In certain embodiments of all aspects and embodiments according to the invention, the multimerization in the multimer according to the invention is performed by chemically binding via N-terminal and/or ε-amino groups (lysine), ε-amino groups of different lysins, carboxy-, sulfhydryl-, hydroxyl- and/or phenolic functional groups of the amino acid backbone of the polypeptide and/or sugar alcohol groups of the carbohydrate structure of the polypeptide.

Coupling via different amino groups can be performed by acylation of a part of the ε-amino groups with chemical protecting agents, e.g. by citraconylation, in a first step. In a second step, conjugation is performed via the remaining amino groups. Subsequently citraconylation is removed and the binding partner is conjugated to the solid phase via remaining free amino groups, i.e. the binding partner obtained is conjugated to the solid phase via amino groups that have not been protected by citraconylation. Suitable chemical protecting agents form bonds at unprotected side chain amines and are less stable than and different from those bonds at the N-terminus. Many such chemical protecting agents are known (see for example EP 0 651 761). In certain embodiments, the chemical protecting agents include cyclic dicarboxylic acid anhydrides like maleic or citraconylic acid anhydride.

In certain embodiments of all aspects and embodiments of the invention, the drug antibody or an Fc-region thereof is conjugated to the solid phase by passive adsorption. Passive adsorption is, e.g., described by Butler, J. E., in “Solid Phases in Immunoassay” (1996) 205-225 and Diamandis, E. P., and Christopoulos, T. K. (Editors), in “Immunoassay” (1996) Academic Press (San Diego).

The term “drug antibody” denotes an antibody which is tested or has been tested in clinical studies for approval as human therapeutic and which can be administered to an individual for the treatment of a disease. In certain embodiments, the drug antibody is a monoclonal antibody. In a further embodiment, the drug antibody is obtained from a great ape or an animal transformed with a human antibody locus, or is a human monoclonal antibody, or is a humanized monoclonal antibody. In certain embodiments, the drug antibody is a human monoclonal antibody. In certain embodiments, the drug antibody is a humanized monoclonal antibody. Drug antibodies are being used widely for the treatment of various diseases such as oncological diseases (e.g. hematological and solid malignancies including non-Hodgkin's lymphoma, breast cancer, and colorectal cancer), immunological diseases, central nervous diseases, vascular diseases, or infectious diseases.

The term “epitope” denotes a protein determinant capable of specifically binding to an antibody. Epitopes usually consist of chemically active surface groupings of molecules such as amino acids or sugar side chains and usually epitopes have specific three-dimensional structural characteristics, as well as specific charge characteristics. Conformational and non-conformational epitopes are distinguished in that the binding to the former but not the latter is lost in the presence of denaturing solvents.

The principles of different immunoassays are described, for example, by Hage, D. S. (Anal. Chem. 71 (1999) 294R-304R). Lu, B., et al. (Analyst 121 (1996) 29R-32R) report the orientated immobilization of antibodies for the use in immunoassays. Avidin-biotin-mediated immunoassays are reported, for example, by Wilchek, M., and Bayer, E. A., in Methods Enzymol. 184 (1990) 467-469.

Polypeptides and monoclonal antibodies and their constant domains contain a number of reactive amino acid side chains for conjugating to a member of a binding pair, such as a polypeptide/protein, a polymer (e.g. PEG, cellulose or polystyrol), or an enzyme. Chemical reactive groups of amino acids are, for example, amino groups (lysins, alpha-amino groups), thiol groups (cystins, cysteines, and methionins), carboxylic acid groups (aspartic acids, glutamic acids), and sugar-alcoholic groups. Such methods are e.g. described by Aslam M., and Dent, A., in “Bioconjugation”, MacMillan Ref. Ltd. 1999, pages 50-100.

One of the most common reactive groups of polypeptides and antibodies is the aliphatic s-amine of the amino acid lysine. In general, nearly all polypeptides and antibodies contain abundant lysine. Lysine amines are reasonably good nucleophiles above pH 8.0 (pKa=9.18) and therefore react easily and cleanly with a variety of reagents to form stable bonds. Amine-reactive reagents react primarily with lysins and the α-amino groups of proteins. Reactive esters, particularly N-hydroxy-succinimide (NHS) esters, are among the most commonly employed reagents for modification of amine groups. The optimum pH for reaction in an aqueous environment is pH 8.0 to 9.0. Isothiocyanates are amine-modification reagents and form thiourea bonds with proteins. They react with protein amines in aqueous solution (optimally at pH 9.0 to 9.5). Aldehydes react under mild aqueous conditions with aliphatic and aromatic amines, hydrazines, and hydrazides to form an imine intermediate (Schiff's base). A Schiff's base can be selectively reduced with mild or strong reducing agents (such as sodium borohydride or sodium cyanoborohydride) to derive a stable alkyl amine bond. Other reagents that have been used to modify amines are acid anhydrides. For example, diethylenetriaminepentaacetic anhydride (DTPA) is a bifunctional chelating agent that contains two amine-reactive anhydride groups. It can react with N-terminal and ε-amine groups of amino acids to form amide linkages. The anhydride rings open to create multivalent, metal chelating arms able to bind tightly to metals in a coordination complex.

Another common reactive group in polypeptides and antibodies is the thiol residue from the sulfur-containing amino acid cystine and its reduction product cysteine (or half cystine). Cysteine contains a free thiol group, which is more nucleophilic than amines and is generally the most reactive functional group in a protein. Thiols are generally reactive at neutral pH, and therefore can be coupled to other molecules selectively in the presence of amines. Since free sulfhydryl groups are relatively reactive, proteins with these groups often exist with them in their oxidized form as disulfide groups or disulfide bonds. In such proteins, reduction of the disulfide bonds with a reagent such as dithiotreitol (DTT) is required to generate the reactive free thiol. Thiol-reactive reagents are those that will couple to thiol groups on polypeptides, forming thioether-coupled products. These reagents react rapidly at slight acidic to neutral pH and therefore can be reacted selectively in the presence of amine groups. The literature reports the use of several thiolating crosslinking reagents such as Traut's reagent (2-iminothiolane), succinimidyl (acetylthio) acetate (SATA), and sulfosuccinimidyl 6-[3-(2-pyridyldithio) propionamido]hexanoate (Sulfo-LC-SPDP) to provide efficient ways of introducing multiple sulfhydryl groups via reactive amino groups. Haloacetyl derivatives, e.g. iodoacetamides, form thioether bonds and are also reagents for thiol modification. Further useful reagents are maleimides. The reaction of maleimides with thiol-reactive reagents is essentially the same as with iodoacetamides. Maleimides react rapidly at slight acidic to neutral pH.

Another common reactive group in polypeptides and antibodies are carboxylic acids. Polypeptides and antibodies contain carboxylic acid groups at the C-terminal position and within the side chains of aspartic acid and glutamic acid. The relatively low reactivity of carboxylic acids in water usually makes it difficult to use these groups to selectively modify polypeptides and antibodies. When this is done, the carboxylic acid group is usually converted to a reactive ester by the use of a water-soluble carbodiimide and reacted with a nucleophilic reagent such as an amine, hydrazide, or hydrazine. The amine-containing reagent should be weakly basic in order to react selectively with the activated carboxylic acid in the presence of the more highly basic s-amines of lysine to form a stable amide bond. Protein crosslinking can occur when the pH is raised above 8.0.

Sodium periodate can be used to oxidize the alcohol part of a sugar within a carbohydrate moiety attached to an antibody to an aldehyde. Each aldehyde group can be reacted with an amine, hydrazide, or hydrazine as described for carboxylic acids. Since the carbohydrate moiety is predominantly found on the crystallizable fragment (Fc) region of an antibody, conjugation can be achieved through site-directed modification of the carbohydrate away from the antigen-binding site. A Schiff's base intermediate is formed, which can be reduced to an alkyl amine through the reduction of the intermediate with sodium cyanoborohydride (mild and selective) or sodium borohydride (strong) water-soluble reducing agents.

The term “sample” includes, but is not limited to, any quantity of a substance from a living thing or formerly living thing. Such living things include, but are not limited to, humans, mice, monkeys, rats, rabbits, and other animals. In certain embodiments, the sample is obtained from a monkey, especially a cynomolgus monkey, or a rabbit, or a mouse, or rat, or a human. Such substances include, but are not limited to, in certain embodiments, whole blood or serum from an individual, which are the most widely used sources of sample in clinical routine.

The term “solid phase” denotes a non-fluid substance, and includes particles (including microparticles and beads) made from materials such as polymer, metal (paramagnetic, ferromagnetic particles), glass, and ceramic: gel substances such as silica, alumina, and polymer gels: capillaries, which may be made of polymer, metal, glass, and/or ceramic: zeolites and other porous substances: electrodes: microtiter plates: solid strips; and cuvettes, tubes or other spectrometer sample containers. A solid phase component is distinguished from inert solid surfaces in that a “solid phase” contains at least one moiety on its surface, which is intended to interact with a substance in a sample. A solid phase may be a stationary component, such as a tube, strip, cuvette or microtiter plate, or may be non-stationary components, such as beads and microparticles. A variety of microparticles that allow either non-covalent or covalent attachment of proteins and other substances may be used. Such particles include polymer particles such as polystyrene and poly (methyl methacrylate); gold particles such as gold nanoparticles and gold colloids; and ceramic particles such as silica, glass, and metal oxide particles. See for example Martin, C. R., et al., Analytical Chemistry-News & Features, 70 (1998) 322A-327A, or Butler, J. E., Methods 22 (2000) 4-23.

From chromogens (fluorescent or luminescent groups and dyes), enzymes, NMR-active groups, metal particles, or haptens, such as digoxygenin, the detectable label is selected in certain embodiments. The detectable label can also be a photoactivatable crosslinking group, e.g. an azido or an azirine group. Metal chelates that can be detected by electrochemiluminescense are also, in certain embodiments, signal-emitting groups, with particular preference being given to ruthenium chelates, e.g. a ruthenium (bispyridyl)₃²⁺ chelate. Suitable ruthenium labeling groups are described, for example, in EP 0 580 979, WO 90/05301, WO 90/11511, and WO 92/14138.

Some compounds as used in the immunoassay and method as reported herein are conjugated to a member of a binding pair. The conjugation is, in certain embodiments, performed by chemical binding via N-terminal and/or ε-amino groups (lysine), ε-amino groups of different lysins, carboxy-, sulfhydryl-, hydroxyl- and/or phenolic functional groups of the amino acid backbone of the compound and/or sugar alcohol groups of the carbohydrate structure of the compound. The conjugated compound is, in certain embodiments, a mixture of at least two compounds conjugated to a member of a binding pair, wherein the at least two compounds in the mixture differ in the site at which they are conjugated to the member of the binding pair. For example, the mixture may comprise a conjugation via an amino acid of the amino acid backbone and a conjugation via a sugar alcohol group of a carbohydrate. Also, for example, the mixture may comprise compounds conjugated to the member of a binding pair via different amino acid residues of the amino acid backbone. The expression “different amino acid residue” denotes either two different kinds of amino acids, such as e.g. lysine and aspartic acid, or tyrosine and glutamic acid, or two amino acid residues of the amino acid backbone differing in their position in the amino acid sequence of the compound. In the latter case the amino acid can be of the same kind or of different kind. The expression “differ in the site” denotes a difference either in the kind of site, e.g. amino acid or sugar alcohol group, or in the number of the amino acid of the amino acid backbone, e.g. at which the compound is conjugated to the member of the binding pair.

For direct detection the labeling group can be selected from any known detectable marker groups, such as dyes, luminescent labeling groups such as chemiluminescent groups, e.g. acridinium esters or dioxetanes, or fluorescent dyes, e.g. fluorescein, coumarin, rhodamine, oxazine, resorufin, cyanine and derivatives thereof. Other examples of labeling groups are luminescent metal complexes, such as ruthenium or europium complexes, enzymes, e.g. as used for ELISA or for CEDIA (Cloned Enzyme Donor Immunoassay, e.g. EP-A-0 061 888), and radioisotopes.

Indirect detection systems comprise, for example, that the detection reagent, e.g., the detection antibody is labeled with a first partner of a bioaffine binding pair. Examples of suitable binding pairs are hapten or antigen/antibody, biotin or biotin analogues such as aminobiotin, iminobiotin or desthiobiotin/avidin or streptavidin, sugar/lectin, nucleic acid or nucleic acid analogue/complementary nucleic acid, and receptor/ligand, e.g., steroid hormone receptor/steroid hormone. Preferred first binding pair members comprise hapten, antigen and hormone. Especially preferred are haptens like digoxin and biotin and analogues thereof. The second partner of such binding pair, e.g. an antibody, streptavidin, etc., usually is labeled to allow for direct detection, e.g., by the labels as mentioned above.

Immunoassays are well known to the skilled artisan. Methods for carrying out such assays as well as practical applications and procedures are summarized in related textbooks. Examples of related textbooks are Tijssen, P., Preparation of enzyme-antibody or other enzyme-macromolecule conjugates (in: “Practice and theory of enzyme immunoassays” (1990), pp. 221-278, Eds. R. H. Burdon and v. P. H. Knippenberg, Elsevier, Amsterdam) and various volumes of “Methods in Enzymology” (Eds. S. P. Colowick, N. O. Caplan, Academic Press), dealing with immunological detection methods, especially volumes 70, 73, 74, 84, 92 and 121.

In all the above immunological detection methods reagent conditions are chosen which allow for binding of the reagents employed, e.g. for binding of an antibody to its corresponding antigen. The skilled artisan refers to the result of such binding event by using the term complex. The complex formed in an assay method according to the present invention is correlated by state of the art procedures to the corresponding concentration of said therapeutic antibody. Depending on the detection reagent employed, this correlating step will result in the concentration of total, active or antigen-bound therapeutic antibody.

The methods and immunoassays according to the current invention are in vitro methods and immunoassays.

### III. EXAMPLES

The following are examples of methods and compositions of the invention. It is understood that various other embodiments may be practiced, given the general description provided above.

### Example 1

### Materials and Methods

General information regarding the nucleotide sequences of human immunoglobulins light and heavy chains is given in: Kabat, E. A., et al., Sequences of Proteins of Immunological Interest, 5th ed., Public Health Service, National Institutes of Health, Bethesda, MD (1991). Amino acids of antibody chains are numbered and referred to according to numbering according to Kabat (Kabat, E. A., et al., Sequences of Proteins of Immunological Interest, 5th ed., Public Health Service, National Institutes of Health, Bethesda, MD (1991)).

### Recombinant DNA Techniques

Standard methods can be used to manipulate DNA as described in Sambrook, J. et al., Molecular Cloning: A laboratory manual; Cold Spring Harbor Laboratory Press, Cold Spring Harbor, New York, 1989. The molecular biological reagents are used according to the manufacturer's instructions.

### Gene Synthesis

Desired gene segments can be prepared from oligonucleotides made by chemical synthesis. The long gene segments, which can be flanked by singular restriction endonuclease cleavage sites, can be assembled by annealing and ligating oligonucleotides including PCR amplification and subsequently cloned via the indicated restriction sites. The DNA sequences of the subcloned gene fragments can be confirmed by DNA sequencing.

### DNA Sequence Determination

DNA sequences can be determined by double strand sequencing.

### DNA and Protein Sequence Analysis and Sequence Data Management

The GCG's (Genetics Computer Group, Madison, Wisconsin) software package version 10.2 and Infomax's Vector NTI Advance suite version 8.0 can be used for sequence creation, mapping, analysis, annotation and illustration.

### Expression Vectors

For the expression of antibodies, expression plasmids for transient expression (e.g. in HEK293 cells) based either on a cDNA organization with or without a CMV-intron A promoter or on a genomic organization with a CMV promoter can be applied.

Beside the antibody expression cassette the vector may contain:
an origin of replication which allows replication of this plasmid in E. coli, and
a β-lactamase gene which confers ampicillin resistance in E. coli.

The transcription unit of the antibody gene may be composed of the following elements:
unique restriction site(s) at the 5′ end
the immediate early enhancer and promoter from the human cytomegalovirus,
the intron A sequence in the case of cDNA organization,
a 5′-untranslated region derived from a human antibody gene,
an immunoglobulin heavy chain signal sequence,
the respective antibody chain encoding nucleic acid either as cDNA or with genomic exon-intron organization,
a 3′ untranslated region with a polyadenylation signal sequence, and
unique restriction site(s) at the 3′ end.

The fusion genes encoding the antibody chains can be generated by PCR and/or gene synthesis and assembled by known recombinant methods and techniques by connection of the according nucleic acid segments e.g. using unique restriction sites in the respective vectors. The subcloned nucleic acid sequences can be verified by DNA sequencing. For transient transfections larger quantities of the plasmids can be prepared by plasmid preparation from transformed E. coli cultures.

### Cell Culture Techniques

Standard cell culture techniques as described in Current Protocols in Cell Biology (2000), Bonifacino, J. S., Dasso, M., Harford, J. B., Lippincott-Schwartz, J. and Yamada, K. M. (eds.), John Wiley & Sons, Inc., can be used.

### Recombinant Antibody Production by Transient Transfections in HEK293 System

Antibodies can be produced by transient expression. Therefore a transfection with the respective plasmids using the HEK293 system (Invitrogen) according to the manufacturer's instruction can be done. Briefly, HEK293 cells (Invitrogen) growing in suspension either in a shake flask or in a stirred fermenter in serum-free FreeStyle™ 293 expression medium (Invitrogen) can be transfected with a mix of the respective expression plasmids and 293fectin™ or fectin (Invitrogen). For 2 L shake flask (Corning), HEK293 cells can be seeded at a density of 1.0*10⁶ cells/mL in 600 mL and incubated at 120 rpm, 8% CO₂. On the next day the cells can be transfected at a cell density of approx. 1.5*10⁶ cells/mL with approx. 42 mL of a mixture of A) 20 mL Opti-MEM medium (Invitrogen) comprising 600 μg total plasmid DNA (1 μg/mL) and B) 20 mL Opti-MEM medium supplemented with 1.2 mL 293 fectin or fectin (2 μL/mL). According to the glucose consumption glucose solution can be added during the course of the fermentation. The supernatant containing the secreted antibody is generally harvested after 5-10 days and antibodies can be either directly purified from the supernatant or the supernatant is frozen and stored.

### Protein Determination

The protein concentration of purified antibodies and derivatives can be determined by determining the optical density (OD) at 280 nm, using the molar extinction coefficient calculated based on the amino acid sequence according to Pace, et al., Protein Science 4 (1995) 2411-1423.

### Antibody Concentration Determination in Supernatants

The concentration of antibodies and derivatives in cell culture supernatants can be estimated by immunoprecipitation with protein A agarose-beads (Roche Diagnostics GmbH, Mannheim, Germany). Therefore, 60 μL protein A Agarose beads can be washed three times in TBS-NP40 (50 mM Tris buffer, pH 7.5, supplemented with 150 mM NaCl and 1% Nonidet-P40). Subsequently, 1-15 mL cell culture supernatant can be applied to the protein A Agarose beads pre-equilibrated in TBS-NP40. After incubation for at 1 hour at room temperature the beads can be washed on an Ultrafree-MC-filter column (Amicon) once with 0.5 mL TBS-NP40, twice with 0.5 mL 2× phosphate buffered saline (2×PBS, Roche Diagnostics GmbH, Mannheim, Germany) and briefly four times with 0.5 mL 100 mM Na-citrate buffer (pH 5.0). Bound antibody can be eluted by addition of 35 μL NuPAGE® LDS sample buffer (Invitrogen). Half of the sample can be combined with NuPAGE® sample reducing agent or left unreduced, respectively, and heated for 10 min at 70° C. Consequently, 5-30 μL can be applied to a 4-12% NuPAGE® Bis-Tris SDS-PAGE gel (Invitrogen) (with MOPS buffer for non-reduced SDS-PAGE and MES buffer with NuPAGE® antioxidant running buffer additive (Invitrogen) for reduced SDS-PAGE) and stained with Coomassie Blue.

The concentration of the antibodies in cell culture supernatants can be quantitatively measured by affinity HPLC chromatography. Briefly, cell culture supernatants containing antibodies that bind to protein A can be applied to an Applied Biosystems Poros A/20 column in 200 mM KH₂PO₄, 100 mM sodium citrate, pH 7.4 and eluted with 200 mM NaCl, 100 mM citric acid, pH 2.5 on an Agilent HPLC 1100 system. The eluted antibody can be quantified by UV absorbance and integration of peak areas. A purified standard IgG1 antibody served as a standard.

Alternatively, the concentration of antibodies and derivatives in cell culture supernatants can be measured by Sandwich-IgG-ELISA. Briefly, Strepta Well High Bind Streptavidin A-96 well microtiter plates (Roche Diagnostics GmbH, Mannheim, Germany) can be coated with 100 μL/well biotinylated anti-human IgG capture molecule F(ab)2-anti-human Fcgamma antibody-BI (Dianova) at 0.1 μg/mL for 1 hour at room temperature or alternatively overnight at 4° C. and subsequently washed three times with 200 μL/well PBS, 0.05% Tween (PBST, Sigma). Thereafter, 100 μL/well of a dilution series in PBS (Sigma) of the respective antibody containing cell culture supernatants can be added to the wells and incubated for 1-2 hour on a shaker at room temperature. The wells can be washed three times with 200 μL/well PBST and bound antibody was detected with 100 μL F(ab)2-anti-human Fcgamma antibody-POD (Dianova) at 0.1 μg/mL as the detection antibody by incubation for 1-2 hours on a shaker at room temperature. Unbound detection antibody can be removed by washing three times with 200 μL/well PBST. The bound detection antibody can be detected by addition of 100 μL ABTS/well followed by incubation. Determination of absorbance was performed on a Tecan Fluor Spectrometer at a measurement wavelength of 405 nm (reference wavelength 492 nm).

### Preparative Antibody Purification

Antibodies can be purified from filtered cell culture supernatants referring to standard protocols. In brief, antibodies can be applied to a protein A Sepharose column (GE Healthcare) and washed with PBS. Elution of antibodies can be achieved at pH 2.8 followed by immediate neutralization. Aggregated protein can be separated from monomeric antibodies by size exclusion chromatography (Superdex 200, GE Healthcare) in PBS or in 20 mM Histidine buffer comprising 150 mM NaCl (pH 6.0). Monomeric antibody fractions can be pooled, concentrated (if required) using e.g., a MILLIPORE Amicon Ultra (30 MWCO) centrifugal concentrator, frozen and stored at −20° C. or −80° C. Part of the samples can be provided for subsequent protein analytics and analytical characterization e.g. by SDS-PAGE, size exclusion chromatography (SEC) or mass spectrometry.

### SDS-PAGE

The NuPAGE® Pre-Cast gel system (Invitrogen) can be used according to the manufacturer's instruction. In particular, 10% or 4-12% NuPAGE® Novex® Bis-TRIS Pre-Cast gels (pH 6.4) and a NuPAGE® MES (reduced gels, with NuPAGE® antioxidant running buffer additive) or MOPS (non-reduced gels) running buffer can be used.

### CE-SDS

Purity and antibody integrity can be analyzed by CE-SDS using microfluidic Labchip technology (PerkinElmer, USA). Therefore, 5 μL of antibody solution can be prepared for CE-SDS analysis using the HT Protein Express Reagent Kit according manufacturer's instructions and analyzed on LabChip GXII system using a HT Protein Express Chip. Data can be analyzed using LabChip GX Software.

### Analytical Size Exclusion Chromatography

Size exclusion chromatography (SEC) for the determination of the aggregation and oligomeric state of antibodies can be performed by HPLC chromatography. Briefly, protein A purified antibodies can be applied to a Tosoh TSKgel G3000SW column in 300 mM NaCl, 50 mM KH₂PO₄/K₂HPO₄ buffer (pH 7.5) on an Dionex Ultimate® system (Thermo Fischer Scientific), or to a Superdex 200 column (GE Healthcare) in 2×PBS on a Dionex HPLC-System. The eluted antibody can be quantified by UV absorbance and integration of peak areas. BioRad Gel Filtration Standard 151-1901 served as a standard.

### Mass Spectrometry

The antibodies can be deglycosylated with N-Glycosidase F in a phosphate or Tris buffer at 37° C. for up to 17 h at a protein concentration of 1 mg/mL. The limited LysC (Roche Diagnostics GmbH, Mannheim, Germany) digestions can be performed with 100 μg deglycosylated antibody in a Tris buffer (pH 8) at room temperature for 120 hours, or at 37° C. for 40 min, respectively. Prior to mass spectrometry the samples can be desalted via HPLC on a Sephadex G25 column (GE Healthcare). The total mass was determined via ESI-MS on a maXis 4G UHR-QTOF MS system (Bruker Daltonik) equipped with a TriVersa NanoMate source (Advion).

### Production of Antibodies Using Hybridoma

Hybridoma cell lines are inoculated at initial cell densities (live cells) between 1.0×10⁵ and 2.2×10⁵ cells per mL in RPMI 1640 supplemented with 10% FCS, and commonly used supplements and expanded in a T-flask (Celline, IBS) for a period of approximately three weeks. Purification of the antibodies from the culture supernatants are done according to standard protein chemical methods, e.g. as those reported in Bruck, C., et al., Methods Enzymol. 121 (1986) 587-596.

### Example 2

### Characterization of the Monomers of the Multimer According to the Invention

Biotinylated anti-PG Fc-region antibody or biotinylated anti-AAA Fc-region antibody, respectively, was bound to the wells of a streptavidin-coated multi-well plate (SA-MTP) to produce a capture plate. Excess of unbound antibody was removed by washing. Sample/standard antibodies spiked in human and cynomolgus monkey serum (10% final concentration) was added to wells of the SA-MTP multi-well plate coated with the capture plate and incubated for 1 hour at room temperature. After washing, the wells were incubated with digoxygenylated anti-human kappa antibody M1.7.10 (see e.g. WO 2011/048043, incorporated herein by reference). After washing the bound digoxygenylated anti-human kappa antibody complex was incubated with a horseradish peroxidase (HRP) labelled anti-digoxygenin antibody. After another washing step, an ABTS solution was added to the wells and incubated. The product of the color reaction was measured by Elisa reader at 405 nm wavelength (reference wavelength: 490 nm). Absorbance values of each sample or standard were determined in triplicates.

The following Table shows the extinction values determined for an anti-VEGF/ANG2 antibody with the mutations P329G, L234A, L235A, 1253A, H310A, and H435A in serum with the anti-variant (human) Fc-region antibody M1.3.17 (SEQ ID NO: 03 and 04) as reported herein as capture antibody.

| anti-VEGF/ANG2 | signal |
|---|---|
| antibody [ng/mL] | [OD405 nm] |
| 10 | 2.2255 |
| 5 | 1.6010 |
| 2.5 | 0.9865 |
| 1.25 | 0.5730 |
| 0.625 | 0.3140 |
| 0.3125 | 0.1815 |
| 0.15625 | 0.1075 |
| 0 | 0.0740 |

The following Table shows the extinction values determined for antibodies of different specificity with different mutations in the Fc-region using different antibodies as reported herein as capture and tracer antibodies.
assay B: capture antibody: M1.6.22-Bi/M1.7.24-Bi/M1.3.17-Bi
   tracer antibody: 1.7.10-Dig
assay C: capture antibody: M1.6.22-Bi/M1.7.24-Bi/M1.3.17-Bi
   tracer compound: FcγRI-Dig
M1.6.22=anti-AAA variant Fc-region antibody;
M1.7.10=anti-IgG1 kappa antibody;
M1.7.24=anti-PG variant Fc-region antibody;
M1.3.17=anti-PG variant Fc-region antibody;

### Samples:

1) anti-VEGF/ANG2 antibody (IgG1 subclass with mutations P329G/L234A/L235A/1253A/H310A/H435A);
2) anti-VEGF/ANG2 antibody (IgG1 subclass with mutations P329G/L234A/L235A);
3) anti-IGF-1R antibody (IgG1 subclass with mutations I253A/H310A/H435A);
4) anti-P-Selectin antibody (IgG4 subclass with mutations S228P/L235E);
5) anti-VEGF/ANG2 antibody (wild-type IgG1 subclass).

| | | | | concen- | |
|---|---|---|---|---|---|
| | | | | tration | signal |
| assay | capture | tracer | antibody | [ng/mL] | [OD405 nm] |
| B | M1.6.22-Bi | M1.7.10-Dig | 1 | 100 | 0.052 |
| | | | | 50 | 0.050 |
| | | | | 25 | 0.044 |
| | | | | 13 | 0.041 |
| | | | | 6 | 0.039 |
| | | | | 3 | 0.039 |
| | | | | 2 | 0.041 |
| | | | | 0 | 0.047 |
| B | M1.6.22-Bi | M1.7.10-Dig | 2 | 100 | 0.210 |
| | | | | 50 | 0.127 |
| | | | | 25 | 0.092 |
| | | | | 13 | 0.066 |
| | | | | 6 | 0.053 |
| | | | | 3 | 0.044 |
| | | | | 2 | 0.041 |
| | | | | 0 | 0.038 |
| B | M1.6.22-Bi | M1.7.10-Dig | 3 | 100 | 2.898 |
| | | | | 50 | 2.900 |
| | | | | 25 | 2.894 |
| | | | | 13 | 2.849 |
| | | | | 6 | 2.734 |
| | | | | 3 | 2.259 |
| | | | | 2 | 1.482 |
| | | | | 0 | 0.037 |
| B | M1.6.22-Bi | M1.7.10-Dig | 4 | 100 | 0.051 |
| | | | | 50 | 0.047 |
| | | | | 25 | 0.048 |
| | | | | 13 | 0.042 |
| | | | | 6 | 0.040 |
| | | | | 3 | 0.039 |
| | | | | 2 | 0.032 |
| | | | | 0 | 0.038 |
| B | M1.6.22-Bi | M1.7.10-Dig | 5 | 100 | 0.061 |
| | | | | 50 | 0.049 |
| | | | | 25 | 0.047 |
| | | | | 13 | 0.046 |
| | | | | 6 | 0.039 |
| | | | | 3 | 0.037 |
| | | | | 2 | 0.036 |
| | | | | 0 | 0.037 |
| B | M1.7.24-Bi | M1.7.10-Dig | 1 | 100 | 0.057 |
| | | | | 50 | 0.060 |
| | | | | 25 | 0.047 |
| | | | | 13 | 0.053 |
| | | | | 6 | 0.053 |
| | | | | 3 | 0.040 |
| | | | | 2 | 0.049 |
| | | | | 0 | 0.049 |
| B | M1.7.24-Bi | M1.7.10-Dig | 2 | 100 | 3.235 |
| | | | | 50 | 3.167 |
| | | | | 25 | 3.159 |
| | | | | 13 | 3.148 |
| | | | | 6 | 2.981 |
| | | | | 3 | 2.513 |
| | | | | 2 | 1.730 |
| | | | | 0 | 0.046 |
| B | M1.7.24-Bi | M1.7.10-Dig | 3 | 100 | 0.098 |
| | | | | 50 | 0.064 |
| | | | | 25 | 0.048 |
| | | | | 13 | 0.043 |
| | | | | 6 | 0.039 |
| | | | | 3 | 0.046 |
| | | | | 2 | 0.047 |
| | | | | 0 | 0.040 |
| B | M1.7.24-Bi | M1.7.10-Dig | 4 | 100 | 0.050 |
| | | | | 50 | 0.043 |
| | | | | 25 | 0.040 |
| | | | | 13 | 0.037 |
| | | | | 6 | 0.037 |
| | | | | 3 | 0.031 |
| | | | | 2 | 0.033 |
| | | | | 0 | 0.035 |
| B | M1.7.24-Bi | M1.7.10-Dig | 5 | 100 | 0.047 |
| | | | | 50 | 0.040 |
| | | | | 25 | 0.041 |
| | | | | 13 | 0.040 |
| | | | | 6 | 0.038 |
| | | | | 3 | 0.033 |
| | | | | 2 | 0.034 |
| | | | | 0 | 0.039 |
| B | M1.3.17-Bi | M1.7.10-Dig | 1 | 100 | 0.035 |
| | | | | 50 | 0.035 |
| | | | | 25 | 0.035 |
| | | | | 13 | 0.034 |
| | | | | 6 | 0.035 |
| | | | | 3 | 0.036 |
| | | | | 2 | 0.036 |
| | | | | 0 | 0.035 |
| B | M1.3.17-Bi | M1.7.10-Dig | 2 | 100 | 0.216 |
| | | | | 50 | 0.215 |
| | | | | 25 | 0.213 |
| | | | | 13 | 0.217 |
| | | | | 6 | 0.196 |
| | | | | 3 | 0.165 |
| | | | | 2 | 0.125 |
| | | | | 0 | 0.038 |
| B | M1.3.17-Bi | M1.7.10-Dig | 3 | 100 | 0.050 |
| | | | | 50 | 0.048 |
| | | | | 25 | 0.046 |
| | | | | 13 | 0.044 |
| | | | | 6 | 0.045 |
| | | | | 3 | 0.047 |
| | | | | 2 | 0.047 |
| | | | | 0 | 0.045 |
| B | M1.3.17-Bi | M1.7.10-Dig | 4 | 100 | 0.048 |
| | | | | 50 | 0.049 |
| | | | | 25 | 0.047 |
| | | | | 13 | 0.047 |
| | | | | 6 | 0.048 |
| | | | | 3 | 0.050 |
| | | | | 2 | 0.049 |
| | | | | 0 | 0.049 |
| B | M1.3.17-Bi | M1.7.10-Dig | 5 | 100 | 0.043 |
| | | | | 50 | 0.042 |
| | | | | 25 | 0.043 |
| | | | | 13 | 0.042 |
| | | | | 6 | 0.046 |
| | | | | 3 | 0.044 |
| | | | | 2 | 0.043 |
| | | | | 0 | 0.043 |
| C | M1.6.22-Bi | FcγRI-Dig | 1 | 100 | 0.079 |
| | | | | 50 | 0.077 |
| | | | | 25 | 0.081 |
| | | | | 13 | 0.064 |
| | | | | 6 | 0.060 |
| | | | | 3 | 0.066 |
| | | | | 2 | 0.077 |
| | | | | 0 | 0.077 |
| C | M1.6.22-Bi | FcγRI-Dig | 2 | 100 | 0.076 |
| | | | | 50 | 0.057 |
| | | | | 25 | 0.063 |
| | | | | 13 | 0.061 |
| | | | | 6 | 0.060 |
| | | | | 3 | 0.068 |
| | | | | 2 | 0.072 |
| | | | | 0 | 0.064 |
| C | M1.6.22-Bi | FcγRI-Dig | 3 | 100 | 2.096 |
| | | | | 50 | 1.059 |
| | | | | 25 | 0.401 |
| | | | | 13 | 0.175 |
| | | | | 6 | 0.104 |
| | | | | 3 | 0.083 |
| | | | | 2 | 0.082 |
| | | | | 0 | 0.069 |
| C | M1.6.22-Bi | FcγRI-Dig | 4 | 100 | 0.069 |
| | | | | 50 | 0.054 |
| | | | | 25 | 0.060 |
| | | | | 13 | 0.052 |
| | | | | 6 | 0.051 |
| | | | | 3 | 0.050 |
| | | | | 2 | 0.067 |
| | | | | 0 | 0.077 |
| C | M1.6.22-Bi | FcγRI-Dig | 5 | 100 | 0.076 |
| | | | | 50 | 0.067 |
| | | | | 25 | 0.066 |
| | | | | 13 | 0.070 |
| | | | | 6 | 0.064 |
| | | | | 3 | 0.069 |
| | | | | 2 | 0.077 |
| | | | | 0 | 0.069 |
| C | M1.7.24-Bi | FcγRI-Dig | 1 | 100 | 0.050 |
| | | | | 50 | 0.084 |
| | | | | 25 | 0.082 |
| | | | | 13 | 0.085 |
| | | | | 6 | 0.079 |
| | | | | 3 | 0.094 |
| | | | | 2 | 0.085 |
| | | | | 0 | 0.076 |
| C | M1.7.24-Bi | FcγRI-Dig | 2 | 100 | 0.082 |
| | | | | 50 | 0.094 |
| | | | | 25 | 0.083 |
| | | | | 13 | 0.060 |
| | | | | 6 | 0.060 |
| | | | | 3 | 0.057 |
| | | | | 2 | 0.093 |
| | | | | 0 | 0.092 |
| C | M1.7.24-Bi | FcγRI-Dig | 3 | 100 | 0.110 |
| | | | | 50 | 0.095 |
| | | | | 25 | 0.073 |
| | | | | 13 | 0.058 |
| | | | | 6 | 0.069 |
| | | | | 3 | 0.077 |
| | | | | 2 | 0.074 |
| | | | | 0 | 0.086 |
| C | M1.7.24-Bi | FcγRI-Dig | 4 | 100 | 0.080 |
| | | | | 50 | 0.066 |
| | | | | 25 | 0.070 |
| | | | | 13 | 0.066 |
| | | | | 6 | 0.053 |
| | | | | 3 | 0.053 |
| | | | | 2 | 0.056 |
| | | | | 0 | 0.076 |
| C | M1.7.24-Bi | FcγRI-Dig | 5 | 100 | 0.073 |
| | | | | 50 | 0.066 |
| | | | | 25 | 0.063 |
| | | | | 13 | 0.057 |
| | | | | 6 | 0.057 |
| | | | | 3 | 0.053 |
| | | | | 2 | 0.058 |
| | | | | 0 | 0.073 |
| C | M1.3.17-Bi | FcγRI-Dig | 1 | 100 | 1.194 |
| | | | | 50 | 1.167 |
| | | | | 25 | 1.074 |
| | | | | 13 | 1.137 |
| | | | | 6 | 1.171 |
| | | | | 3 | 1.161 |
| | | | | 2 | 1.171 |
| | | | | 0 | 1.176 |
| C | M1.3.17-Bi | FcγRI-Dig | 2 | 100 | 1.222 |
| | | | | 50 | 1.211 |
| | | | | 25 | 1.214 |
| | | | | 13 | 1.226 |
| | | | | 6 | 1.215 |
| | | | | 3 | 1.222 |
| | | | | 2 | 1.221 |
| | | | | 0 | 1.233 |
| C | M1.3.17-Bi | FcγRI-Dig | 3 | 100 | 1.204 |
| | | | | 50 | 1.214 |
| | | | | 25 | 1.203 |
| | | | | 13 | 1.213 |
| | | | | 6 | 1.212 |
| | | | | 3 | 1.210 |
| | | | | 2 | 1.260 |
| | | | | 0 | 1.217 |
| C | M1.3.17-Bi | FcγRI-Dig | 4 | 100 | 1.170 |
| | | | | 50 | 1.153 |
| | | | | 25 | 1.166 |
| | | | | 13 | 1.161 |
| | | | | 6 | 1.175 |
| | | | | 3 | 1.188 |
| | | | | 2 | 1.191 |
| | | | | 0 | 1.189 |
| C | M1.3.17-Bi | FcγRI-Dig | 5 | 100 | 1.183 |
| | | | | 50 | 1.161 |
| | | | | 25 | 1.163 |
| | | | | 13 | 1.166 |
| | | | | 6 | 1.173 |
| | | | | 3 | 1.187 |
| | | | | 2 | 1.190 |
| | | | | 0 | 1.197 |

assay D: capture antibody: M1.7.24-Bi/M1.3.17-Bi tracer antibody: 1.7.10-Dig/M1.19.31-Dig
M1.7.10=anti-IgG1 kappa antibody
M1.19.31=anti-IgG1 kappa antibody
M1.7.24=anti-PGLALA variant Fc-region antibody
M1.3.17=anti-PGLALA variant Fc-region antibody

### Samples:

6) anti-Dig antibody (IgG1 subclass with mutations P329G/L234A/L235A)

| | | | | signal | |
|---|---|---|---|---|---|
| | | | | 10% | signal |
| | | | signal | cynomolgus | 10% human |
| | | | buffer | serum | serum |
| assay | capture | tracer | [OD405 nm] | [OD405 nm] | [OD405 nm] |
| D | M1.3.17-Bi | M1.7.10-Dig | 0.053 | 2.116 | 2.656 |
| | | | 0.038 | 1.177 | 2.550 |
| | | | 0.037 | 0.485 | 2.508 |
| | | | 0.032 | 0.194 | 2.482 |
| | | | 0.031 | 0.096 | 2.489 |
| | | | 0.032 | 0.065 | 2.529 |
| | | | 0.029 | 0.052 | 2.533 |
| | | | 0.031 | 0.044 | 2.514 |
| D | M1.3.17-Bi | M1.19.31-Dig | 0.050 | 1.874 | 2.583 |
| | | | 0.041 | 0.872 | 2.137 |
| | | | 0.040 | 0.357 | 1.843 |
| | | | 0.037 | 0.162 | 1.728 |
| | | | 0.036 | 0.105 | 1.706 |
| | | | 0.033 | 0.082 | 1.671 |
| | | | 0.033 | 0.075 | 1.706 |
| | | | 0.036 | 0.077 | 1.802 |
| D | M1.7.24-Bi | M1.7.10-Dig | 0.062 | 2.367 | 2.738 |
| | | | 0.046 | 1.413 | 2.609 |
| | | | 0.039 | 0.575 | 2.484 |
| | | | 0.034 | 0.223 | 2.457 |
| | | | 0.031 | 0.094 | 2.407 |
| | | | 0.031 | 0.050 | 2.396 |
| | | | 0.034 | 0.041 | 2.438 |
| | | | 0.033 | 0.031 | 2.452 |
| D | M1.7.24-Bi | M1.19.31-Dig | 0.054 | 2.254 | 2.505 |
| | | | 0.051 | 1.168 | 2.044 |
| | | | 0.045 | 0.438 | 1.605 |
| | | | 0.037 | 0.173 | 1.427 |
| | | | 0.033 | 0.078 | 1.335 |
| | | | 0.033 | 0.049 | 1.362 |
| | | | 0.033 | 0.039 | 1.386 |
| | | | 0.033 | 0.035 | 1.423 |

### Example 3

### Formation of the Multivalent Antibody According to the Invention

### Preparation of Anti-PG Antibody Clone 1.7.24 SATP

The monomeric bivalent anti-PG antibody clone 1.7.24 was dialyzed against 100 mM potassium phosphate buffer, containing 150 mM NaCl, pH 7.8, and adjusted to a protein concentration of about 15 mg/mL. N-succinimidyl-3-acetylthiopropionate (SATP) was dissolved in DMSO and added to the antibody solution in a molar ratio of 1:5 (monomeric antibody:SATP). The pH was adjusted to pH 7.1 and the mixture was incubated for 60 min. at 25° C. The reaction was stopped by adding L-lysine at a final concentration of 10 mM and the surplus of SATP was removed by dialysis against 10 mM potassium phosphate buffer, containing 200 mM NaCl, 1 mM EDTA, pH 6.1.

### Preparation of Anti-PG Antibody Clone 1.7.24 MH

The monomeric bivalent anti-PG antibody clone 1.7.24 was dialyzed against 30 mM potassium phosphate buffer, pH 7.4, and thereafter adjusted to a protein concentration of about 25 mg/mL. Maleimidohexanoyl-N-hydroxysuccinimide ester (MHS) was dissolved in DMSO and added to the antibody solution in a molar ratio of 1:6 (monomeric IgG:MHS). The pH was adjusted to pH 7.1 and the mixture was incubated 60 min at 25° C. The reaction was stopped by adding L-lysine to a final concentration of 10 mM, the pH was adjusted to pH 6.2, and the surplus of MHS was removed by dialysis against 10 mM potassium phosphate buffer, containing 200 mM NaCl, 1 mM EDTA, pH 6.1.

### Chemical Conjugation of Anti-PG Antibody Clone 1.7.24 SATP and Anti-PG Antibody Clone 1.7.24 MH

Anti-PG antibody clone 1.7.24 SATP was deacetylated by incubation with 2% (v/v) 1 M hydroxylamine, pH 7.5, and incubated for 45 min. at 25° C. The deacetylated antibody was mixed with anti-PG antibody clone 1.7.24 MH (molar ratio of deacetylated IgG:IgG-MH=1:3) and diluted with 10 mM potassium phosphate buffer, containing 200 mM NaCl, 1 mM EDTA, pH 6.1, to a final concentration of 1.5 mg/mL deacetylated anti-PG antibody clone 1.7.24 and 4.5 mg/mL anti-PG antibody clone 1.7.24 MH. The pH was adjusted to pH 7.1 and the mixture was incubated at 25° C. The conjugation process was analyzed with an analytical gel filtration column (e.g. TSK 3000). The conjugation was stopped after 45 min. by the addition of cysteine to a final concentration of 1 mM. After a further 30 min. incubation time N-methylmaleimide (NMM) was added to a final concentration of 5 mM and the pH was adjusted to pH 7.5. After 60 min. incubation at 25° C. the conjugate was purified and fractionated by size by S300 gel filtration chromatography to eliminate non-conjugated antibodies. Six pools were obtained. Pool 6 contained only monomeric anti-PG antibody clone 1.7.24.

### Example 4—Comparative Example

### The Bivalent, Monomeric Antibody as Calibration Standard in an Anti-Drug Antibody Assay

A dilution series of the monomeric full-length anti-PG Fc-region antibody clone 1.3.17 was prepared as standards for checking the possibility of generating a standard curve.

Biotinylated anti-VEGF/ANG2 antibody with the mutations P329G, L234A, L235A, I253A, H310A, and H435A and digoxygenylated anti-VEGF/ANG2 antibody with the mutations P329G, L234A, L235A, 1253A, H310A, and H435A were pre-incubated with the standards overnight at room temperature. After pre-incubation, the samples were transferred to a streptavidin-coated multi-well plate and incubated for 1 hour at room temperature. Excess of unbound antibody was removed by washing. After a washing step the bound digoxygenylated complexes comprising biotinylated and digoxygenylated anti-VEGF/ANG2 antibody with the mutations P329G, L234A, L235A, 1253A, H310A, and H435A as well as the monomeric, full-length anti-PG Fc-region antibody M1.3.17 (SEQ ID NO: 03 and 04) were detected with an horseradish peroxidase (HRP) labelled anti-digoxygenin-antibody. After a washing step and upon incubation with the respective substrate the HRP present in the formed complex catalyzes the conversion of ABTS into a colored product. The signal was measured by Elisa reader at 405 nm wavelength (reference wavelength: 490 nm). Absorbance values of each serum sample were determined in triplicates.

The following Table shows the extinction values determined for an anti-VEGF/ANG2 antibody with the mutations P329G, L234A, L235A, I253A, H310A, and H435A in serum as capture antibody (biotinylated) as well as tracer antibody (digoxygenylated) with the monomeric, full-length anti-variant (human) Fc-region antibody M1.3.17 (SEQ ID NO: 03 and 04) as standard.

| anti-PG antibody | signal |
|---|---|
| M1.3.17 [ng/mL] | [OD405 nm] |
| 250 | 0.150 |
| 125 | 0.085 |
| 62.5 | 0.063 |
| 31.25 | 0.054 |
| 15.625 | 0.050 |
| 7.8125 | 0.047 |
| 3.90625 | 0.045 |
| 0 | 0.046 |

At a concentration of 125 ng/mL the signal to noise ratio is 1.85. Regulatory authority requirements for sensitivity in immunogenicity assays are 100 ng/mL. Hence, the monomeric bivalent anti-PG antibody M1.3.17 with its low sensitivity is not suitable as positive control or calibration standard for ADA assay. It can be seen that at a concentration of 125 ng/ml the signal is only 1.85 times the signal of the blank and, thus, the threshold required by authorities for a valid assay is not reached.

### Example 5

### Use of the Multivalent Antibody According to the Invention Obtained by Chemical

### Conjugation as Positive Control and Calibration Standard

### Generic Assay:

All steps were performed at room temperature (RT), and samples and quality controls were analyzed in the presence of 5% HPS (human pooled serum). Samples were adjusted to the respective concentrations by dilution in low cross buffer® containing Drug-BI (biotinylated drug antibody) and Drug-DIG (digoxygenylated drug antibody). The incubation with the respective biotinylated capture and digoxygenylated detection antibodies (=drug (therapeutic) antibody) at the respective concentrations was for 2 hours either directly on the SA-MTP (streptavidin-coated multi titer (well) plate) or in a not-derivatized MTP (pre-incubation plate) at RT (room temperature) and with shaking at 450 rpm. Thereafter, samples (100 μL) were transferred to a SA-coated MTP, if required, and incubated for 1 hour at RT (450 rpm). Then, wells were washed three-times (300 μL washing buffer each). After addition of 100 μL polyclonal anti-DIG-S-Fab-HRP conjugate (50 mU/mL) and 1 hour incubation, the plate was washed again (three-times with 300 μL washing buffer each). Finally, 100 μL ABTS substrate per well was added, and color reaction was photometrically assessed at 405 nm (reference wavelength 490 nm). Samples were measured in duplicates and averaged. Measurements were accepted as valid if the precision of duplicates was ≤ 20% of the coefficient of variation (CV).

### Pool Selection

For pool selection a bridging ELISA with 0.115 μg/mL biotinylated non-targeted antibody IL2 fusion (non-targeted IgG-IL2) and 0.230 μg/mL digoxygenylated non-targeted IgG-IL2 without any matrix was used. The assay was directly performed on streptavidin-coated microtiter plate (SA-MTP) with 2 h incubation.

In the first step, Pool 1 to 5 were tested separately in assay buffer with concentrations from 10,000 ng/ml down to 100 ng/ml in 10-times dilution steps.

| assay | matrix | Pool No. | | | | |
|---|---|---|---|---|---|---|
| concen- | concen- | 1 | 2 | 3 | 4 | 5 |
| tration | tration | signal | signal | signal | signal | signal |
| [ng/mL] | [ng/mL] | [AU] | [AU] | [AU] | [AU] | [AU] |
| 500 | 10000 | 3.2150 | 3.0280 | 3.0370 | 3.1990 | 2.4930 |
| 50 | 1000 | 0.9450 | 2.5780 | 1.4980 | 1.0150 | 0.5480 |
| 5 | 100 | 0.1360 | 0.4490 | 0.2360 | 0.1650 | 0.1000 |
| 0.5 | 10 | 0.0420 | 0.0800 | 0.0500 | 0.0440 | 0.0370 |

The pools were diluted 1 to 20 with biotinylated and digoxygenylated non-targeted IgG-IL2-conjugate.

Pool 2, 3 and 4 were pooled together; all three showed a high binding signal in ADA assay described above. The purified multivalent anti-PG antibody pools derived from clone 1.7.24 was concentrated to about 0.5 mg/mL and stored at −80° C.

### Multimer Versus Monomer Comparison

For comparing monomer versus multimer, the following assay was used. The mixed Pool 2, 3, 4 of the multivalent anti-PG antibody derived from clone 1.7.24 and the monomeric bivalent anti-PG antibody clone 1.7.24 were diluted stepwise 1 to 2 starting from 10,000 ng/mL down to 0.6 ng/mL. These samples then were diluted with biotinylated and digoxygenylated non-targeted IgG-IL2-conjugate by a dilution factor of 20 and incubated for two hours on a pre-incubation plate before transferred to SA-MTP. Followed by a peroxidase-conjugated polyclonal anti-DIG antibody and substrate ABTS. Optical density (OD) is measured at 405 nm (with 490 nm reference wavelength).

| assay | matrix | | |
|---|---|---|---|
| concentration | concentration | monomer | multimer |
| [ng/mL] | [ng/mL] | signal [AU] | signal [AU] |
| 500 | 10000 | 0.2145 | 3.0875 |
| 250 | 5000 | 0.1495 | 3.0350 |
| 125 | 2500 | 0.1075 | 3.0845 |
| 63 | 1250 | 0.0805 | 2.8485 |
| 31 | 625 | 0.0670 | 1.8935 |
| 16 | 313 | 0.0595 | 0.9955 |
| 7.8 | 156 | 0.0550 | 0.5395 |
| 3.9 | 78 | 0.0570 | 0.2865 |
| 2.0 | 39 | 0.0515 | 0.1650 |
| 1.0 | 20 | 0.0465 | 0.1140 |
| 0.5 | 10 | 0.0485 | 0.0825 |
| 0.2 | 4.9 | 0.0480 | 0.0645 |
| 0.1 | 2.4 | 0.0475 | 0.0580 |
| 0.06 | 1.2 | 0.0495 | 0.0560 |
| 0.03 | 0.6 | 0.0485 | 0.0525 |
| 0.00 | 0.0 | 0.0485 | 0.0530 |

### Drug Antibodies of Different Format

For checking the generic applicability of the multivalent anti-PG antibody, i.e. if it can be used as positive control and calibration standard independent of the format of the drug antibody, drug antibodies of three different formats were tested: non-targeted IgG-IL2 (interleukin-2 fused to the C-terminus of a heavy chain of a germline antibody, which is not binding to a target), targeted IgG-IL2 (interleukin-2 fused to the C-terminus of a heavy chain of an antibody specifically binding to a therapeutic target) and a TCB (T-cell bispecific format). The concentration of targeted IgG-IL2-BI and -DIG and also of TCB-BI and -DIG were 0.5 μg/mL each. For non-targeted IgG-IL2 the same concentration as used in the pool selection example above was used (0.115 μg/mL drug-BI and 0.230 μg/mL drug-DIG).

Multivalent anti-PG antibody derived from clone 1.7.24 was diluted from 10 μg/mL down to 0.6 ng/ml in a dilution series 1 to 2 in human pooled serum.

Hence, the diluted multivalent anti-PG antibody derived from clone 1.7.24 was diluted with Drug-BI and Drug-DIG by adding 12.5 μL of sample and 237.5 μL Drug-BI/Drug-DIG solution to a pre-incubation plate. After 2 hour incubation the formed complex was added to a SA-MTP and further processed like described before.

| assay | matrix | non- | | |
|---|---|---|---|---|
| concen- | concen- | targeted | targeted | |
| tration | tration | IgG-IL2 | IgG-IL2 | TCB |
| [ng/mL] | [ng/mL] | signal [AU] | signal [AU] | signal [AU] |
| 500 | 10000 | 2.8105 | 3.0070 | 2.9865 |
| 250 | 5000 | 2.1675 | 2.9945 | 2.9940 |
| 125 | 2500 | 1.4010 | 3.0525 | 3.0530 |
| 63 | 1250 | 0.7705 | 2.6275 | 2.8645 |
| 31 | 625 | 0.4080 | 1.6520 | 2.0910 |
| 16 | 312.5 | 0.2295 | 0.8940 | 1.1900 |
| 7.8 | 156 | 0.1370 | 0.4755 | 0.6545 |
| 3.9 | 78 | 0.0880 | 0.2605 | 0.3500 |
| 2.0 | 39 | 0.0615 | 0.1485 | 0.1945 |
| 1.0 | 20 | 0.0485 | 0.0975 | 0.1200 |
| 0.49 | 10 | 0.0450 | 0.0675 | 0.0835 |
| 0.24 | 4.9 | 0.0450 | 0.0550 | 0.0645 |
| 0.12 | 2.4 | 0.0395 | 0.0440 | 0.0555 |
| 0.06 | 1.2 | 0.0420 | 0.0420 | 0.0490 |
| 0.03 | 0.6 | 0.0425 | 0.0400 | 0.0455 |
| 0.00 | 0.0 | 0.0410 | 0.0395 | 0.0425 |

### Example 6

### Use of the Multivalent Antibody According to the Invention Generated by Recombinant Expression as Tetravalent Fusion Protein as Calibration Standard

In this example, a tetravalent form of the anti-PG antibody clone 1.7.24 was used. This tetravalent form has been obtained by fusion of an addition Fab of the anti-PG antibody clone 1.7.24 to each of the heavy chain C-termini.

Variant Fc-region-BI (biotinylated variant Fc-region fragment) and variant Fc-region-DIG (digoxygenylated variant Fc-region fragment) were used as capture and as tracer molecule.

The results are shown in FIG. 10.

### Example 7

### Use of the Multivalent Antibody According to the Invention Generated by Recombinant Expression as IgM as Calibration Standard

In this example, an IgM form of an anti-PG antibody was used. This IgM form has been recombinantly produced. The IgM was used in the ELISA as unpurified supernatant of the cell culture. The supernatant was tested undiluted and serially diluted 1:5 in buffer.

Variant Fc-region-BI (biotinylated variant Fc-region fragment) and the drug antibody in TCB format (digoxygenylated drug antibody) were used as capture and as tracer molecule.

The results are shown in FIG. 11 and the following Table.

| concentration [mg/mL] | signal [U] |
|---|---|
| 1 | 2.9310 |
| 0.2 | 2.8700 |
| 0.04 | 2.0970 |
| 0.008 | 0.2480 |
| 0.0016 | 0.1560 |
| 0.00032 | 0.1400 |
| 0.000064 | 0.1330 |
| 0.0000128 | 0.1280 |

1. An antibody comprising four or six binding sites that specifically bind to a variant immunoglobulin Fc-region of the human IgG1 subclass comprising one, two, three or four amino acid changes compared to a wild-type Fc-region of the human IgG1 subclass, wherein the variant immunoglobulin Fc-region comprises:
i) a glycine residue at position 329 (numbering according to Kabat EU index), or
ii) an alanine residue at positions 253, 310 and 435 (numbering according to Kabat EU index).

2. The antibody of claim 1, wherein the antibody is
i) an antibody multimer comprising at least two covalently linked bivalent, full length antibodies each comprising two binding sites specifically binding to the variant immunoglobulin Fc-region of the human IgG1, or
ii) an antibody multimer comprising at least two covalently linked (Fab′)2 fragments of a bivalent, full-length antibody each comprising two binding sites specifically binding to the variant immunoglobulin Fc-region of the human IgG1 subclass, or
iii) a tetravalent antibody comprising (1) a first and second polypeptide chain each comprising, from N-terminal to C-terminal, a VH region, a CH1 region, a CH2 region, a CH3 region, the CH1 region, and the VH region, and (2) a second and fourth polypeptide chain each comprising, from N-terminal to C-terminal, a VL region and a CL region.

3. The antibody of claim 1, wherein the variant immunoglobulin Fc-region comprises a glycine residue at position 329 (numbering according to Kabat EU index).

4. The antibody of claim 1, wherein each of the binding sites comprises independently of each other either
(1)
(a) a HVR-H1 comprising the amino acid sequence of SEQ ID NO: 09;
(b) a HVR-H2 comprising the amino acid sequence of SEQ ID NO: 12;
(c) a HVR-H3 comprising the amino acid sequence of SEQ ID NO: 16;
(d) a HVR-L1 comprising the amino acid sequence of SEQ ID NO: 23;
(e) a HVR-L2 comprising the amino acid sequence of SEQ ID NO: 26; and
(f) a HVR-L3 comprising the amino acid sequence of SEQ ID NO: 28; or
(2)
(a) a HVR-H1 comprising the amino acid sequence of SEQ ID NO: 10;
(b) a HVR-H2 comprising the amino acid sequence of SEQ ID NO: 13;
(c) a HVR-H3 comprising the amino acid sequence of SEQ ID NO: 17;
(d) a HVR-L1 comprising the amino acid sequence of SEQ ID NO: 23;
(e) a HVR-L2 comprising the amino acid sequence of SEQ ID NO: 26; and
(f) a HVR-L3 comprising the amino acid sequence of SEQ ID NO: 29; or
(3)
(a) a HVR-H1 comprising the amino acid sequence of SEQ ID NO: 10;
(b) a HVR-H2 comprising the amino acid sequence of SEQ ID NO: 14;
(c) a HVR-H3 comprising the amino acid sequence of SEQ ID NO: 18;
(d) a HVR-L1 comprising the amino acid sequence of SEQ ID NO: 23;
(e) a HVR-L2 comprising the amino acid sequence of SEQ ID NO: 26; and
(f) a HVR-L3 comprising the amino acid sequence of SEQ ID NO: 30; or
(4)
(a) a HVR-H1 comprising the amino acid sequence of SEQ ID NO: 20;
(b) a HVR-H2 comprising the amino acid sequence of SEQ ID NO: 21;
(c) a HVR-H3 comprising the amino acid sequence of SEQ ID NO: 22;
(d) a HVR-L1 comprising the amino acid sequence of SEQ ID NO: 32;
(e) a HVR-L2 comprising the amino acid sequence of SEQ ID NO: 34; and
(f) a HVR-L3 comprising the amino acid sequence of SEQ ID NO: 35.

5. The antibody of claim 3, wherein the variant immunoglobulin Fc-region comprises an alanine residue at positions 234 and 235 (numbering according to Kabat EU index).

6. The antibody of claim 2, wherein the antibody multimer is a dimer, a trimer, a tetramer, a pentamer, a hexamer, a heptamer, an octamer, a nonamer, or a decamer.

7. Use of an antibody according to claim 1 as a positive control or a standard in an in vitro (bridging) immunoassay.

8. (canceled)

9. The use according to claim 7, wherein the use is for the generation of a calibration function for quantitative determination of anti-drug antibodies against a drug antibody, wherein the anti-drug antibodies bind to one or more amino acid residue(s) in the Fc-region of the drug antibody that is altered compared to a wild-type Fc-region.

10. An immunoassay system for the determination of the presence and/or amount of anti-drug antibodies in a (serum containing) sample,
wherein the drug antibody comprises a variant immunoglobulin Fc-region of the human IgG1 subclass comprising (i) a glycine residue at position 329 (according to Kabat EU index) or (ii) an alanine residue at positions 253, 310 and 435 (numbering according to Kabat EU index), and wherein the anti-drug antibodies bind the variant immunoglobulin Fc-region of the drug antibody,
wherein the immunoassay system comprises:
a) a capture antibody, wherein the capture antibody is a drug antibody absorbed on or bound to a solid phase;
b) a tracer antibody, wherein the tracer antibody is the drug antibody conjugated to a detectable label; and
c) the antibody according to claim 1 for use as a positive control or as a calibration standard in an immunoassay.

11. The immunoassay system according to claim 10, wherein the immunoassay is a bridging ELISA.

12. The immunoassay system according to claim 10, wherein the antibody of c) is used as calibration standard for the generation of a calibration curve for quantitative determination of anti-drug antibodies against a drug antibody.

13. A method of producing an antibody multimer according to claim 2, comprising chemically conjugating an antibody specifically binding to a variant immunoglobulin Fc-region of the human IgG1 subclass comprising (i) a glycine residue at position 329 (numbering according to Kabat EU index) or (ii) an alanine residue at positions 253, 310 and 435 (numbering according to Kabat EU index) using N-succinimidyl-3-acetylthiopropionate (SATP) and maleimidohexanoyl-N-hydroxysuccinimide (MHS).

14. The method according to claim 13, wherein the multimer is a multimer of full-length antibodies.

15. The method of claim 13,
wherein the antibody comprises
(1)
(a) a HVR-H1 comprising the amino acid sequence of SEQ ID NO: 09;
(b) a HVR-H2 comprising the amino acid sequence of SEQ ID NO: 12;
(c) a HVR-H3 comprising the amino acid sequence of SEQ ID NO: 16;
(d) a HVR-L1 comprising the amino acid sequence of SEQ ID NO: 23;
(e) a HVR-L2 comprising the amino acid sequence of SEQ ID NO: 26; and
(f) a HVR-L3 comprising the amino acid sequence of SEQ ID NO: 28; or
(2)
(a) a HVR-H1 comprising the amino acid sequence of SEQ ID NO: 10;
(b) a HVR-H2 comprising the amino acid sequence of SEQ ID NO: 13;
(c) a HVR-H3 comprising the amino acid sequence of SEQ ID NO: 17;
(d) a HVR-L1 comprising the amino acid sequence of SEQ ID NO: 23;
(e) a HVR-L2 comprising the amino acid sequence of SEQ ID NO: 26; and
(f) a HVR-L3 comprising the amino acid sequence of SEQ ID NO: 29; or
(3)
(a) a HVR-H1 comprising the amino acid sequence of SEQ ID NO: 10;
(b) a HVR-H2 comprising the amino acid sequence of SEQ ID NO: 14;
(c) a HVR-H3 comprising the amino acid sequence of SEQ ID NO: 18;
(d) a HVR-L1 comprising the amino acid sequence of SEQ ID NO: 23;
(e) a HVR-L2 comprising the amino acid sequence of SEQ ID NO: 26; and
(f) a HVR-L3 comprising the amino acid sequence of SEQ ID NO: 30; or
(4)
(a) a HVR-H1 comprising the amino acid sequence of SEQ ID NO: 20;
(b) a HVR-H2 comprising the amino acid sequence of SEQ ID NO: 21;
(c) a HVR-H3 comprising the amino acid sequence of SEQ ID NO: 22;
(d) a HVR-L1 comprising the amino acid sequence of SEQ ID NO: 32;
(e) a HVR-L2 comprising the amino acid sequence of SEQ ID NO: 34; and
(f) a HVR-L3 comprising the amino acid sequence of SEQ ID NO: 35.

16. An antibody comprising four or six binding sites that specifically bind to a variant immunoglobulin Fc-region of the human IgG1 subclass comprising (i) a glycine residue at position 329 (numbering according to Kabat EU index), or (ii) an alanine residue at positions 253, 310 and 435 (numbering according to Kabat EU index),
wherein each of the binding sites comprises independently of each other either
(1)
(a) a HVR-H1 comprising the amino acid sequence of SEQ ID NO: 09;
(b) a HVR-H2 comprising the amino acid sequence of SEQ ID NO: 12;
(c) a HVR-H3 comprising the amino acid sequence of SEQ ID NO: 16;
(d) a HVR-L1 comprising the amino acid sequence of SEQ ID NO: 23;
(e) a HVR-L2 comprising the amino acid sequence of SEQ ID NO: 26; and
(f) a HVR-L3 comprising the amino acid sequence of SEQ ID NO: 28; or
(2)
(a) a HVR-H1 comprising the amino acid sequence of SEQ ID NO: 10;
(b) a HVR-H2 comprising the amino acid sequence of SEQ ID NO: 13;
(c) a HVR-H3 comprising the amino acid sequence of SEQ ID NO: 17;
(d) a HVR-L1 comprising the amino acid sequence of SEQ ID NO: 23;
(e) a HVR-L2 comprising the amino acid sequence of SEQ ID NO: 26; and
(f) a HVR-L3 comprising the amino acid sequence of SEQ ID NO: 29; or
(3)
(a) a HVR-H1 comprising the amino acid sequence of SEQ ID NO: 10;
(b) a HVR-H2 comprising the amino acid sequence of SEQ ID NO: 14;
(c) a HVR-H3 comprising the amino acid sequence of SEQ ID NO: 18;
(d) a HVR-L1 comprising the amino acid sequence of SEQ ID NO: 23;
(e) a HVR-L2 comprising the amino acid sequence of SEQ ID NO: 26; and
(f) a HVR-L3 comprising the amino acid sequence of SEQ ID NO: 30; or
(4)
(a) a HVR-H1 comprising the amino acid sequence of SEQ ID NO: 20;
(b) a HVR-H2 comprising the amino acid sequence of SEQ ID NO: 21;
(c) a HVR-H3 comprising the amino acid sequence of SEQ ID NO: 22;
(d) a HVR-L1 comprising the amino acid sequence of SEQ ID NO: 32;
(e) a HVR-L2 comprising the amino acid sequence of SEQ ID NO: 34; and
(f) a HVR-L3 comprising the amino acid sequence of SEQ ID NO: 35.

17. The antibody of claim 16, wherein each of the binding sites comprise:
(1)
(a) a HVR-H1 comprising the amino acid sequence of SEQ ID NO: 09;
(b) a HVR-H2 comprising the amino acid sequence of SEQ ID NO: 12;
(c) a HVR-H3 comprising the amino acid sequence of SEQ ID NO: 16;
(d) a HVR-L1 comprising the amino acid sequence of SEQ ID NO: 23;
(e) a HVR-L2 comprising the amino acid sequence of SEQ ID NO: 26; and
(f) a HVR-L3 comprising the amino acid sequence of SEQ ID NO: 28; or
(2)
(a) a HVR-H1 comprising the amino acid sequence of SEQ ID NO: 10;
(b) a HVR-H2 comprising the amino acid sequence of SEQ ID NO: 13;
(c) a HVR-H3 comprising the amino acid sequence of SEQ ID NO: 17;
(d) a HVR-L1 comprising the amino acid sequence of SEQ ID NO: 23;
(e) a HVR-L2 comprising the amino acid sequence of SEQ ID NO: 26; and
(f) a HVR-L3 comprising the amino acid sequence of SEQ ID NO: 29; or
(3)
(a) a HVR-H1 comprising the amino acid sequence of SEQ ID NO: 10;
(b) a HVR-H2 comprising the amino acid sequence of SEQ ID NO: 14;
(c) a HVR-H3 comprising the amino acid sequence of SEQ ID NO: 18;
(d) a HVR-L1 comprising the amino acid sequence of SEQ ID NO: 23;
(e) a HVR-L2 comprising the amino acid sequence of SEQ ID NO: 26; and
(f) a HVR-L3 comprising the amino acid sequence of SEQ ID NO: 30; or
(4)
(a) a HVR-H1 comprising the amino acid sequence of SEQ ID NO: 20;
(b) a HVR-H2 comprising the amino acid sequence of SEQ ID NO: 21;
(c) a HVR-H3 comprising the amino acid sequence of SEQ ID NO: 22;
(d) a HVR-L1 comprising the amino acid sequence of SEQ ID NO: 32;
(e) a HVR-L2 comprising the amino acid sequence of SEQ ID NO: 34; and
(f) a HVR-L3 comprising the amino acid sequence of SEQ ID NO: 35.

18. The antibody of claim 16, wherein the antibody is:
i) an antibody multimer comprising at least two covalently linked bivalent, full length antibodies each comprising two binding sites specifically binding to the variant immunoglobulin Fc-region of the human IgG1 subclass, or
ii) an antibody multimer comprising at least two covalently linked (Fab′)2 fragments of a bivalent, full-length antibody each comprising two binding sites specifically binding to the variant immunoglobulin Fc-region of the human IgG1 subclass, or
iii) a tetravalent antibody comprising (1) a first and second polypeptide chain each comprising, from N-terminal to C-terminal, a VH region, a CH1 region, a CH2 region, a CH3 region, the CH1 region, and the VH region, and (2) a second and fourth polypeptide chain each comprising, from N-terminal to C-terminal, a VL region and a CL region.

19. A method of conducting a sandwich or bridging immunoassay for the determination of anti-drug antibodies against a therapeutic antibody comprising a variant immunoglobulin Fc-region, wherein the variant immunoglobulin Fc-region comprises a glycine residue at position 329 (numbering according to Kabat EU index), the method comprising:
a) incubating the antibody of claim 1 with the therapeutic antibody or an Fc-region (fragment) thereof that has been immobilized on a solid surface to form an dimeric complex,
b) incubating said dimeric complex with the therapeutic antibody conjugated to a detectable label to form a ternary complex; and
c) determining the correct/proper function of the sandwich or bridging immunoassay based on detection of the ternary complex formed in (b).

20. The method of claim 19, comprising:
d) separately incubating the therapeutic antibody or an Fc-region (fragment) thereof that has been immobilized on a solid surface with a sample comprising mammalian blood serum, thereby forming a solid-phase-bound drug antibody-anti-drug antibody complex,
e) incubating the solid surface to which the drug antibody-anti-drug antibody complex formed in step a) is bound with the drug antibody or an Fc-region fragment thereof conjugated to a detectable label, and
f) determining the formation of a solid-phase-bound complex in step e) by determining the presence of the detectable label and thereby determining the presence of an anti-drug antibody against the therapeutic antibody.

21. A method of calibrating a sandwich/bridging immunoassay for the determination of anti-drug antibodies against a therapeutic antibody comprising a variant immunoglobulin Fc-region, wherein the variant immunoglobulin Fc-region comprises a glycine residue at position 329 (numbering according to Kabat EU index), the method comprising:
a) separately incubating the therapeutic antibody or an Fc-region (fragment) thereof that has been immobilized on a solid surface with at least two different concentrations of the antibody of claim 1, thereby forming dimeric complexes,
b) incubating each of the dimeric complexes separately with the drug (therapeutic) antibody conjugated to a detectable label to form ternary complexes,
c) determining the amount of each of the ternary complexes formed in step c) by determining the amount of the detectable label, and
d) calculating a calibration curve based on the amounts determined in step c) and thereby calibrating the sandwich/bridging immunoassay.

22. The method of claim 21, comprising:
e) separately incubating the therapeutic antibody or an Fc-region (fragment) thereof that has been immobilized on a solid surface with a sample comprising mammalian blood serum, thereby forming a solid-phase-bound drug antibody-anti-drug antibody complex,
f) incubating the solid surface to which the drug antibody-anti-drug antibody complex formed in step a) is bound with the drug antibody or an Fc-region fragment thereof conjugated to a detectable label, and
g) determining the formation of a solid-phase-bound complex in step e) by determining the presence of the detectable label and thereby determining the presence of an anti-drug antibody against the therapeutic antibody.

23. A kit, comprising:
a) an antibody comprising four or six binding sites that specifically bind to a variant immunoglobulin Fc-region of the human IgG1 subclass comprising one, two, three or four amino acid changes compared to a wild-type Fc-region of the human IgG1 subclass, wherein the variant immunoglobulin Fc-region comprises (i) a glycine residue at position 329 (numbering according to Kabat EU index), or (ii) an alanine residue at positions 253, 310 and 435 (numbering according to Kabat EU index); and
b) instructions for performing the method of claim 19.

24. A kit, comprising:
a) an antibody comprising four or six binding sites that specifically bind to a variant immunoglobulin Fc-region of the human IgG1 subclass comprising one, two, three or four amino acid changes compared to a wild-type Fc-region of the human IgG1 subclass, wherein the variant immunoglobulin Fc-region comprises (i) a glycine residue at position 329 (numbering according to Kabat EU index), or (ii) an alanine residue at positions 253, 310 and 435 (numbering according to Kabat EU index); and
b) instructions for performing the method of claim 21.


SEQUENCE LISTING (37 sequences)

SEQ ID NO: 1 - protein, 137 aa, Mus musculus
MDFGLIFFIVALLKGVLCEVRLLESGGGLVQPGGSLKLSCAASGFDFSRYWMSWVRQAPGKGLEWIGEINPDSRPINSSPSLKDKFIISRDNVKNTLYLQMNELGSEDTALYYCARPLDYGAWFANWGQGTLVSVSA

SEQ ID NO: 2 - protein, 128 aa, Mus musculus
MAWTSLFLSLLALSSGAISQAVVTQESALTTSPGETVTLTCRSSTGAVTTSNYANWVQEKPDHVFSGLIGGTNKRAPGFPARFSGSLIGDKAALTITGAQTEDEAIYFCVLWYSNHWVFGGGTKLTVL

SEQ ID NO: 3 - protein, 137 aa, Mus musculus
MDFGLIFFIVALLKGVQCEVKLLESGGGLVQPGGSLKLSCAASGIDFRRYWMNWVRQAPGKGLEWIGEIDPDSSTINYTPSLKDKFIISRDNAKNTMYLQMRKVRSEDTALYYCARPYDYGAWFAYWGQGTLVTVSA

SEQ ID NO: 4 - protein, 128 aa, Mus musculus
MAWISLLFSLLALSSGAISQAVVTQESALTTSPGETVTLTCRSTTGAVTTSNYANWVQEKPDHLFTGLIGGTNKRAPGVPARFSGSLIGDKAALTITGAQTEDEAIYFCALWYSDHWVFGGGTKLTVL

SEQ ID NO: 5 - protein, 140 aa, Mus musculus
MNFGLSLVFLALILKGVQCEVQLVESGGDLVKPGGSLKLSCAASGFTFSSYGMSWVRQTPDKRLEWVATISSGGSYIYYPDSVKGRFTISRDNAKNTLYLQMSSLKSEDTAMYYCARLGMITTGYAMDYWGQGTSVTVSS

SEQ ID NO: 6 - protein, 112 aa, Mus musculus
DVLMTQTPLSLPVSLGDQASISCRSSQTIVHSTGHTYLEWFLQKPGQSPKLLIYKVSNRFSGVPDRFSGSGSGTDFTLKISRVEAEDLGVYYCFQGSHVPYTFGGGTKLEIK

SEQ ID NO: 7 - protein, 137 aa, Mus musculus
MDFGLIFFIVALLKGVQCEVKLLESGGGLVQPGGSLKLSCAASGFDFSRYWMNWVRQAPGKGLEWIGEITPDSSTINYTPSLKDKFIISRDNAKNTLYLQMIKVRSEDTALYYCVRPYDYGAWFASWGQGTLVTVSA

SEQ ID NO: 8 - protein, 109 aa, Mus musculus
QAVVTQESALTTSPGETVTLTCRSSTGAVTTSNYANWVQEKPDHLFTGLIGGTNKRAPGVPARFSGSLIGDKAALTITGAQTEDEAIYFCALWYSNHWVFGGGTKLTVL

SEQ ID NO: 9 - protein, 5 aa, Mus musculus
RYWMS

SEQ ID NO: 10 - protein, 5 aa, Mus musculus
RYWMN

SEQ ID NO: 11 - protein, 5 aa, synthetic construct
  REGION 1..5: note = HVR-H1 consensus sequence
  VARIANT 5: note = MISC_FEATURE - X denotes a neutral hydrophilic amino acid residue
RYWMX

SEQ ID NO: 12 - protein, 17 aa, Mus musculus
EINPDSRPINSSPSLKD

SEQ ID NO: 13 - protein, 17 aa, Mus musculus
EIDPDSSTINYTPSLKD

SEQ ID NO: 14 - protein, 17 aa, Mus musculus
EITPDSSTINYTPSLKD

SEQ ID NO: 15 - protein, 17 aa, synthetic construct
  REGION 1..17: note = HVR-H2 consensus sequence
  VARIANT 3: note = MISC_FEATURE - X = neutral hydrophilic or acidic amino acid residue
  VARIANT 7: note = MISC_FEATURE - X = neutral hydrophilic or basic amino aicd residue
  VARIANT 8: note = MISC_FEATURE - X = neutral hydrophilic or chain influencing amino aicd residue
  VARIANT 11: note = MISC_FEATURE - X = neutral hydrophilic or aromatic amino aicd residue
  VARIANT 12: note = MISC_FEATURE - X = neutral hydrophilic amino aicd residue
EIXPDSXXINXXPSLKD

SEQ ID NO: 16 - protein, 10 aa, Mus musculus
PLDYGAWFAN

SEQ ID NO: 17 - protein, 10 aa, Mus musculus
PYDYGAWFAY

SEQ ID NO: 18 - protein, 10 aa, Mus musculus
PYDYGAWFAS

SEQ ID NO: 19 - protein, 10 aa, synthetic construct
  REGION 1..10: note = HVR-H3 consensus sequence
  VARIANT 2: note = MISC_FEATURE - X=hydrophobic or aromatic amino acid residue
  VARIANT 10: note = MISC_FEATURE - X=neutral hydrophilic or aromatic amino acid residue
PXDYGAWFAX

SEQ ID NO: 20 - protein, 5 aa, Mus musculus
SYGMS

SEQ ID NO: 21 - protein, 17 aa, Mus musculus
TISSGGSYIYYPDSVKG

SEQ ID NO: 22 - protein, 12 aa, Mus musculus
LGMITTGYAMDY

SEQ ID NO: 23 - protein, 14 aa, Mus musculus
RSSTGAVTTSNYAN

SEQ ID NO: 24 - protein, 14 aa, Mus musculus
RSTTGAVTTSNYAN

SEQ ID NO: 25 - protein, 14 aa, synthetic construct
  REGION 1..14: note = HVR-L1 consensus sequence
  VARIANT 3: note = MISC_FEATURE - X=neutral hydrophilic amino acid residue
  VARIANT 14: note = MISC_FEATURE - X=neutral hydrophilic or acidic amino acid residue
RSXTGAVTTSNYAX

SEQ ID NO: 26 - protein, 7 aa, Mus musculus
GTNKRAP

SEQ ID NO: 27 - protein, 7 aa, synthetic construct
  REGION 1..7: note = HVR-L2 consesus sequence
  VARIANT 4: note = MISC_FEATURE - X=acidic or basic amino acid residue
GTNXRAP

SEQ ID NO: 28 - protein, 9 aa, Mus musculus
VLWYSNHWV

SEQ ID NO: 29 - protein, 9 aa, Mus musculus
ALWYSDHWV

SEQ ID NO: 30 - protein, 9 aa, Mus musculus
ALWYSNHWV

SEQ ID NO: 31 - protein, 9 aa, synthetic construct
  REGION 1..9: note = HVR-L3 consensus sequence
  VARIANT 1: note = misc_feature - Xaa can be any naturally occurring amino acid
  VARIANT 6: note = misc_feature - Xaa can be any naturally occurring amino acid
XLWYSXHWV

SEQ ID NO: 32 - protein, 16 aa, Mus musculus
RSSQTIVHSTGHTYLE

SEQ ID NO: 33 - protein, 16 aa, synthetic construct
  REGION 1..16: note = HVR-L1 consensus sequence
  VARIANT 9: note = MISC_FEATURE - X=neutral or chain orientation influencing amino acid residue
RSSQTIVHXTGHTYLE

SEQ ID NO: 34 - protein, 7 aa, Mus musculus
KVSNRFS

SEQ ID NO: 35 - protein, 9 aa, Mus musculus
FQGSHVPYT

SEQ ID NO: 36 - protein, 121 aa, synthetic construct
EVQLVESGGDLVKPGGSLKLSCAASGFTFSSYGMSWVRQTPDKRLEWVATISSGGSYIYYPDSVKGRFTISRDNAKNTLYLQMSSLKSEDTAMYYCARLGMITTGYAMDYWGQGTSVTVSS

SEQ ID NO: 37 - protein, 119 aa, synthetic construct
EVKLLESGGGLVQPGGSLKLSCAASGFDFSRYWMNWVRQAPGKGLEWIGEITPDSSTINYTPSLKDKFIISRDNAKNTLYLQMIKVRSEDTALYYCVRPYDYGAWFASWGQGTLVTVSA